(19) United States
(12) Patent Application Publication
(10) Pub. No.: US 2012/0059014 A1
(43) Pub. Date: Mar. 8, 2012

(54) Compounds for the Treatment of Metabolic Disorders

(75) Inventors: Oscar Barba, Oxford (GB); Tom Banksia Dupree, Oxford (GB); Peter Timothy Fry, Oxford (GB); Matthew Colin Thor Fyfe, Oxford (GB); Revathy Perpetua Jeevaratnam, Oxford (GB); Thomas Martin Krulle, Oxford (GB); Karen Lesley Schofield, Oxford (GB); Donald Smyth, Oxford (GB); Thomas Staroske, Oxford (GB); Alan John William Stewart, Oxford (GB); David French Stonehouse, Oxford (GB); Simon Andrew Swain, Oxford (GB); David Matthew Withall, Oxford (GB)

(73) Assignee: PROSIDION LIMITED, Oxford (GB)

(21) Appl. No.: 13/255,531

(22) PCT Filed: Mar. 12, 2010
(86) PCT No.: PCT/GB10/50441

§ 371 Date: Nov. 28, 2011

(30) Foreign Application Priority Data
Mar. 12, 2009 (GB) 0904285.4

(51) Int. Cl. A61K 31/506; C07D 413/14; A61K 31/4545; A61K 31/501; A61P 9/12; A61P 3/04; A61P 3/00; A61P 3/08; A61P 3/06; A61P 7/00; C07D 401/14; A61P 3/10

(52) U.S. Cl. 514/252.03; 544/296; 514/273; 546/187; 514/316; 544/319; 514/269; 544/321; 544/238

(57) ABSTRACT

The present invention is directed to therapeutic compounds of the following formula (I)

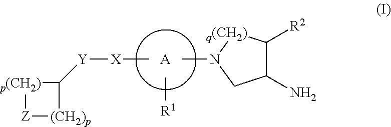

which have activity as agonists of GPR119 and are useful for the treatment of metabolic disorders including type II diabetes.

### BACKGROUND OF THE INVENTION

The present invention is directed to therapeutic compounds useful for the treatment of metabolic disorders including type II diabetes. In particular, the present invention is directed to compounds which have activity as agonists of GPR119.

Drugs aimed at the pathophysiology associated with non-insulin dependent type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) places people at high risk of coronary artery disease, and is characterized by a cluster of risk factors including central obesity (excessive fat tissue in the abdominal region), glucose intolerance, high triglycerides and low HDL cholesterol, and high blood pressure. Myocardial ischemia and microvascular disease is an established morbidity associated with untreated or poorly controlled metabolic syndrome.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

There is a continuing need for novel antidiabetic agents, particularly ones that are well tolerated with few adverse effects and in particular for agents which are weight neutral or preferably cause weight loss.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of diabetes.

GPR119 agonists have been shown to stimulate the release of GLP-1 from the GI tract. In doing so, GPR119 agonists (1) enhance glucose-dependent insulin release from the pancreas leading to improvements in oral glucose tolerance; (2) attenuate disease progression by increasing 3-cell cAMP concentrations; and (3) induce weight loss possibly through GLP-1's ability to reduce food intake.

International Patent Applications WO2005/061489, WO2006/070208, WO2006/067532, WO2006/067531, WO2007/003960, WO2007/003961, WO2007/003962, WO2007/003964, WO2007/116229, WO2007/116230, WO2007/138362, WO2008/081204, WO2008/081205, WO2008/081206, WO2008/081207, WO2008/081208, WO2009/050522, WO2009/050971, WO2010/004343, WO2010/004344, WO2010/004345, WO2010/004347 and WO2010/00166 disclose GPR119 receptor agonists.

Dipeptidyl peptidase IV (DPP-IV) is a ubiquitous, yet highly specific, serine protease that cleaves N-terminal dipeptides from polypeptides with L-proline or L-alanine at the penultimate position. Studies with DPP-IV inhibitors show the principle role of DPP-IV is in the inactivation GLP-1. By extending the duration of action of GLP-1, insulin secretion is stimulated, glucagon release inhibited, and gastric emptying slowed. DPP-IV inhibitors are of use for the treatment of type II diabetes, examples of DPP-IV inhibitors include vildagliptin, sitagliptin, alogliptin and saxagliptin.

The possibility of using a combination of a GPR119 agonist and a DPP-IV inhibitor has been suggested, however this requires the administration of two separately formulated products to the patient or the co-formulation of two active ingredients with the inherent problems of achieving compatibility in the physicochemical, pharmacokinetic and pharmacodynamic properties of the two active ingredients. International Patent Application WO2009/034388, published after the priority date of the present application, discloses compounds having dual activity as agonists of GPR119 and inhibitors of DPP-IV.

The compounds of the invention may also have dual activity as agonists of GPR119 and inhibitors of DPP-IV.

### SUMMARY OF THE INVENTION

The present invention is directed to compounds which have activity as agonists of GPR119 and may also be inhibitors of DPP-IV and are useful for the treatment of metabolic disorders including type II diabetes.

### DETAILED DESCRIPTION OF THE INVENTION

The present invention provides compounds of formula (I) and pharmaceutically acceptable salts thereof:

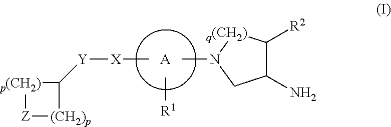

p is 1 or 2;

Z is N—C(O)OR⁴, N—C(O)NR⁴R⁵ or N-heteroaryl which may optionally be substituted by one or two groups selected from C₁₋₄ alkyl, C₃₋₆ cycloalkyl optionally substituted by C₁₋₄ alkyl, C₁₋₄ alkoxy, C₁₋₄haloalkyl and halogen;

X is selected from CR⁶R⁶⁶, O and NR⁷;

Y is a C₂₋₄ alkylene chain optionally substituted by fluoro or methyl, and when X is CR⁶R⁶⁶ one of the carbons in the alkylene chain may be replaced by O;

A is phenyl or a 6-membered heteroaromatic ring containing one or two nitrogen atoms;

R¹ is hydrogen, halo, cyano, C₁₋₄alkyl or C₁₋₄haloalkyl;

q is 1 or 2;

R² is

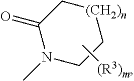

phenyl optionally substituted by one or more halo groups, or pyridyl optionally substituted by one or more halo or methyl groups;

R³ is independently halo or methyl;

n is 0 or 1;

m is 0, 1 or 2;

R⁴ is C₂₋₆ alkyl or C₃₋₆cycloalkyl wherein the cycloalkyl is optionally substituted by C₁₋₄alkyl;

R⁵ is hydrogen or C₁₋₄alkyl;

R⁶ and R⁶⁶ are independently hydrogen, fluoro or C₁₋₄alkyl; and

R⁷ is hydrogen or C₁₋₄alkyl.

In a preferred embodiment the compounds of the invention have the stereochemistry as defined in formula (Ia), such compounds demonstrate DPP-IV inhibitory activity:

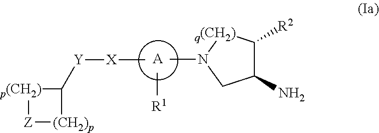

In one of embodiment of the invention each p is independently 1 or 2, i.e. forming a 4-, 5- or 6-membered ring. In another embodiment of the invention each p is the same, i.e. forming a 4- or 6-membered ring. In the compounds of the invention p is preferably 2.

In one embodiment of the invention Z is N—C(O)OR⁴.

R⁴ is preferably C₂₋₆ alkyl.

In a further embodiment of the invention Z is N-heteroaryl which may optionally be substituted by one or two groups selected from C₁₋₄ alkyl, C₃₋₆ cycloalkyl optionally substituted by C₁₋₄alkyl, C₁₋₄ alkoxy, C₁₋₄ haloalkyl and halogen.

When Z is N-heteroaryl preferred heteroaryl groups include oxadiazole and pyrimidine.

X is preferably CR⁶R⁶⁶ or O, more preferably O.

Y is preferably a C₂₋₄ alkylene chain, e.g. a C₃₋₄ alkylene chain, optionally substituted by methyl.

When X is CR⁶R⁶⁶ and one of the carbons in the alkylene chain is replaced by O, then R² is preferably phenyl optionally substituted by one or more halo groups.

A is preferably a meta- or para-linked phenyl or a meta or para linked 6-membered heteroaromatic ring containing one or two nitrogen atoms, more preferably a para-linked phenyl or a para linked 6-membered heteroaromatic ring containing one or two nitrogen atoms.

A is preferably pyridine, pyrimidine, pyrazine or pyridazine, more preferably pyridine or pyrimidine, e.g. 2- or 3-pyridyl or 2- or 5-pyrimidinyl, where the 2-, 3- or 5- refers to the point of attachment of the pyrrolidine or piperidine ring.

R² is preferably phenyl or pyridyl, more preferably phenyl, and even more preferably substituted phenyl.

When R² is phenyl substituted by one or more halo groups it is preferably substituted by 1 to 3 halo groups, the halo groups are preferably fluoro.

When R² is pyridyl it is preferably 2-pyridyl.

When R² is substituted pyridyl it is preferably substituted by 1 to 3 halo or methyl groups, more preferably 1 or 2 methyl groups.

A group of compounds which may be mentioned are those of formula (Ib) and pharmaceutically acceptable salts thereof:

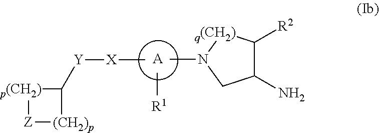

p is 1 or 2;

Z is N—C(O)OR⁴, N—C(O)NR⁴R⁵ or N-heteroaryl which may optionally be substituted by one or two groups selected from C₁₋₄ alkyl, C₁₋₄ alkoxy, C₁₋₄ haloalkyl and halogen;

X is selected from CR⁶R⁶⁶, O and NR⁷;

Y is a C₃₋₄ alkylene chain optionally substituted by fluoro or methyl, and when X is CR⁶R⁶⁶ one of the carbons in the alkylene chain may be replaced by O;

A is phenyl or a 6-membered heteroaromatic ring containing one or two nitrogen atoms;

R¹ is hydrogen, halo, cyano, C₁₋₄ alkyl or C₁₋₄ haloalkyl;

q is 1 or 2;

R² is

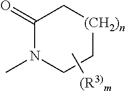

or phenyl optionally substituted by one or more halo groups;

R³ is independently halo or methyl;

n is 0 or 1;

m is 0, 1 or 2;

R⁴ is C₂₋₆ alkyl;

R⁵ is C₁₋₄ alkyl;

R⁶ and R⁶⁶ are independently hydrogen, fluoro or C₁₋₄alkyl; and

R⁷ is hydrogen or C₁₋₄alkyl.

In a preferred embodiment of the compounds formula (Ib) they have the stereochemistry as defined in formula (Ia).

While the preferred groups for each variable have generally been listed above separately for each variable, preferred compounds of this invention include those in which several or each variable in formula (I) is selected from the preferred groups for each variable. Therefore, this invention is intended to include all combinations of preferred listed groups.

Representative compounds of the invention which may be mentioned are those provided in the Examples as the free base or a pharmaceutically acceptable salt thereof.

The molecular weight of the compounds of the invention is preferably less than 800, more preferably less than 600.

As used herein, unless stated otherwise, “alkyl” means carbon chains which may be linear or branched. Examples of alkyl groups include ethyl, propyl, isopropyl, butyl, sec- and tert-butyl.

The term “heteroaryl” rings means 5- or 6-membered N-containing heteroaryl rings containing up to 2 additional heteroatoms selected from N, O and S. Examples of such heteroaryl rings are pyrrolyl, pyrazolyl, imidazolyl, oxazolyl, isoxazolyl, thiazolyl, isothiazolyl, triazolyl, oxadiazolyl, thiadiazolyl, pyridinyl, pyridazinyl, pyrimidinyl, pyrazinyl and triazinyl.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The present invention includes all stereoisomers of the compounds of the invention and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of the invention exists, the present invention includes any possible tautomers and pharmaceutically acceptable salts thereof, and mixtures thereof, except where specifically drawn or stated otherwise.

When the compound of the invention and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of the invention are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, wherein R¹, R², R³, R⁴, R⁵, R⁶, R⁶⁶,R⁷, A, X, Y, Z, m, n, p, q are as defined for formula (I). PG is a protecting group, Hal is halogen

Compounds of formula (I) can be prepared as outlined in Scheme 1. Compounds of formula (IV) can be prepared by SN_{Ar} displacement of suitable haloaromatic compounds of formula (II) with amines of formula (III) under standard conditions, for example, DBU and DMSO at 120° C. Alternatively, compounds of formula (IV) can be prepared by reaction of suitable haloaromatic compounds of formula (II) with amines of formula (III) under Buchwald-Hartwig conditions, such as, Pd₂(dba)₃ and BINAP in a suitable solvent, such as toluene at 110° C. Deprotection of the amine functionality, using standard conditions well known to those with skill in the art, affords compounds of formula (I) as described above.

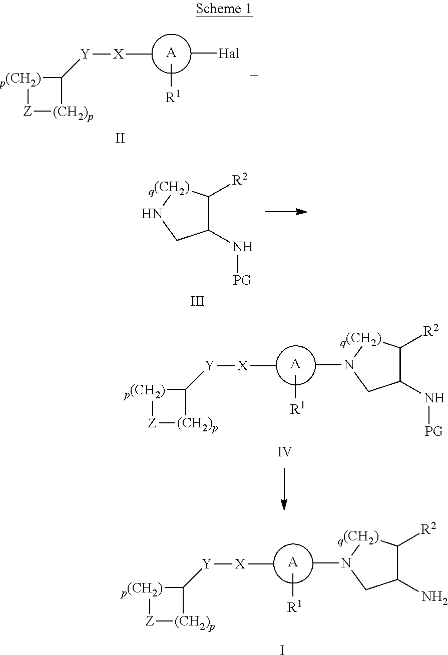

Building blocks of formula (II) where X is O can be prepared as outlined in Scheme 2. Alcohols of formula (V) can be treated with hydroxyaryls of formula (VI) under standard Mitsonobu conditions, for example, using azodicarboxylic dipiperidide and tributylphosphine in a suitable solvent such as toluene.

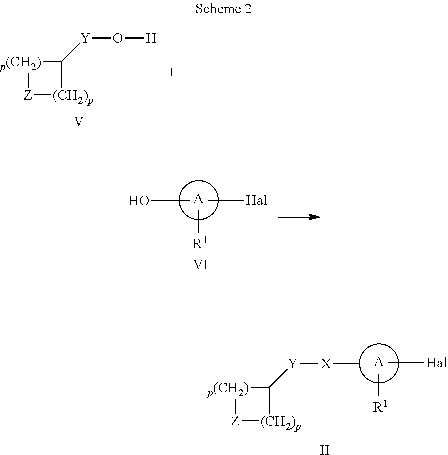

Alternatively, building blocks of formula (II) where X is O can be prepared as outlined in Scheme 3. Mesylates of formula (VII) can be prepared from alcohols of formula (V) under standard conditions, such as, methanesulfonyl chloride and triethylamine in DCM. Compounds of formula (II) can be prepared from mesylates of formula (VII) and hydroxyaryls of formula (VI) under standard conditions, such as K₂CO₃ in DMF at 80° C.

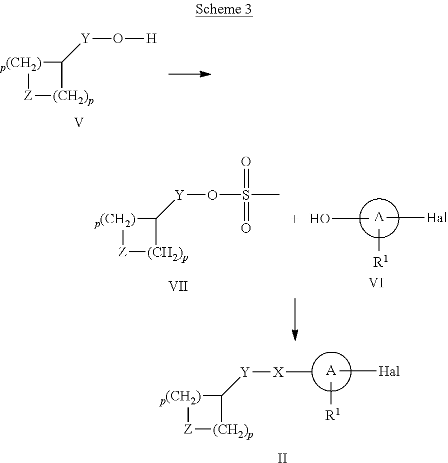

Alternatively, building blocks of formula (II) where X is O can be prepared as outlined in Scheme 4. Alcohols of formula (V) can be treated with a suitable dihaloaryl compound of formula (VIII) under standard SN_{Ar} conditions, such as DBU and DMSO at 120° C.

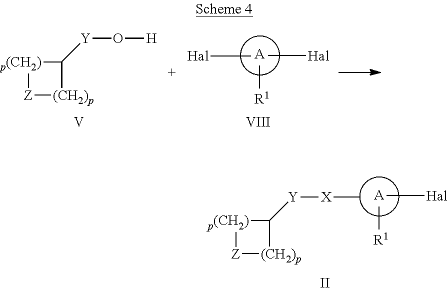

Building blocks of formula (II) where X is NR⁷ can be prepared as outlined in Scheme 5. Amines of formula (IX) can be treated with a suitable dihaloaryl compound of formula (VIII) under standard SN_{Ar} conditions, such as DBU and DMSO at 120° C.

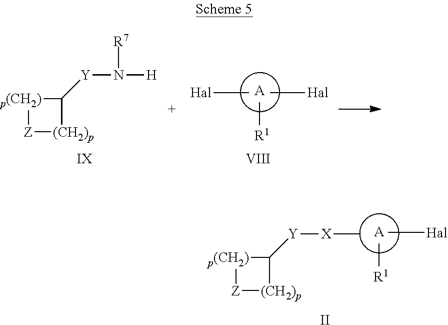

Alternatively, building blocks of formula (II) where X is NR⁷ can be prepared as outlined in Scheme 6. Mesylates of formula (VII) can be treated with amines of formula (X) under standard conditions, for example, NaH in DMF at room temperature.

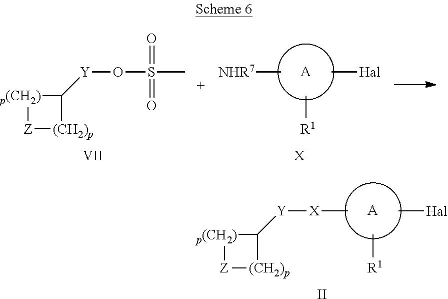

Building blocks of formula (II) where X is CR⁶R^{66,} Y is C₂₋₄ alkylene and both R⁶ and R⁶⁶ are hydrogen can be prepared as outlined in Scheme 7. An alkyne of formula (XII) can be prepared from an alcohol of formula (V) by oxidation to the corresponding aldehyde (XI) using a standard oxidizing reagent, such as Dess-Martin Periodinane, and subsequent reaction of the aldehyde of formula (XI) with trimethylsilyldiazomethane, which has previously been treated with a suitable base, such as nBuLi. Alkynes of formula (XIII) can be prepared by reaction of alkynes of formula (XII) with dihaloaryl compounds of formula (VIII) under standard Sonogashira coupling conditions. Compounds of formula (II) as described above can be prepared from alkynes of formula (XIII) under standard reduction conditions, such as 10% palladium on carbon under an atmosphere of hydrogen in a suitable solvent such as methanol.

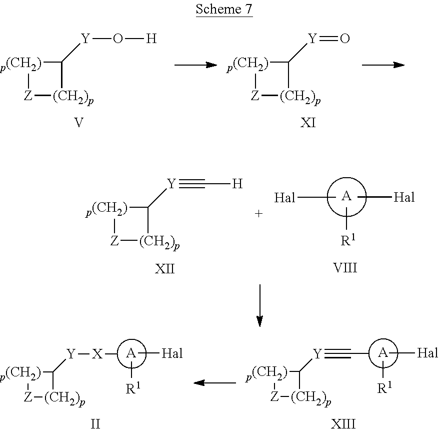

Building blocks of formula (II) where X is CR⁶R⁶⁶ and Y is (CH₂)ₛO, where s is 2 or 3 and R⁶ and R⁶⁶ are not fluorine can be prepared as outlined in Scheme 8. Mesylates of formula (XIV) can be prepared from alcohols of formula (XV) under standard conditions, such as, methansulfonyl chloride and triethylamine in DCM. Compounds of formula (II) as described above can be prepared by reaction of mesylates of formula (XIV) with alcohols of formula (XVI) under standard conditions, such as NaH in DMF at room temperature.

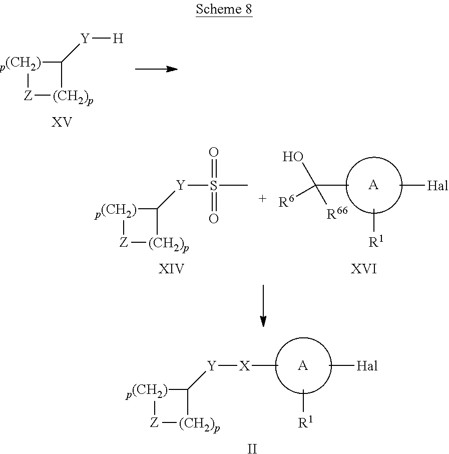

Building blocks of formula (II) where X is CR⁶R⁶⁶ and Y is (CH₂)ₛO(CH₂)ₜ, where s and t are 1 or 2 but the sum of s and t is not >3, can be prepared as outlined in Scheme 9. Mesylates of formula (XVIII) can be prepared from alcohols of formula (XVII) under standard conditions, such as, methanesulfonyl chloride and triethylamine in DCM. Building blocks of formula (II) as described above can be prepared by reaction of mesylates of formula (XVIII) with alcohols of formula (XIX) under standard conditions, such as NaH in DMF at room temperature.

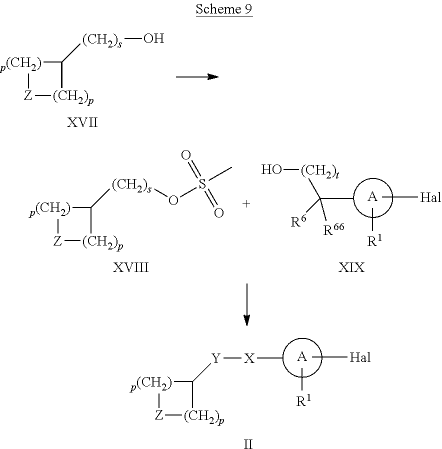

Building blocks of formula (II) where X is CR⁶R⁶⁶ and Y is O(CH₂)ₛ, where R⁶ and R⁶⁶ are both hydrogen and s is 2 or 3, can be prepared as outlined in Scheme 10. Alkynes of formula (XXI) can be prepared from alcohols of formula (XX) and a suitable alkylating agent, for example propargyl bromide, under standard conditions, such as NaH in DMF at room temperature. Alkynes of formula (XXII) can be prepared by reaction of alkynes of formula (XXI) with dihaloaryl compounds of formula (VIII) under standard Sonogashira coupling conditions. Compounds of formula (II) as described above can be prepared from alkynes of formula (XXII) under standard reduction conditions, such as 10% palladium on carbon under an atmosphere of hydrogen in a suitable solvent such as methanol.

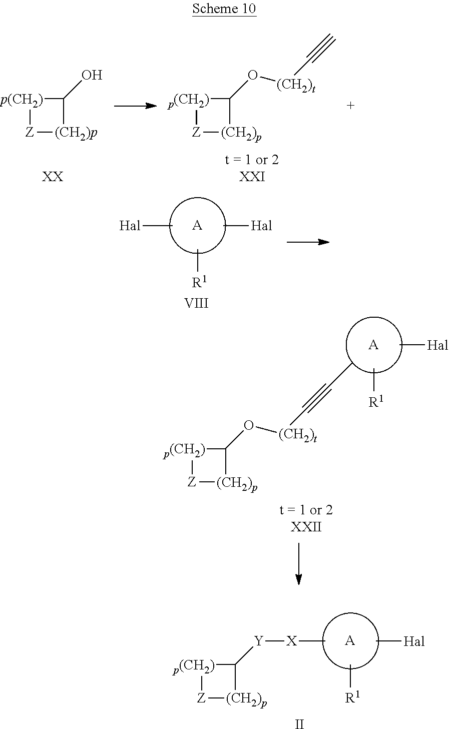

Building blocks of formula (II) where X is CR⁶R⁶⁶ and Y is C₃₋₄ alkylene or O(CH₂)ₛ, where R⁶ is alkyl, R⁶⁶ is hydrogen and s is 2 or 3, can be prepared as outlined in Scheme 11. Phosphonium bromides of formula (XXIV) can be prepared from alkyl bromides of formula (XXIII) by treatment with triphenylphosphine in THF. Alkenes of formula (XXVI) can be prepared by reaction phosphonium bromides of formula (XXIV) with ketones of formula (XXV) under standard Wittig conditions. Compounds of formula (II) as described above can be prepared from alkenes of formula (XXVI) under standard reduction conditions, such as 10% palladium on carbon under an atmosphere of hydrogen in a suitable solvent such as methanol.

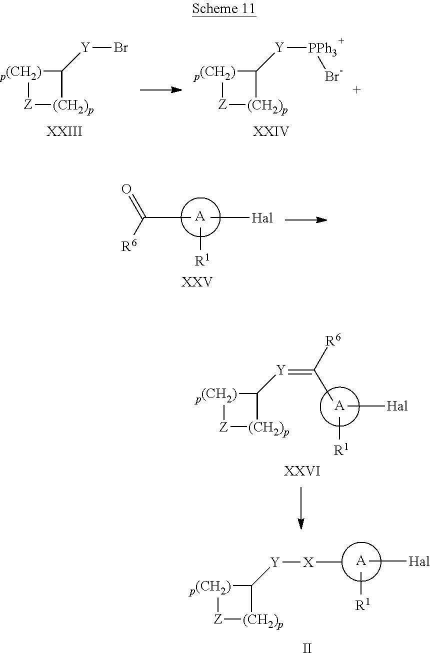

Building blocks of formula (II) where X is CR⁶R⁶⁶ and Y is C₂₋₄ alkylene, where R⁶ and R⁶⁶ are fluorine, can be prepared as outlined in Scheme 12. Ketones of formula (XXVII) can be prepared from alkynes of formula (XIII) by treatment with mercury oxide and sulphuric acid in methanol/water at 80° C. Compounds of formula (II) as described above can be prepared from ketones of formula (XXVII) under standard conditions, for example, diethylaminosulfur trifluoride in a suitable solvent, such as DCM.

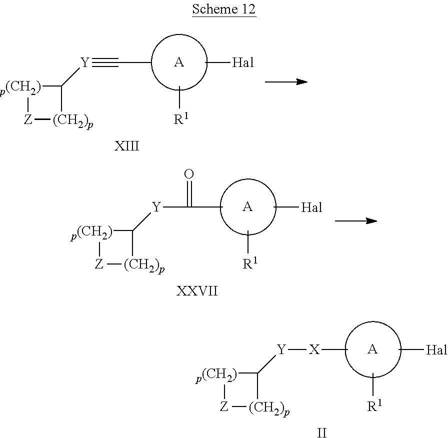

Building blocks of formula (II) where X is CR⁶R⁶⁶ and Y is O(CH₂)ₛ, where R⁶ and R⁶⁶ are fluorine and s is 2 or 3, can be prepared as outlined in Scheme 13. Ketones of formula (XXVIII) can be prepared from alkynes of formula (XXII) by treatment with mercury oxide and sulphuric acid in methanol/water at 80° C. Compounds of formula (II) as described above can be prepared from ketones of formula (XXVIII) under standard conditions, for example, diethylaminosulfur trifluoride in a suitable solvent, such as DCM.

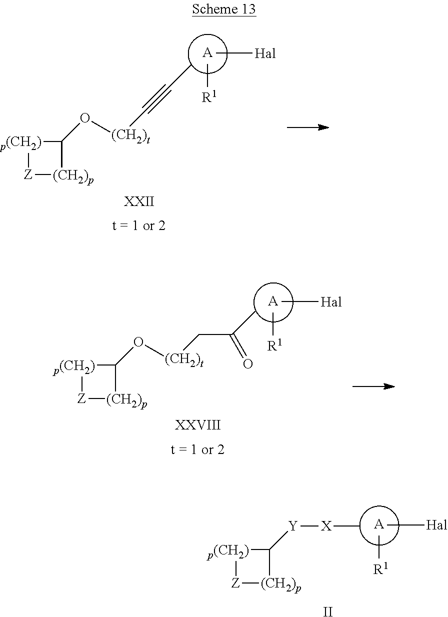

Building blocks of formula (II) where X is CR⁶R⁶⁶ and Y is C₂₋₄ alkylene, where R⁶ is fluorine and R⁶⁶ is hydrogen can be prepared as outlined in Scheme 14. Alcohols of formula (XXIX) can be prepared from ketones of formula (XXVII) under standard conditions, for example, sodium borohydride in methanol. Compounds of formula (II) as described above can be prepared from alcohols of formula (XXIX) under standard conditions, for example, diethylaminosulfur trifluoride in a suitable solvent, such as DCM.

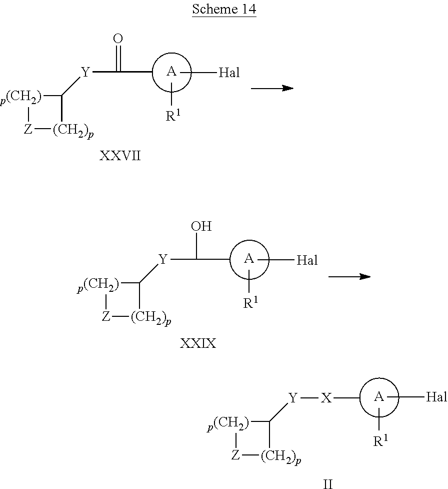

Building blocks of formula (II) where X is CR⁶R⁶⁶ and Y is O(CH₂)ₛ, where R⁶ is fluorine, R⁶⁶ is hydrogen and s is 2 or 3, can be prepared as outlined in Scheme 15. Alcohols of formula (XXX) can be prepared from ketones of formula (XXVIII) under standard conditions, for example, sodium borohydride in methanol. Compounds of formula (II) as described above can be prepared from alcohols of formula (XXX) under standard conditions, for example, diethylaminosulfur trifluoride in a suitable solvent, such as DCM.

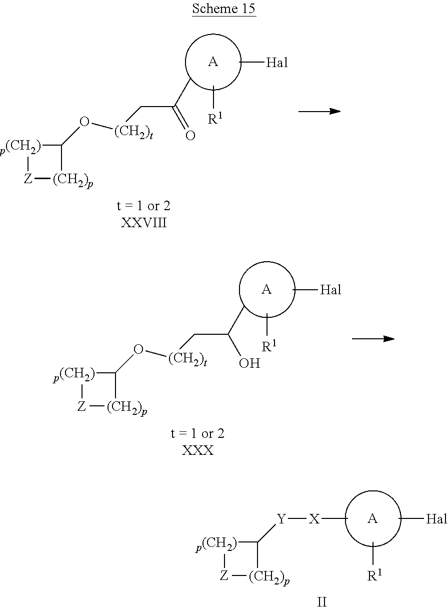

Examples and syntheses of building blocks of formula (III) have been described elsewhere: Benbow et.al., WO2007/148185; Brackes et.al., Bioorg. Med. Chem. Lett., 2007, 17 2005-2012; Pei et.al., J. Med. Chem., 2007, 50 (8), 1983-1987; Cox et.al., Bioorg. Med. Chem. Lett., 2007, 17 4579-4583; Wright et.al., Bioorg. Med. Chem. Lett., 2007, 17 5638-5642.

The syntheses of building blocks of formulae (V), (IX), (XV), (XVII), (XX) and (XXIII) have been described elsewhere: Bertram et.al., WO2008/081204; Fang et. al., WO2008/070692; Ma et.al., WO2009/014910; Alper et. al., WO2008/097428.

Other compounds of formula (I) may be prepared by methods analogous to those described above or by methods known per se. Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

The processes for the production of the compounds of formula (I) and intermediates thereto as described above are also included as further aspects of the present invention.

Any novel intermediates as defined in the Schemes above or in the Examples, are also included within the scope of the invention. Therefore according to a further aspect of the invention there is provided a compound of any one of formulae (II), (IV), (XIII), (XXII), (XXVI), (XXVII), (XXVIII), (XXIX), (XXVIII) and (XXX) as defined above. The preferred groups for variables recited above in relation to the compounds of formula (I) also apply to the intermediates compounds.

As indicated above the compounds of the invention are useful as GPR119 agonists, e.g. for the treatment and/or prophylaxis of diabetes. For such use the compounds of the invention will generally be administered in the form of a pharmaceutical composition.

The compounds of the invention may also be useful as dual GPR119 agonists/DPP-IV inhibitors, e.g. for the treatment and/or prophylaxis of diabetes. For such use the compounds of the invention will generally be administered in the form of a pharmaceutical composition.

The invention also provides a compound of the invention, or a pharmaceutically acceptable salt thereof, for use as a pharmaceutical.

The invention also provides a pharmaceutical composition comprising a compound of the invention, in combination with a pharmaceutically acceptable carrier.

Preferably the composition is comprised of a pharmaceutically acceptable carrier and a non-toxic therapeutically effective amount of a compound of the invention, or a pharmaceutically acceptable salt thereof.

Moreover, the invention also provides a pharmaceutical composition for the treatment of disease by modulating GPR119 and optionally DPP-IV, resulting in the prophylactic or therapeutic treatment of diabetes, comprising a pharmaceutically acceptable carrier and a non-toxic therapeutically effective amount of compound of the invention, or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of the invention, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of the invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of the invention may be used in the treatment of diseases or conditions in which GPR119 and optionally DPP-IV play a role.

Thus the invention also provides a method for the treatment of a disease or condition in which GPR119 and optionally DPP-IV play a role comprising a step of administering to a subject in need thereof an effective amount of a compound of the invention, or a pharmaceutically acceptable salt thereof. Such diseases or conditions diabetes, obesity, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The invention also provides a method for the treatment of type II diabetes, comprising a step of administering to a patient in need thereof an effective amount of a compound of the invention, or a pharmaceutically acceptable salt thereof.

The invention also provides a method for the treatment of obesity, metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of the invention, or a pharmaceutically acceptable salt thereof.

The invention also provides a compound of the invention, or a pharmaceutically acceptable salt thereof, for use in the treatment of a condition as defined above.

The invention also provides the use of a compound of the invention, or a pharmaceutically acceptable salt thereof, in the manufacture of a medicament for the treatment of a condition as defined above.

In the methods of the invention the term “treatment” includes both therapeutic and prophylactic treatment.

The compounds of the invention may exhibit advantageous properties compared to known compounds or combination therapies for the treatment of diabetes.

The compounds of the invention, or pharmaceutically acceptable salts thereof, may be administered alone or in combination with one or more other therapeutically active compounds. The other therapeutically active compounds may be for the treatment of the same disease or condition as the compounds of the invention or a different disease or condition. The therapeutically active compounds may be administered simultaneously, sequentially or separately.

The compounds of the invention may be administered with other active compounds for the treatment of obesity and/or diabetes, for example insulin and insulin analogs, gastric lipase inhibitors, pancreatic lipase inhibitors, sulfonyl ureas and analogs, biguanides e.g. metformin, α2 agonists, glitazones, PPAR-γ agonists, mixed PPAR-α/γ agonists, RXR agonists, fatty acid oxidation inhibitors, α-glucosidase inhibitors, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of the invention, or a pharmaceutically acceptable salt thereof, and at least one other agent, for example another agent for the treatment of diabetes or obesity, represents a further aspect of the invention.

The present invention also provides a method for the treatment of diabetes in a mammal, such as a human, which method comprises administering an effective amount of a compound of the invention, or a pharmaceutically acceptable salt thereof, and another agent, for example another agent for the treatment of diabetes or obesity, to a mammal in need thereof.

The invention also provides the use of a compound of the invention, or a pharmaceutically acceptable salt thereof, and another agent for the treatment of diabetes.

The invention also provides the use of a compound of the invention, or a pharmaceutically acceptable salt thereof, in the manufacture of a medicament for use in combination with another agent, for the treatment of diabetes.

The compound of the invention, or a pharmaceutically acceptable salt thereof, and the other agent(s) may be co-administered or administered sequentially or separately.

Co-administration includes administration of a formulation which includes both the compound of the invention, or a pharmaceutically acceptable salt thereof, and the other agent(s), or the simultaneous or separate administration of different formulations of each agent. Where the pharmacological profiles of the compound of the invention, or a pharmaceutically acceptable salt thereof, and the other agent(s) allow it, coadministration of the two agents may be preferred.

The invention also provides the use of a compound of the invention, or a pharmaceutically acceptable salt thereof, and another agent in the manufacture of a medicament for the treatment of diabetes.

The invention also provides a pharmaceutical composition comprising a compound of the invention, or a pharmaceutically acceptable salt thereof, and another antidiabetic agent, and a pharmaceutically acceptable carrier.

The invention also encompasses the use of such compositions in the methods described above.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

Materials and methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O-MeCN solution containing 0.1% HCO₂H over 6 min with UV detection at 220 nm. Gradient information: 0.0-0.3 min 100% H₂O; 0.3-4.25 min: Ramp up to 10% H₂O-90% MeCN; 4.25-4.4 min: Ramp up to 100% MeCN; 4.4-4.9 min: Hold at 100% MeCN; 4.9-6.0 min: Return to 100% H₂O. The mass spectra were obtained using an electrospray ionisation source in either the positive (ES⁺) or negative (ES⁻) ion modes.

LCMS-method 2 data were obtained as follows: Xbridge C18 column (2.1×50 mm, 2.5 μM, flow rate 0.8 mL/min) eluting with an MeCN-10 mM NH₄HCO₃ solution over 1.5 min with UV detection at 215-350 nm. Gradient information: 0-0.8 min: 98% MeCN 2% NH₄HCO₃ to 98% NH₄HCO₃ 2% MeCN; 0.8-1.2 min: hold at 98% NH₄HCO₃ 2% MeCN. The mass spectra were obtained using an electrospray ionisation source in the positive (ES⁺) mode.

LCMS-method 3 data were obtained as follows: Xbridge C18 column (2.1×5.0 mm, 2.55 μM, flow rate 0.8 mL/min) eluting with an MeCN-10 mM NH₄HCO₃ solution over 5 min with UV detection at 215-350 nm. Gradient information: 0-4 min: 98% MeCN 2% NH₄HCO₃ to 98% NH₄HCO₃ 2% MeCN; 4-4.6 min: hold at 98% NH₄HCO₃ 2% MeCN. The mass spectra were obtained using an electrospray ionisation source in the positive (ES⁺) mode.

LCMS-method 4 data were obtained as follows: Xbridge C18 column (3.0×150 mm, 5 μM, flow rate 1.0 mL/min) eluting with an MeCN-10 mM NH₄HCO₃ solution over 5 min with UV detection at 215-350 nm. Gradient information: 0-0.1 min: hold at 5% MeCN 95% NH₄HCO₃; 0.1-3.0 min: 5% MeCN 95% NH₄HCO₃ to 5% NH₄HCO₃ 95% MeCN; 3.0-3.9 min: hold at 5% NH₄HCO₃ 95% MeCN. The mass spectra were obtained using an electrospray ionisation source in the positive (ES⁺) mode.

Chiral-HPLC was performed on a Daicel chiralpak IA 250×20 mm, 5 μM column.

Abbreviations and acronyms: AcOH: Acetic acid; ADDP: Azodicarboxylic dipiperidide; BA: n-Butylamine; CHCl₃: Chloroform; DBU: 1,8-Diazabicyclo[5.4.0]undec-7-ene; DCM: Dichloromethane; DEA: Diethylamine; DIPE: Diisopropyl ether; DMAP: Dimethylpyridin-4-ylamine; DMF: Dimethylformamide; DMSO: Dimethylsulfoxide; EDCI: (3-Dimethylaminopropyl)ethylcarbodiimide hydrochloride; Et₂O: Diethyl ether; EtOH: Ethanol; EtOAc: Ethyl Acetate; h: hour(s); HCl: Hydrochloric acid; HCO₂H: Formic acid; H₂O: Water; HOBt: 1-Hydroxybenzotriazole monohydrate; HPLC: High performance liquid chromatography; IH: Isohexane; IMS: Industrial methylated spirit; IPA: Isopropyl alcohol; LAH: Lithium aluminium hydride; M: Molar; MeCN: Acetonitrile; MeOH: Methanol; MgSO4: Magnesium sulfate; min: minute/s; MTBE: Methyl-tert-butyl ether; Na₂CO₃: Sodium carbonate; NaHCO₃: Sodium hydrogen carbonate; NaOH: Sodium hydroxide; Na₂SO₄: Sodium sulfate; NH₄Cl: Ammonium chloride; NH₄HCO₃: Ammonium bicarbonate; NH₄OH: Ammonium hydroxide; Pd: Palladium; RT: Retention time; r.t.: Room temperature; sat: saturated; SCX: Strong Cation Exchange resin; SiO₂: Silica gel; THF: Tetrahydrofuran; TFA: Trifluoroacetic acid; TsOH: p-Toluenesulfonic acid monohydrate

The syntheses of the following compounds have been described elsewhere: (3S,4S)-3,4-Diazido-1-benzylpyrrolidine and 5-bromo-4,4-difluoropentanoic acid: Benbow et. al., WO2007/148185; tert-Butyl 4-((R)-3-hydroxy-1-methylpropyl)piperidine-1-carboxylate, (R)-3-piperidin-4-yl butan-1-ol hydrochloride and (R)-3-[1-(3-isopropyl-[1,2,4]oxadiazol-5-yl)piperidin-4-yl]butan-1-ol: Fyfe et. al., WO2008/081204; 3-[1-(5-Isopropyl-[1,2,4]oxadiazol-3-yl)piperidin-4-yl]propan-1-ol: Fyfe et. al., WO2008/081206; 2-Bromo-5-{3-[1-(3-isopropyl-[1,2,4]oxadiazol-5-yl)piperidin-4-yl]propoxy}pyridine: Fyfe et. al., WO2008/081208; Methanesulfonic acid 3-[1-(3-isopropyl-[1,2,4]oxadiazol-5-yl)piperidin-4-yl]propyl ester and (R)-3-[1-(3-ethyl-[1,2,4]oxadiazol-5-yl)piperidin-4-yl]butan-1-ol: Barba et. al., WO2009/034388.

3-Piperidin-4-yl-propan-1-ol hydrochloride was purchased from Sigma-Aldrich. All other compounds were available from commercial sources.

### Preparation 1: (R)-3-[1-(5-Chloropyrimidin-2-yl)piperidin-4-yl]-butan-1-ol

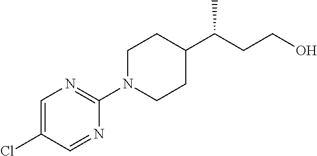

TFA (75 mL) was added to a solution of tert-butyl 4-((R)-3-hydroxy-1-methylpropyl)piperidine-1-carboxylate (30.0 g, 117 mmol) in DCM (150 mL) at 0° C. and the resulting solution was stirred at this temperature for 0.5 h. The solvent was removed in vacuo and the remainder dissolved in DCM then washed with saturated aqueous NaHCO₃ solution, dried (MgSO₄), filtered and concentrated in vacuo to afford (R)-3-piperidin-4-ylbutan-1-ol. To a portion of this material (10.0 g, 63.7 mmol) in DMSO (65 mL) was added DBU (14.3 mL, 95.5 mmol) and 2,5-dichloropyrimidine (14.3 g, 95.5 mmol) and the resulting reaction mixture was heated at 100° C. for 1.5 h. The reaction mixture was cooled to ambient temperature, quenched with water and extracted with EtOAc. The organic extracts were washed with 1M HCl solution and brine, dried (MgSO₄), filtered and concentrated in vacuo. Purification by column chromatography (EtOAc:IH, 1:4 to 7:13) afforded the title compound: RT=3.58 min, m/z (ES⁺)=270.1 [M+H]⁺.

### Preparation 2: (R)-Methanesulfonic acid-3-[1-(5-chloropyrimidin-2-yl)piperidin-4-yl]butyl ester

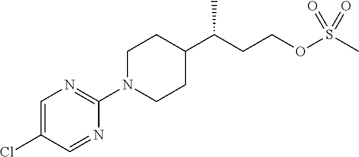

A solution of (R)-3-[1-(5-chloropyrimidin-2-yl)piperidin-4-yl]-butan-1-ol (Preparation 1, 500 mg, 1.86 mmol), and triethylamine (777 μL, 5.58 mmol) in DCM (15 mL) was cooled to 0° C. Methanesulfonyl chloride (288 μL, 3.72 mmol) was added and the mixture was stirred at 0° C. for 20 min until complete. The reaction was diluted with water and organics were extracted into DCM (×2) and passed through a phase separater. Removal of the solvent in vacuo afforded the title compound: RT=4.10 min, m/z (ES⁺)=348.1 [M+H]⁺.

### Preparation 3: (R)-Methanesulfonic acid-3-[1-(3-isopropyl-[1,2,4]oxadiazol-5-yl)piperidin-4-yl]butyl ester

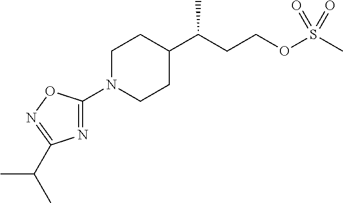

Methanesulfonyl chloride (610 μL, 7.90 mmol) and triethylamine (2.01 mL, 15.0 mmol) were added to a solution of (R)-3-[1-(3-isopropyl-[1,2,4]oxadiazol-5-yl)piperidin-4-yl]butan-1-ol (2.00 g, 7.50 mmol) in DCM (30 mL) at 0° C. After stirring for 10 min, the reaction was diluted with DCM (100 mL) and poured into saturated aqueous NaHCO₃ solution (100 mL). The organic layer was separated, washed with 0.1M HCl (100 mL), dried (MgSO₄), filtered and concentrated in vacuo. Purification by column chromatography (EtOAc:IH, 1:1) afforded the title compound: RT=3.42 min; m/z (ES⁺)=346.1 [M+H]⁺.

### Preparation 4: (R)-5-Chloro-2-{4-[1-methyl-3-(2-chloropyrimidin-5-yloxy)propyl]-piperidin-1-yl}pyrimidine

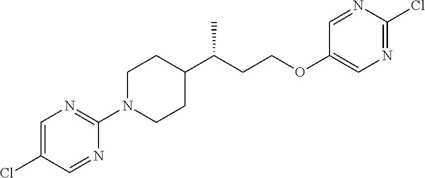

A combination of (R)-methanesulfonic acid-3-[1-(5-chloropyrimidin-2-yl)piperidin-4-yl]butyl ester (Preparation 2, 641 mg, 1.85 mmol), 2-chloro-5-hydroxypyrimidine (480 mg, 3.69 mmol) and potassium carbonate (510 mg, 3.69 mmol) in DMF (4 mL) were heated at 80° C. until the reaction was complete. The mixture was diluted with water and extracted into EtOAc (×3), then the organic fractions were combined, washed with 1M NaOH solution, brine, and dried (MgSO₄). Removal of the solvent in vacuo afforded the title compound: RT=4.72 min; m/z (ES⁺)=382.1 [M+H]⁺.

### Preparation 5: (R)-2-Bromo-5-{3-[1-(3-isopropyl-[1,2,4]oxadiazol-5-yl)piperidin-4-yl]butoxy}pyridine

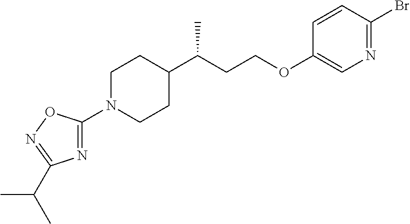

A mixture of (R)-3-[1-(3-isopropyl-[1,2,4]oxadiazol-5-yl)piperidin-4-yl]butan-1-ol (500 mg, 1.87 mmol), 2-bromo-5-hydroxypyridine (32.5 mg, 1.87 mmol) and tri n-butyl phosphine (930 μL, 3.74 mmol) in dry toluene (25 mL) was cooled to 0° C. and a solution of ADDP (942 mg, 3.74 mmol) in toluene (25 mL) was added, dropwise. The reaction was allowed to reach r.t. and stirred for a further 72 h before adding iso-hexane (100 mL) and stirring vigorously for 30 min. The mixture was then filtered, and the filtrate concentrated in vacuo. The crude residue was dissolved in EtOAc and washed with 0.1M NaOH solution, 0.1M citric acid then brine, and dried (MgSO₄). Removal of the solvent in vacuo followed by purification by column chromatography (IH:EtOAc, 1:1) afforded the title compound: RT=4.87 min; m/z (ES⁺)=425.3 [M+H]⁺.

### Preparation 6: (R)-4-Chloro-6-{3-[1-(3-isopropyl-[1,2,4]oxadiazol-5-yl)piperidin-4-yl]butoxy}pyrimidine

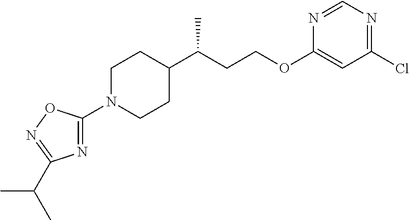

To a dry solution of (R)-3-[1-(3-isopropyl-[1,2,4]oxadiazol-5-yl)piperidin-4-yl]butan-1-ol (1.48 g, 5.54 mmol) in THF (25 mL), cooled to 0° C., was added sodium hydride (60% in mineral oil, 252 mg, 6.29 mmol) and the reaction was allowed to warm to r.t. over 15 min. A solution of 4,6-dichloropyrimidine (750 mg, 5.03 mmol) in THF was added dropwise, over 5 min and the resulting reaction was heated to 60° C. for 16 h. The crude mixture was partitioned between EtOAc and brine and then the organic phase was separated and dried (MgSO₄). Removal of the solvent in vacuo followed by purification by column chromatography (IH:EtOAc, 100:0, 90:10, 80:20, 70:30) afforded the title compound: RT=4.05 min; m/z (ES⁺)=380.3 [M+H]⁺.

### Preparation 7: (R)-2-Chloro-5-{3-[1-(3-isopropyl-[1,2,4]oxadiazol-5-yl)piperidin-4-yl]butoxy}pyrimidine

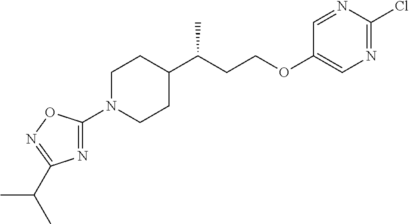

A combination of (R)-methanesulfonic acid-3-[1-(3-isopropyl-[1,2,4]oxadiazol-5-yl)piperidin-4-yl]butyl ester (Preparation 3, 560 mg, 1.62 mmol), 2-chloro-5-hydroxypyrimidine (423 mg, 3.24 mmol) and potassium carbonate (447 mg, 3.24 mmol) in DMF (4 mL) was heated to 70° C. for 24 h. The reaction mixture was diluted with water (75 mL) and extracted with EtOAc (2×75 mL). The combined organic fractions were washed with 1M NaOH solution, then brine, and dried (MgSO₄). Removal of the solvent in vacuo afforded the title compound: RT=4.14 min; m/z (ES⁺)=380.1 [M+H]⁺.

### Preparation 8: 2-Chloro-5-methoxy-4-methylpyrimidine

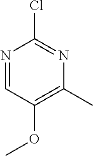

To a solution of 2,4-dichloro-5-methoxypyrimidine (1.5 g, 8.38 mmol) in THF (15 mL) under argon was added trimethylboroxine (1.2 g, 9.22 mmol), dichlorobis(triphenylphosphine)-palladium (0.6 g, 0.84 mmol) and potassium phosphate (3.6 g, 16.76 mmol) and the mixture was heated to reflux for 16 h. The reaction was diluted with EtOAc and washed with water, then dried (Na₂SO₄). Removal of the solvent in vacuo followed by purification by column chromatography (IH:EtOAc, 100:0, 90:10, 80:20, 70:30) afforded the title compound: RT=2.45 min; m/z (ES⁺)=159.0 [M+H]⁺.

### Preparation 9: 2-Chloro-4-methylpyrimidin-5-ol

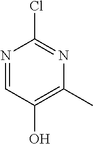

To a dry solution of 2-chloro-5-methoxy-4-methylpyrimidine (Preparation 8, 1.1 g, 6.94 mmol) in DCM (20 mL) under argon, cooled to −78° C. was added boron tribromide (2.63 mL, 27.82 mmol), dropwise over 15 min. The reaction was allowed to stir at −78° C. for 20 min then at r.t. for 16 h. The mixture was cooled down to −78° C. and then quenched by the dropwise addition of MeOH. The reaction was allowed to reach r.t. then NaHCO₃ was carefully added to adjust the pH to 5. Organics were extracted into EtOAc, washed with brine and dried (Na₂SO₄). Removal of the solvent in vacuo followed by trituration with DCM afforded the title compound: RT=2.13 min; m/z (ES⁺)=145.0 [M+H]⁺.

### Preparation 10: (R)-2-Chloro-5-{3-[1-(3-isopropyl-[1,2,4]oxadiazol-5-yl)piperidin-4-yl]butoxy}-4-methylpyrimidine

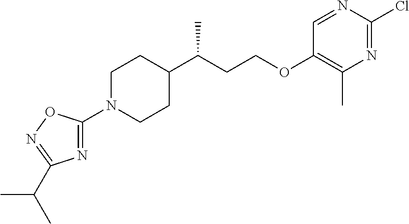

A combination of (R)-methanesulfonic acid-3-[1-(3-isopropyl-[1,2,4]oxadiazol-5-yl)piperidin-4-yl]butyl ester (Preparation 3, 250 mg, 0.72 mmol), 2-chloro-4-methylpyrimidin-5-ol (Preparation 9, 110 mg, 0.76 mmol) and potassium carbonate (200 mg, 1.45 mmol) in DMF (4 mL) were heated at 80° C. for 2 h. The mixture was allowed to cool to r.t. and partitioned between EtOAc and brine. The organic phase was washed with brine, then sat. Na₂CO₃ solution, and dried (Na₂SO₄). Removal of the solvent in vacuo afforded the title compound: RT=4.22 min; m/z (ES⁺)=394.2 [M+H]⁺.

### Preparation 11: 5-Bromo-2-{(R)-3-[1-(3-isopropyl-[1,2,4]oxadiazol-5-yl)piperidin-4-yl]butoxy}pyridine

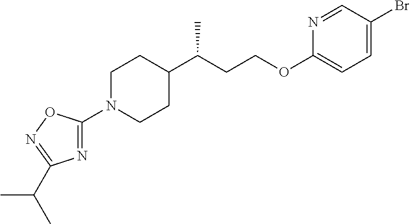

A solution of 5-bromo-2-pyridinol (390 mg, 2.24 mmol) and (R)-3-[1-(3-isopropyl-[1,2,4]oxadiazol-5-yl)piperidin-4-yl]butan-1-ol (400 mg, 1.50 mmol) in THF (14 mL) was treated with diethyl azodicarboxylate (283 μL, 1.80 mmol) and triphenylphosphine (471 mg, 1.80 mmol) and the mixture was stirred at r.t. for 36 h. After this time further portions of diethyl azodicarboxylate (283 μL, 1.80 mmol) and triphenylphosphine (471 mg, 1.80 mmol) were added and stirring continued until the reaction was complete. The reaction was quenched with MeOH (1 mL) then the solvent was concentrated in vacuo. The resulting residue was dissolved in DCM and washed with 3M NaOH solution, 1M HCl solution, brine, then dried (MgSO₄). Removal of the solvent in vacuo and purification by column chromatography (IH:EtOAc, 85:15, 70:30) afforded the title compound: RT=4.42 min; m/z (ES⁺)=423.2, 425.4 [M+H]⁺.

### Preparation 12: (R)-5-Bromo-2-{3-[1-(3-isopropyl-[1,2,4]oxadiazol-5-yl)piperidin-4-yl]butoxy}pyrimidine

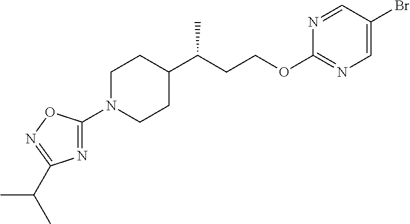

A solution of 5-bromo-2-hydroxypyrimidine (196 mg, 1.12 mmol) and (R)-3-[1-(3-isopropyl-[1,2,4]oxadiazol-5-yl)piperidin-4-yl]butan-1-ol (200 mg, 0.75 mmol) in THF (7 mL) was treated with diethyl azodicarboxylate (141 μL, 0.9 mmol) and triphenylphosphine (235 mg, 0.9 mmol) and the mixture was stirred at r.t. until complete. The reaction was quenched with MeOH (1 mL) and the solvent was concentrated in vacuo. The resulting residue was dissolved in DCM and washed with 3M NaOH solution, 1M HCl solution, brine and dried (MgSO₄). Removal of the solvent in vacuo followed by purification by column chromatography (IH:EtOAc, 70:30) afforded the title compound: RT=4.16 min; m/z (ES⁺)=424.2, 426.2 [M+H]⁺.

The following compounds were prepared by reacting (R)-3-[1-(3-isopropyl-[1,2,4]oxadiazol-5-yl)piperidin-4-yl]butan-1-ol with the appropriate halogenated heterocyclic building block, employing the procedure outlined in Preparation 12:

| Prep | | | |
|---|---|---|---|
| No. | Structure | Name | LCMS |
| 13 | 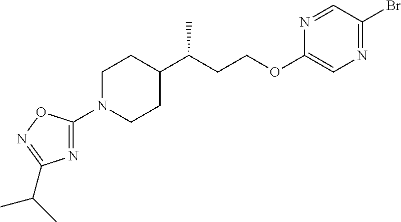 | (R)-2-Bromo-5-{3-[1- (3-isopropyl- [1,2,4]oxadiazol-5- yl)piperidin-4- yl]butoxy}pyrazine | RT = 4.50 min; m/z (ES⁺) = 424.1, 426.1 [M + H]⁺. |
| 14 | 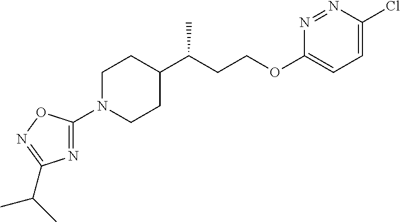 | 3-Chloro-6-{(R)-3-[1- (3-isopropyl- [1,2,4]oxadiazol-5- yl)piperidin-4- yl]butoxy}pyridazine | RT = 3.59 min; m/z (ES⁺) = 380.2 [M + H]⁺. |

### Preparation 15: 4-(R)-3-Hydroxy-1-methylpropyl)piperidine-1-carboxylic acid isopropyl ester

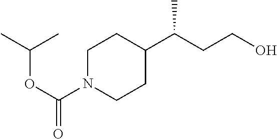

To a suspension of (R)-3-piperidin-4-yl butan-1-ol hydrochloride (2.5 g, 12.91 mmol) in DCM (150 mL), under argon, was added triethylamine (5.4 mL, 38.72 mmol) and the reaction was stirred for 30 min. The mixture was cooled to 0° C. and a solution of iso-propyl chloroformate in toluene (1M, 15.49 mmol, 15.49 mL) was added, dropwise, over 1 hr before allowing the mixture to warm to r.t. over 16 h. Sat. NaHCO₃ solution was added and the mixture was stirred vigorously for 20 min. The organic portion was separated, washed with sat. NaHCO₃ solution, dilute citric acid, and dried (Na₂SO₄). Removal of the solvent in vacuo afforded the title compound: RT=3.59 min; m/z (ES⁺)=244.2 [M+H]⁺.

### Preparation 16: 4-((R)-3-Methanesulfonyloxy-1-methylpropyl)piperidine-1-carboxylic acid isopropyl ester

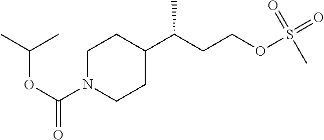

To a solution of 4-(R)-3-hydroxy-1-methylpropyl)piperidine-1-carboxylic acid isopropyl ester (Preparation 15, 3.30 g, 13.56 mmol) in DCM (50 mL) was added triethylamine (4.72 mL, 33.90 mmol) and the reaction cooled to 0° C. A solution of methanesulfonyl chloride (1.25 mL, 16.27 mmol) in DCM (10 mL) was added over 10 min and the resulting mixture was stirred at 0° C. until complete. The reaction was quenched with sat. NaHCO₃ solution, then the organic phase was separated, washed with citric acid solution (0.5M), brine, and dried (Na₂SO₄). Removal of the solvent in vacuo afforded the title compound: RT=3.58 min; m/z (ES⁺)=322.2 [M+H]⁺.

### Preparation 17: 4-[(R)-3-(2-Chloropyrimidin-5-yloxy)-1-methylpropyl]piperidine-1-carboxylic acid isopropyl ester

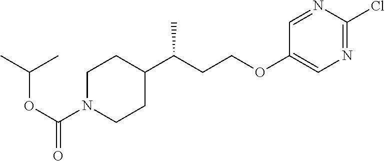

A combination of 4-((R)-3-methanesulfonyloxy-1-methylpropyl)piperidine-1-carboxylic acid isopropyl ester (Preparation 16, 300 mg, 1.56 mmol), 2-chloro-5-hydroxypyrimidine (213 mg, 1.63 mmol) and potassium carbonate (430 mg, 3.11 mmol) in DMF (5.0 mL) were heated in a sealed tube to 70° C. for 72 h. The reaction mixture was partitioned between EtOAc and brine and organic phase separated and dried (Na₂SO₄). Removal of the solvent in vacuo afforded the title compound: RT=4.14 min m/z (ES⁺)=356.1 [M+H]⁺.

### Preparation 18: 2-Bromo-5-{(R)-3-[1-(3-isopropyl-[1,2,4]oxadiazol-5-yl)piperidin-4-yl]butoxy}-3-methylpyridine

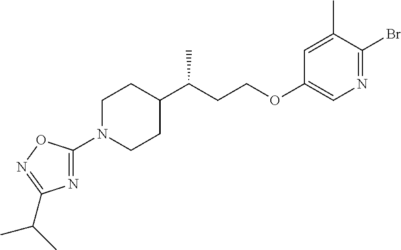

To a solution of (R)-methanesulfonic acid-3-[1-(3-isopropyl-[1,2,4]oxadiazol-5-yl)piperidin-4-yl]butyl ester (Preparation 3, 250 mg, 0.72 mmol) and 6-bromo-5-methylpyridin-3-ol (143 mg, 0.76 mmol) in DMF (5.0 mL), under argon, was added potassium carbonate (200 mg, 1.45 mmol) and the mixture was heated in a sealed tube at 70° C. for 16 h. The reaction was diluted with EtOAc and the resulting solution was washed with brine (2×50 mL), sat. NaHCO₃ solution and water, then dried (Na₂SO₄). Removal of the solvent in vacuo afforded the title compound: RT=4.60 min; m/z (ES⁺)=439.1 [M+H]⁺.

### Preparation 19: 2-Chloro-5-{3-[1-(3-isopropyl-[1,2,4]oxadiazol-5-yl)piperidin-4-yl]propoxy}pyrimidine

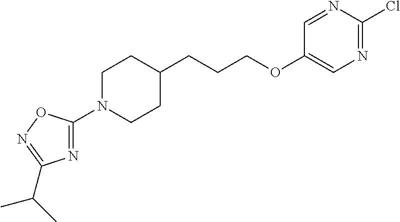

The title compound was prepared from methanesulfonic acid 3-[1-(3-isopropyl-[1,2,4]oxadiazol-5-yl)piperidin-4-yl]propyl ester and 2-chloro-5-hydroxypyrimidine employing the procedure outlined in Preparation 4: RT=3.95 min m/z (ES⁺)=366.1 [M+H]⁺.

### Preparation 20: Methanesulfonic acid (R)-3-[1-(3-ethyl-[1,2,4]oxadiazol-5-yl)piperidin-4-yl]butyl ester

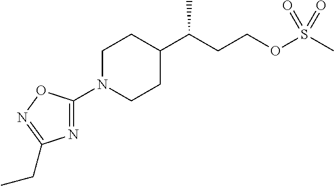

To a solution of (R)-3-[1-(3-ethyl-[1,2,4]oxadiazol-5-yl)piperidin-4-yl]butan-1-ol (2.0 g, 10.97 mmol) in DCM (30 mL) was added triethylamine (2.0 mL, 14.26 mmol) and the reaction cooled to 0° C. Methanesulfonyl chloride (1.0 L, 13.17 mmol) was added and the mixture stirred at 0° C. After complete reaction water (20 mL) was added then the organic layer was separated, washed with 1M citric acid (30 mL), sat. NaHCO₃ solution (30 mL), and dried (MgSO₄). Removal of the solvent in vacuo afforded the title compound: RT=3.29 min m/z (ES⁺)=332.2 [M+H]⁺.

### Preparation 21: 2-Chloro-5-{(R)-3-[1-(3-ethyl-[1,2,4]oxadiazol-5-yl)piperidin-4-yl]butoxy}pyrimidine

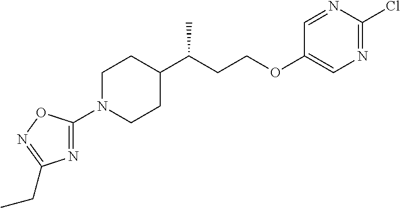

A combination of methanesulfonic acid (R)-3-[1-(3-ethyl-[1,2,4]oxadiazol-5-yl)piperidin-4-yl]butyl ester (Preparation 20, 2.22 g, 6.71 mmol), 2-chloro-5-hydroxypyrimidine (0.96 g, 7.38 mmol) and potassium carbonate (2.78 g, 20.13 mmol) in DMF (30 mL) was heated to 50° C. until the reaction was complete. The mixture was diluted with water (40 mL) and extracted into EtOAc (3×30 mL). The organic fractions were combined, washed with brine (70 mL), and dried (MgSO₄). Removal of the solvent in vacuo and purification by column chromatography (IH:EtOAc, 70:30, 60:40) afforded the title compound: RT=3.84 min; m/z (ES⁺)=366.2 [M+H]⁺.

### Preparation 22: 3-[1-(5-Chloropyrimidin-2-yl)piperidin-4-yl]propan-1-ol

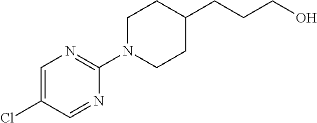

To a solution of 3-piperidin-4-yl-propan-1-ol hydrochloride (15.00 g, 0.08 mol) in DMSO (120 mL), cooled to 0° C., was added DBU (29.95 mL, 0.20 mol), dropwise, over 5 min. 2,5-dichloropyrimidine (17.43 g, 0.12 mol) was added and the mixture warmed to r.t. before heating to 110° C. for 4 h. The reaction mixture was diluted with water (200 mL) and extracted with EtOAc (3×500 mL). The organic fractions were combined, washed with 1M HCl (2×200 mL), dried (MgSO₄) and the solvent removed in vacuo. Purification by column chromatography (IH:EtOAc, 60:40) afforded the title compound: RT=3.52 min; m/z (ES⁺)=256.1 [M+H]⁺.

### Preparation 23: 5-Chloro-2-{4-[5-(2-chloropyrimidin-5-yloxy)propyl]piperidin-1-yl}pyrimidine

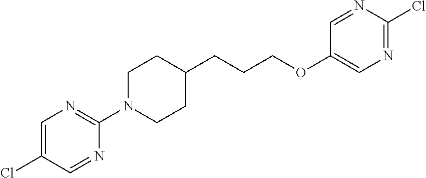

To a solution of 3-[1-(5-chloropyrimidin-2-yl)piperidin-4-yl]propan-1-ol (Preparation 22, 766 mg, 3.0 mmol) in DCM (25 mL) was added triethylamine (0.50 mL, 3.6 mmol) and the mixture was cooled to 0° C. Methanesulfonyl chloride (0.23 mL, 3.0 mmol) was added and the reaction warmed to r.t. over 15 min. 1M HCl was added and the resulting mixture poured into EtOAc. The organic layer was separated, washed with 1M HCl, brine, dried (MgSO₄), and the solvent removed in vacuo. To a solution of the material in DMF (10 mL) was added 2-chloro-5-hydroxypyrimidine (390 mg, 3.0 mmol) and potassium carbonate (828 mg, 6.0 mmol), and the mixture was heated to 80° C. for 16 h. DMF was removed in vacuo and the residue was re-dissolved in EtOAc. The organic solution was washed with brine (×2), dried (MgSO₄), and solvent removed in vacuo. Recrystalisation from the minimal volume of MeOH afforded the title compound: RT=4.53 min m/z (ES⁺)=368.1 [M+H]⁺.

### Preparation 24: 2-Chloro-5-{3-[1-(5-isopropyl-[1,2,4]oxadiazol-3-yl)piperidin-4-yl]propoxy}pyrimidine

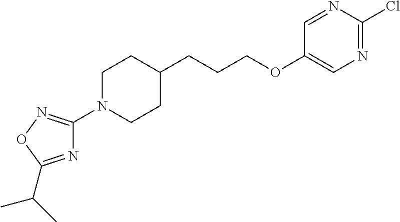

The title compound was prepared from 3-[1-(5-isopropyl-[1,2,4]oxadiazol-3-yl)piperidin-4-yl]propan-1-ol employing the procedure outlined in Preparation 23: RT=4.22 min m/z (ES⁺)=366.2 [M+H]⁺.

### Preparation 25: (3S,4S)-4-Azido-1-benzylpyrrolidin-3-ylamine

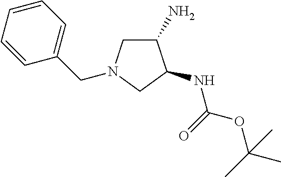

To a solution of (3S,4S)-3,4-diazido-1-benzylpyrrolidine (15.6 g, 64.10 mmol) in THF (500 mL) cooled to 0° C. was added a solution of triphenylphosphine (16.5 g, 62.81 mmol) in THF (100 mL), dropwise over 4 h and the resulting mixture was allowed to reach r.t. and stirred for 16 h. The reaction solvent was removed in vacuo and the resulting residue was re-dissolved in THF (500 mL) and water (1.3 mL) before being heated to reflux for 4 h then stirred at r.t. for 16 h. The reaction solvent was removed in vacuo and the resulting residue was triturated with Et₂O. The precipitate was filtered and the filtrate was concentrated in vacuo. The residue was taken into Et₂O again and filtered. Removal of the filtrate in vacuo followed by purification by column chromatography (IH:EtOAc, 90:10, 80:20, 50:50, 0:100 then MeOH:NH₄OH, 9:1) afforded the title compound: RT=0.77 min; m/z (ES⁺)=218.1 [M+H]⁺.

### Preparation 26: ((3S,4S)-4-Azido-1-benzylpyrrolidin-3-yl)carbamic acid tert-butyl ester

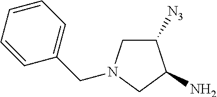

To a solution of (3S,4S)-4-azido-1-benzylpyrrolidin-3-ylamine (Preparation 25, 6.0 g, 27.74 mmol) and triethylamine (4.6 mL, 33.29 mmol) in DCM (100 mL), cooled to 0° C., was added a solution of di tert-butyldicarbonate (7.3 g, 33.29 mmol) in DCM (10 mL) dropwise over 20 min. The resulting mixture was allowed to reach r.t. and stirred for 72 h. The reaction solvent was washed with sat. NaHCO₃ solution, then brine, and dried (MgSO₄). Removal of the solvent in vacuo followed by purification by column chromatography (DCM:MeOH) afforded the title compound: ¹H NMR δ_{H} (400 MHz, CDCl₃): 7.37-7.26 (m, 5H), 4.09-4.02 (m, 1H), 3.84-3.76 (m, 1H), 3.68-3.59 (m, 2H), 3.12-3.01 (m, 1H), 2.91-2.82 (m, 1H), 2.55-2.35 (m, 2H), 1.46 (s, 9H).

### Preparation 27: ((3S,4S)-4-Amino-1-benzylpyrrolidin-3-yl)carbamic acid tert-butyl ester

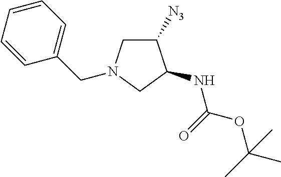

The title compound was prepared from ((3S,4S)-4-azido-1-benzylpyrrolidin-3-yl)carbamic acid tert-butyl ester (Preparation 26) employing the procedure outlined in WO2007/148185

### Preparation 28: [(3S,4S)-1-Benzyl-4-(2-oxopiperidin-1-yl)pyrrolidin-3-yl]carbamic acid tert-butyl ester

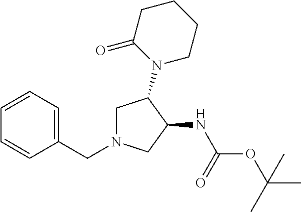

The title compound was prepared in 2 steps from ((3S,4S)-4-amino-1-benzylpyrrolidin-3-yl)carbamic acid tert-butyl ester (Preparation 27) employing the procedure outlined in WO2007/148185.

### Preparation 29: 3-Benzyloxy-2-methylpropionaldehyde

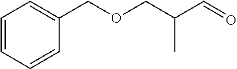

To a solution of 3-benzyloxy-2-methylpropan-1-ol (1.91 g, 10.60 mmol) in DCM (30 mL) under argon, cooled to 0° C., was added dess-martin periodinane (4.94 g, 11.66 mmol) and the mixture was stirred for 4 h. The reaction was diluted with EtOAc (150 mL) and washed with sat. NaHCO₃ solution (2×100 mL), water (100 mL), brine (100 mL), and dried (MgSO₄). Removal of the solvent in vacuo followed by purification by column chromatography (IH:EtOAc, 80:20) afforded the title compound: ¹H NMR δ_{H} (400 MHz, CDCl₃): 9.74 (d, 1H), 7.41-7.27 (m, 5H), 4.54 (s, 2H), 3.74-3.62 (m, 2H), 2.73-2.63 (m, 1H), 1.15 (d, 3H).

### Preparation 30: 5-Benzyloxy-4-methylpent-2-enoic acid methyl ester

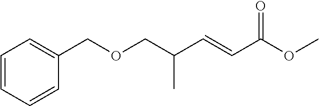

To a dry solution of (methoxycarbonylmethylene)triphenylphosphorane (2.76 g, 8.25 mmol) in DMF (5 mL) under argon was added a solution of 3-benzyloxy-2-methylpropionaldehyde (Preparation 29, 1.4 g, 7.86 mmol) in DMF (9 mL) and the mixture was stirred at r.t. for 70 h. The reaction was diluted with water (100 mL) and extracted with EtOAc (3×40 mL). The organic fractions were combined, washed with water (60 mL), sat. NaHCO₃ solution (60 mL), brine (60 mL) and dried (MgSO₄). Removal of the solvent in vacuo followed by purification by column chromatography (IH:EtOAc, 85:15) afforded the title compound: RT=3.59 min; m/z (ES⁺)=235.1 [M+H]⁺.

### Preparation 31: 5-Hydroxy-4-methyl pentanoic acid methyl ester

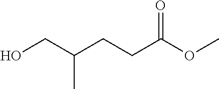

To a solution of 5-benzyloxy-4-methylpent-2-enoic acid methyl ester (Preparation 30, 600 mg, 2.56 mmol), in EtOH (30 mL) was added palladium on carbon (10%, 409 mg, 0.36 mmol) and the reaction was stirred under an atmosphere of hydrogen for 16 h. The mixture was filtered through celite, washing with EtOH (60 mL) then the filtrate was concentrated in vacuo to afford the title compound: ¹H NMR δ_{H} (400 MHz, DMSO-d₆): 4.45-4.38 (m, 1H), 3.58 (s, 3H), 3.26-3.17 (m, 2H), 2.38-2.23 (m, 2H), 1.69-1.59 (m, 1H), 1.52-1.42 (m, 1H), 1.36-1.24 (m, 1H), 0.81 (d, 3H).

### Preparation 32: 4-Methyl-5-oxopentanoic acid methyl ester

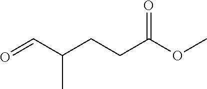

A solution of 5-hydroxy-4-methyl pentanoic acid methyl ester (Preparation 31, 370 mg, 2.53 mmol) in DCM (8 mL) under argon was cooled to 0° C. The solution was treated with dess-martin periodinane (1.18 g, 2.78 mmol) and then stirred at 0° C. for 3 h. The mixture was diluted with DCM (50 mL) and washed with water (50 mL). The water layer was extracted with DCM (50 mL) and the organic layers were combined, washed with brine (50 mL) and dried (MgSO₄). Removal of the solvent in vacuo followed by purification by column chromatography (DCM) afforded the title compound: ¹H NMR δ_{H} (400 MHz, CDCl₃): 9.64 (d, 1H), 3.68 (s, 3H), 2.48-2.34 (m, 3H), 2.13-2.00 (m, 1H), 1.77-1.65 (m, 1H), 1.14 (d, 3H).

### Preparation 33: [(3S,4S)-1-Benzyl-4-(5-methyl-2-oxopiperidin-1-yl)pyrrolidin-3-yl]carbamic acid tert-butyl ester

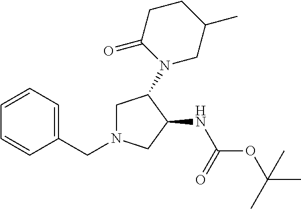

To a solution of 4-methyl-5-oxopentanoic acid methyl ester (Preparation 32, 230 mg, 1.60 mmol) in DCM (8 mL) under argon was added ((3S,4S)-4-amino-1-benzylpyrrolidin-3-yl)carbamic acid tert-butyl ester (Preparation 27, 465 mg, 1.60 mmol) and the reaction was stirred at r.t. for 4 h. The reaction was then treated with sodium triacetoxyborohydride (406 mg, 1.91 mmol) and stirring continued at r.t. for 72 h. The reaction was quenched with sat. Na₂CO₃ solution (20 mL), and then extracted with EtOAc (40 mL). The organic portion was washed with brine, dried (MgSO₄) and solvent was removed in vacuo. The resulting residue was dissolved in toluene (6 mL) and heated to 90° C. for 4 h. After cooling, the crude reaction mixture was concentrated onto silica gel and purified by column chromatography (EtOAc:IH:Et₃N, 9:1:0.1) to afford the title compound: RT=2.47 min; m/z (ES⁺)=388.2 [M+H]⁺.

### Preparation 34: 5-Hydroxy-3-methylpentanoic acid methyl ester

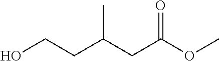

A solution of monomethyl-3-methylglutarate (1.0 g, 6.24 mmol) in THF (15 mL), under argon, was cooled to 0° C. Boron-dimethylsilfide complex (1M in DCM, 9.37 mL, 9.37 mmol) was added, dropwise, over 20 min and the reaction was stirred for 2 h before warming to r.t., and stirring for a further 20 h. The reaction was quenched by addition of a mixture of water and AcOH (1:1, 2 mL) and the mixture was extracted with EtOAc (100 mL). The organic phase was washed with cold sat. NaHCO₃ solution (3×50 mL), brine (50 mL) and dried (MgSO₄). Removal of the solvent in vacuo afforded the title compound: ¹H NMR δ_{H} (400 MHz, CDCl₃): 3.73-3.64 (m, 5H), 2.39-2.31 (m, 1H), 2.25-2.11 (m, 2H), 1.64-1.48 (m, 3H), 1.00-0.96 (m, 3H).

### Preparation 35: 3-Methyl-5-oxopentanoic acid methyl ester

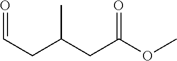

To a dry solution of 5-hydroxy-3-methylpentanoic acid methyl ester (Preparation 34, 800 mg, 5.47 mmol) in DCM (15 mL), under argon, cooled to 0° C., was added dess-martin periodinane (2553 mg, 6.02 mmol) and the resulting mixture was stirred fo 4 h, allowed to reach r.t., and stirred for a further 16 h. The mixture was diluted with DCM (50 mL), then washed with sat. NaHCO₃ solution (2×50 mL), brine (50 mL), and dried (MgSO₄). Removal of the solvent in vacuo afforded the title compound: ¹H NMR δ_{H} (400 MHz, CDCl₃): 9.76 (s, 1H), 3.67 (s, 3H), 2.60-2.49 (m, 2H), 2.42-2.27 (m, 3H), 1.05-1.01 (m, 3H).

### Preparation 36: [(3S,4S)-1-Benzyl-4-(4-methyl-2-oxopiperidin-1-yl)pyrrolidin-3-yl]carbamic acid tert-butyl ester

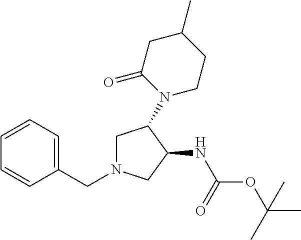

To a dry solution of ((3S,4S)-4-amino-1-benzylpyrrolidin-3-yl)carbamic acid tert-butyl ester (Preparation 27, 600 mg, 2.06 mmol) in DCM (10 mL) under argon was added 3-methyl-5-oxopentanoic acid methyl ester (Preparation 35, 312 mg, 2.16 mmol) and the reaction was stirred at r.t. for 5 h. Sodium triacetoxyborohydride (524 mg, 2.47 mmol) was added and the reaction stirred for a further 18 h. The mixture was diluted with DCM (50 mL) and washed with sat. Na₂CO₃ solution (50 mL). The aqueous phase was extracted with DCM (30 mL) then organic phases combined, washed with sat. Na₂CO₃ solution (50 mL), brine (50 mL) and dried (MgSO₄). Removal of the solvent in vacuo and purification by column chromatography (IH:EtOAc, 50:50, 30:70) afforded the title compound: RT=2.42 min; m/z (ES⁺)=388.2 [M+H]⁺.

### Preparation 37: [(3S,4S)-1-Benzyl-4-(4-bromobutyrylamino)pyrrolidin-3-yl]carbamic acid tert-butyl ester

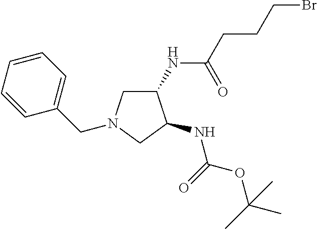

To a solution of ((3S,4S)-4-amino-1-benzylpyrrolidin-3-yl)carbamic acid tert-butyl ester (Preparation 27, 302 mg, 1.04 mmol) in DCM (15 mL) under argon was added triethylamine (159 μL, 1.14 mmol) and the reaction was cooled to 0° C. 4-Bromobutyryl chloride (126 μL, 1.09 mmol) was added, dropwise, to the solution and the resulting mixture was allowed to stir for 2 h. The reaction mixture was partitioned with sat. Na₂CO₃ solution and the organic layer removed. The aqueous phase was extracted with DCM, then the organic fractions combined, passed through a phase separator, and the solvent removed in vacuo to afford the title compound: RT=2.51 min; m/z (ES⁺)=440.2, 442.2 [M+H]⁺.

### Preparation 38: ((3′S,4′S)-1′-Benzyl-2-oxo-[1,3]bipyrrolidinyl-4′-yl)carbamic acid tert-butyl ester

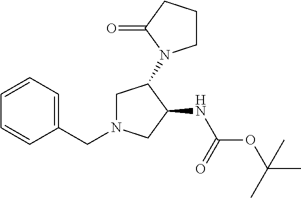

To a solution of [(3S,4S)-1-benzyl-4-(4-bromobutyrylamino)pyrrolidin-3-yl]carbamic acid tert-butyl ester (Preparation 37, 532 mg, 1.21 mmol) in a mixture of THF (10 mL) and DMF (5 mL), cooled to 0° C., was added sodium hydride (60% in mineral oil, 101 mg, 2.54 mmol) and the reaction was stirred for 10 min, then allowed to reach r.t. and stirred for 72 h. The reaction mixture was concentrated in vacuo and the resulting residue dissolved in EtOAc, washed with water (×3), dried (MgSO₄) and solvent removed in vacuo. Purification by column chromatography (DCM:MeOH, 100:0, 98:2) afforded the title compound: RT=1.99 min; m/z (ES⁺)=360.2 [M+H]⁺.

### Preparation 39: [(3S,4S)-1-Benzyl-4-(5-bromo-4,4-difluoropentanoylamino)pyrrolidin-3-yl]carbamic acid tert-butyl ester

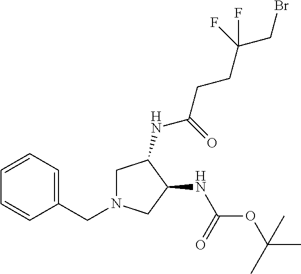

A combination of 5-bromo-4,4-difluoropentanoic acid (1.34 g, 6.17 mmol), EDCI (1.89 g, 9.88 mmol), HOBt (0.83 g, 6.17 mmol) and triethylamine (2.58 mL, 18.52 mmol) in DMF (15 mL) was stirred at r.t. for 20 min. To the reaction was added a solution of ((3S,4S)-4-amino-1-benzylpyrrolidin-3-yl)carbamic acid tert-butyl ester (Preparation 27, 1.89 g, 6.48 mmol) in DMF (10 mL) and the reaction was heated to 50° C. for 16 h. After this time further portions of EDCI (0.59 g, 3.09 mmol) and triethylamine (0.43 mL, 3.09 mmol) were added and heating continued at 40° C. for 24 h. DMF was removed in vacuo and the crude residue was taken into water. The mixture was extracted with EtOAc (3×100 mL) then the organic fractions were combined, washed with brine:water (1:1) and dried (MgSO₄). Removal of the solvent in vacuo and purification by column chromatography (DCM:MeOH:NH₄OH, 99:1:0.1, 96:4:0.1) afforded the title compound: RT=2.92 min; m/z (ES⁺)=490.1, 492.1 [M+H]⁺.

### Preparation 40: [(3S,4S)-1-Benzyl-4-(5,5-difluoro-2-oxopiperidin-1-yl)pyrrolidin-3-yl]carbamic acid tert-butyl ester

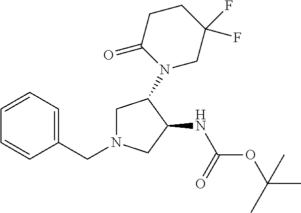

A solution of [(3S,4S)-1-benzyl-4-(5-bromo-4,4-difluoropentanoylamino)pyrrolidin-3-yl]carbamic acid tert-butyl ester (Preparation 39, 600 mg, 1.22 mmol) in DMF (6 mL) was cooled to 0° C. Sodium hydride (60% mineral oil, 98 mg, 2.44 mmol) was added and the reaction allowed to stir, gradually warming to r.t. over 30 min. Sat. NH₄Cl solution was added dropwise, until bubbling ceased, and the solvent was removed in vacuo. Purification by column chromatography (DCM:MeOH:NH₄OH, 99:1:0.1, 98:2:0.2) afforded the title compound: RT=2.42 min; m/z (ES⁺)=410.2 [M+H]⁺.

### Preparation 41: [(3S,4S)-4-(2-Oxopiperidin-1-yl)pyrrolidin-3-yl]carbamic acid tert-butyl ester

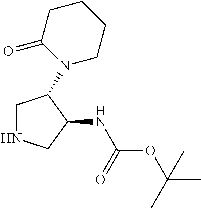

A solution of [(3S,4S)-1-benzyl-4-(2-oxopiperidin-1-yl)pyrrolidin-3-yl]carbamic acid tert-butyl ester (Preparation 28, 2.6 g, 7.07 mmol) in MeOH (140 mL) was passed through an H-Cube apparatus (10% pd/C Catcart 70, 10 bar, 90° C.) at a flow rate of 1 mL per min. The solvent was removed in vacuo to afford the title compound: ¹H NMR δ_{H} (400 MHz, CDCl₃): 5.25-5.07 (m, 1H), 4.85-4.62 (m, 1H), 4.34-4.07 (m, 1H), 3.49-3.28 (m, 3H), 3.24 (s, 1H), 2.99 (s, 1H), 2.87-2.73 (m, 1H), 2.52-2.39 (m, 2H), 2.38-2.22 (m, 2H), 1.91-1.74 (m, 1H), 1.54-1.38 (m, 9H).

The following compounds were prepared from the appropriate benzyl protected amine employing a procedure similar to that outlined in Preparation 41:

| Prep | | | |
|---|---|---|---|
| No. | Structure | Name | LCMS |
| 42 | 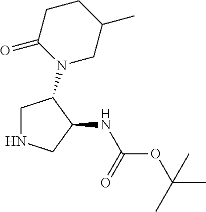 | [(3S,4S)-4-(5- Methyl- 2-oxopiperidin-1- yl)pyrrolidin-3- yl]carbamic acid tert-butyl ester | RT = 2.17 min; m/z (ES⁺) = 298.2 [M + H]⁺. |
| 43 | 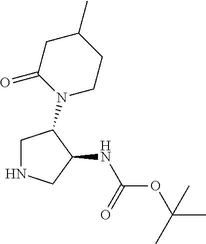 | [(3S,4S)-4-(4- Methyl- 2-oxopiperidin-1- yl)pyrrolidin-3- yl]carbamic acid tert-butyl ester | RT = 2.07 min; m/z (ES⁺) = 298.2 [M + H]⁺ |
| 44 | 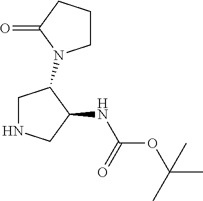 | (3′S,4′S)-2-Oxo- [1,3′]bipyrroli- dinyl-4-yl) carbamic acid tert-butyl ester | RT = 1.60 min; m/z (ES⁺) = 270.1 [M + H]⁺ |
| 45 | 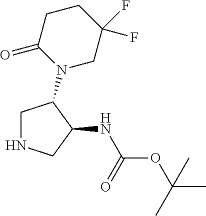 | [(3S,4S)-4-(5,5- Difluoro-2- oxopiperidin-1- yl)pyrrolidin-3- yl]carbamic acid tert-butyl ester | RT = 2.04 min; m/z (ES⁺) = 320.2 [M + H]⁺. |

### Preparation 46: 3-(2,5-Difluorophenyl)-4-nitrobutyric acid methyl ester

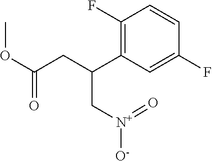

To a solution of (2E)-3-(2,5-difluorophenyl)acrylic acid (21.10 g, 114.7 mmol) in a mixture of DCM and MeOH (DCM:MeOH, 4:1, 250 mL) was added a solution of trimethylsilyldiazomethane (2M in Et₂O, 57.34 mL, 114.7 mmol) over 15 min and the resulting mixture was stirred at r.t. until complete. AcOH was added, dropwise, until the reaction turned colourless, and solvent was removed in vacuo. The residue was re-dissolved in MeCN (114 mL) and nitromethane (7.45 mL, 137.6 mmol) was added. The mixture was cooled to 0° C. and DBU (17.49 mL, 117.0 mmol) was added, dropwise, over 30 min. The reaction was allowed to reach r.t. before stirring for 16 h. Removal of the solvent in vacuo and purification by column chromatography (IH:EtOAc, 95:5, 90:10) afforded the title compound: ¹H NMR δ_{H} (400 MHz, CDCl₃): 7.18-7.00 (m, 3H), 4.91-4.77 (m, 2H), 4.27-4.17 (m, 1H), 3.75 (s, 3H), 2.91 (m, 2H).

### Preparation 47: (trans)-1-Benzyl-4-(2,5-difluoronhenyl)-5-nitropiperidin-2-one

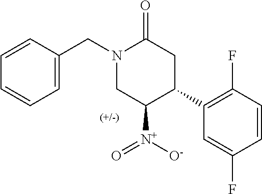

A combination of 3-(2,5-difluorophenyl)-4-nitrobutyric acid methyl ester (Preparation 46, 16.27 g, 62.81 mmol), paraformaldehyde (1.94 g, 64.63 mmol) and benzylamine (13.7 mL, 125.62 mmol) in EtOH was heated to 90° C. in a sealed tube for 16 h. After complete reaction the mixture was partitioned between EtOAc (400 mL) and 2M HCl (600 mL). The organic fraction was separated, washed with brine, dried (MgSO₄), and solvent removed in vacuo. Purification by column chromatography (IH:EtOAc, 70:30) afforded the title compound: RT=3.72 min m/z (ES⁺)=347.1 [M+H]⁺.

### Preparation 48: (trans)-1-Benzyl-4-(2,5-difluorophenyl)-3-nitropiperidine hydrochloride

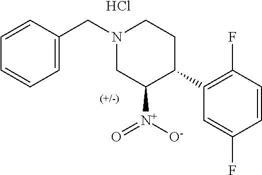

To a solution of (trans)-1-benzyl-4-(2,5-difluorophenyl)-5-nitropiperidin-2-one (Preparation 47, 10.44 g, 30.17 mmol) in THF (90 mL) under argon was added borane dimethylsulfide complex (2.0M in DCM, 45.3 mL, 90.60 mmol) and the reaction was heated to 70° C. for 3 h. After cooling to r.t. the mixture was diluted with MeOH (20 mL) and 1M HCl (30 mL) was added. The mixture was stirred for 10 min before removal of the solvent in vacuo. Further portions of MeOH (20 mL) and 1M HCl (20 mL) were added and the reaction stirred for 10 min. Removal of the solvent in vacuo afforded the title compound: RT=3.30 min m/z (ES⁺)=333.1 [M+H]⁺.

### Preparation 49: [(3R,4R)-1-Benzyl-4-(2,5-difluorophenyl)piperidin-3-yl]carbamic acid tert-butyl ester

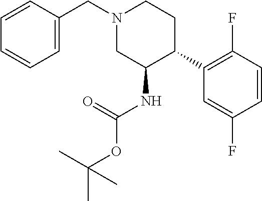

A combination of (trans)-1-benzyl-4-(2,5-difluorophenyl)-3-nitropiperidine hydrochloride (Preparation 48, 11.12 g, 30.17 mmol) and zinc dust (15.69 g, 241.36 mmol) in a mixture of AcOH and EtOH (1:1, 110 mL) was heated to 80° C. After complete reaction the mixture was filtered and the solvent removed in vacuo. To a solution of the resulting residue in MeOH (30 mL) was added HCl in dioxane (4M, 30 mL), and the solvent removed in vacuo. The material was triturated with Et₂O (×2) and toluene (×3) to afford the amine as the hydrochloride salt. To a solution of the product in a mixture of THF (150 mL) and water (75 mL), cooled to 0° C., was added triethylamine (12.6 mL, 90.51 mmol), followed by di-tert-butyl dicarbonate (9.59 g, 45.26 mmol). The mixture was allowed to reach r.t. and stirred for 16 h, until complete. The mixture was partitioned between EtOAc (750 mL) and water (200 mL) and the organic phase separated. The aqueous phase was extracted with EtOAc (500 mL) then organic fractions were combined, dried (MgSO₄) and the solvent removed in vacuo. Purification by column chromatography (IH:EtOAc, 80:20) followed by further purification by chiral HPLC (IH:IPA:DEA, 90:10:0.1, 15 ml/min, 270 nm, RT=9.8 min) afforded the title compound: RT=2.68 min m/z (ES⁺)=403.2 [M+H]⁺.

### Preparation 50: [(3R,4R)-4-(2,5-Difluorophenyl)piperidin-3-yl]carbamic acid tert-butyl ester

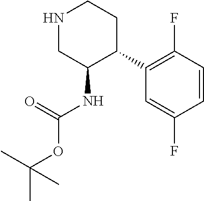

A solution of [(3R,4R)-1-benzyl-4-(2,5-difluorophenyl)piperidin-3-yl]carbamic acid tert-butyl ester (Preparation 49, 1.89 g, 4.70 mmol) in MeOH (94 mL) was passed through an H-Cube apparatus (10% pd/C Catcart 70, 30 bar, 80° C.) at a flow rate of 1 mL per min. The solvent was removed in vacuo to afford the title compound: RT=2.37 min; m/z (ES⁺)=313.2[M+H]⁺.

### Preparation 51: 3-(2-Fluorophenyl)-4-nitrobutyric acid methyl ester

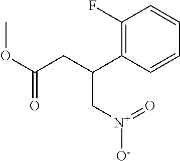

To a solution of methyl (E)-3-(2-fluorophenyl)-2-propenoate (19.4 g, 108 mmol) in MeCN (100 mL) under an atmosphere of argon, cooled to 0° C., was added nitromethane (7 mL, 130 mmol) and DBU (16 mL, 107 mmol). The ice bath was removed and the reaction stirred at r.t. for 16 h. The mixture was partitioned between Et₂O (400 mL) and 1M HCl (400 mL), and the organic phase was separated. The aqueous phase was extracted further with Et₂O (2×200 mL) then the organic fractions were combined, dried (MgSO₄) and concentrated in vacuo. Purification by column chromatography (IH:EtOAc, 3:1, 2.5:1) afforded the title compound: ¹H NMR δ_{H} (400 MHz , CDCl₃): 7.33-7.20 (m, 2H), 7.15-7.03 (m, 2H), 4.84-4.73 (m, 2H), 4.24-4.13 (m, 1H), 3.65 (s, 3H), 2.88-2.82 (m, 2H).

### Preparation 52: (trans)-1-Benzyl-4-(2-fluorophenyl)-5-nitropiperidin-2-one

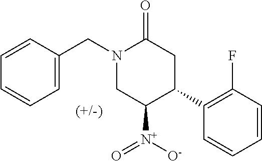

A combination of 3-(2-fluorophenyl)-4-nitrobutyric acid methyl ester (Preparation 51, 10.50 g, 43.5 mmol), paraformaldehyde (1.39 g, 46.3 mmol) and benzylamine (9.67 mL, 87.1 mmol) in EtOH (100 mL) was heated to 100° C. in a sealed tube for 16 h. After complete reaction the solvent was concentrated in vacuo and the resulting residue partitioned between EtOAc (700 mL) and 2M HCl (300 mL). The organic fraction was separated, washed with brine, dried (MgSO₄), and the solvent removed in vacuo. Purification by column chromatography (IH:EtOAc, 1.5:1, 1:1) afforded the title compound: ¹H NMR δ_{H} (400 MHz , CDCl₃): 7.40-7.01 (m, 9H), 5.09-5.02 (m, 1H), 4.87-4.80 (m, 1H), 4.54-4.46 (m, 1H), 4.14-4.06 (m, 1H), 3.88-3.80 (m, 1H), 3.63-3.54 (m, 1H), 2.94-2.79 (m, 2H)

### Preparation 53: (trans)-1-Benzyl-4-(2-fluorophenyl)-3-nitropiperidine hydrochloride

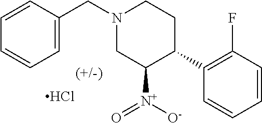

To a solution of (trans)-1-benzyl-4-(2-fluorophenyl)-5-nitropiperidin-2-one (Preparation 52, 8.0 g, 24.4 mmol) in THF (100 mL), under argon, was added borane dimethylsulfide complex (2.0M in THF, 37 mL, 74.0 mmol) and the reaction was heated to 70° C. for 5 h before allowing to stir at r.t. for 16 h. To the mixture was cautiously added MeOH (50 mL), followed by 1M HCl (20 mL), and the resulting mixture was stirred for 20 min before removal of the solvent in vacuo. Further portions of MeOH (100 mL) and 1M HCl (100 mL) were added and the solvent removed in vacuo to afford the title compound: RT=3.07 min m/z (ES⁺)=315.1 [M+H]⁺.

### Preparation 54: [(3R,4R)-1-Benzyl-4-(2-fluorophenyl)piperidin-3-yl]carbamic acid tert-butyl ester

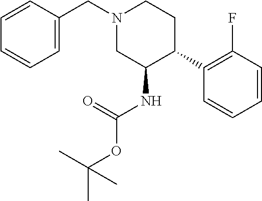

A combination of (trans)-1-benzyl-4-(2-fluorophenyl)-3-nitropiperidine hydrochloride (Preparation 53, 6.9 g, 19.7 mmol) and zinc dust (10.4 g, 159.1 mmol) in a mixture of AcOH and EtOH (1:1, 150 mL) was heated to 75° C. After complete reaction the mixture was diluted with MeOH (100 mL), filtered, and the solvent removed in vacuo. To a solution of the resulting residue in THF (300 mL) was added triethylamine (20 mL) followed by di-tert-butyl dicarbonate (6.2 g, 28.4 mmol), and the reaction was stirred at r.t. for 2 h. The solvent was concentrated in vacuo, and the resulting residue re-dissolved in EtOAc (500 mL). The solution was washed with 1M NaOH solution, brine, dried (MgSO₄), and the solvent removed in vacuo. Recrystallisation from IH:EtOAc (20:1) followed by further purification by chiral HPLC (IH:IPA:DEA, 92:8:0.1, 15 ml/min, 265 nm, RT=9.0 min) afforded the title compound: RT=2.72 min m/z (ES⁺)=385.2 [M+H]⁺.

### Preparation 55: [(3R,4R)-4-(2-Fluorophenyl)piperidin-3-yl]carbamic acid tert-butyl ester

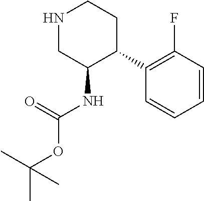

The title compound was prepared from [(3R,4R)-1-benzyl-4-(2-fluorophenyl)piperidin-3-yl]carbamic acid tert-butyl ester (Preparation 54) employing the procedure outlined in Preparation 41, but using the following conditions: 10% pd/C Catcart 30, 30 bar, 90° C., 1 mL per min, 13 mM concentration in MeOH: RT=2.19 min m/z (ES⁺)=295.2 [M+H]⁺.

### Preparation 56: 2,4-Difluoro-1-((E)-2-nitrovinyl)benzene

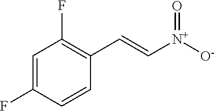

To a solution of 2,4-difluorobenzaldehyde (25.0 g, 0.18 mol) and nitromethane (11.4 mL, 0.21 mol) in MeOH (53 mL) under argon, cooled to −15° C., was added a solution of NaOH (7.4 g, 0.19 mol) in water (26 mL), dropwise over 20 min. The resulting mixture was stirred at −15° C. and a precipitate formed after 30 min. More MeOH was added to form a slurry and stirring continued for 15 min before allowing the reaction to warm to 0° C. Ice water was added and the mixture stirred for 15 min before adding 4M HCl (100 mL). The organic fraction was extracted into DCM (3×300 mL), dried (Na₂SO₄) and the solvent removed in vacuo. The residue (10.00 g, 50 mmol) was dissolved in acetic anhydride (8.13 mL, 90 mol) and cooled to 0° C. under argon. DMAP (0.42 g, 3 mmol) was added and the reaction stirred at this temperature for 20 min before warming the mixture to to r.t. and allowing it to stir for a further 16 h. The reaction solvent was removed in vacuo and the resulting residue re-dissolved in DCM. Any remaining acetic anhydride was destroyed by the addition of a small volume of 1M NaOH solution, then the resulting solution was dried (MgSO₄) and concentrated in vacuo. Purification by column chromatography (DCM) afforded the title compound: RT=3.60 min; m/z (ES⁺)=186.1 [M+H]⁺.

### Preparation 57: (trans)-1-Benzyl-3-(2,4-difluorophenyl)-4-nitropyrrolidine

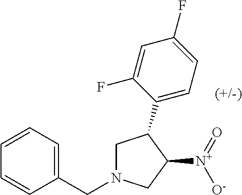

A solution of 2,4-difluoro-1-((E)-2-nitrovinyl)benzene (Preparation 56, 8.0 g, 43.0 mmol) in DCM (250 mL) under argon, was cooled to −30° C., and N-(methoxymethyl)-N-(trimethylsilylmethyl)benzylamine (11.7 mL, 45.0 mmol) was added so as to maintain the temperature. The reaction was stirred for 10 min before the dropwise addition of TFA (0.3 mL, 4.3 mmol), and the resulting mixture was allowed to stir at r.t. over 16 h. The reaction mixture was washed with water, then brine, and dried (Na₂SO₄). Removal of the solvent in vacuo afforded the title compound: RT=3.05 min; m/z (ES⁺)=319.1 [M+H]⁺.

### Preparation 58: [(trans)-1-Benzyl-4-(2,4-difluorophenyl)pyrrolidin-3-yl]carbamic acid tert-butyl ester

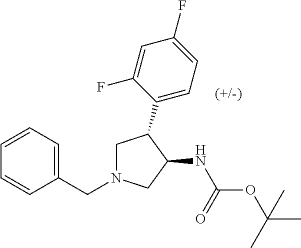

A combination of (trans)-1-benzyl-3-(2,4-difluorophenyl)-4-nitropyrrolidine (Preparation 57, 25.0 g, 0.08 mol) and zinc dust (17.8 g, 0.28 mol) in a mixture of AcOH and EtOH (1:1, 500 mL) was heated to 70° C. After 45 h a further portion of zinc dust (12.0 g, 0.18 mol) was added and heating continued for 20 min. After complete reaction the solvent was removed in vacuo. The resulting residue was re-dissolved in EtOAc, washed with sat. NaHCO₃ solution, then brine and dried (Na₂SO₄). Removal of the solvent in vacuo afforded the intermediate product (trans)-1-benzyl-4-(2,4-difluorophenyl)pyrrolidin-3-ylamine: RT=1.82 min; m/z (ES⁺)=289.1 [M+H]⁺. To a solution of the product in THF (400 mL), under argon, was added triethylamine (20.4 mL, 0.15 mol) and the solution cooled to 0° C. Di-tert-butyl dicarbonate (19.0 g, 0.09 mol) was added over 5 min, and the reaction was allowed to reach r.t. over 16 h. The solvent was removed in vacuo, then the resulting residue was re-dissolved in EtOAc, washed with brine, dried (Na₂SO₄), and the solvent removed in vacuo. To the product was added heptane (100 mL), and the suspension was sonicated until fully dissolved. The solution was allowed to stand for 60 h, allowing formation of a precipitate. The solvent was decanted and the remaining solids washed with a fresh portion of heptane (50 mL) to afford the title compound: RT=2.74 min; m/z (ES⁺)=389.3 [M+H]⁺.

### Preparation 59: [(3R,4S)-1-Benzyl-4-(2,4-difluorophenyl)pyrrolidin-3-yl]carbamic acid tert-butyl ester

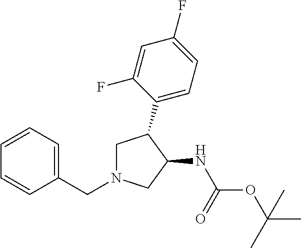

The title compound was afforded via chiral HPLC separation of [(trans)-1-benzyl-4-(2,4-difluorophenyl)pyrrolidin-3-yl]carbamic acid tert-butyl ester (Preparation 58): IH:IPA:DEA, 96:4:0.1, 15 ml/min, 270 nm, RT=9.8 min.

### Preparation 60: [(trans)-4-(2,4-Difluorophenyl)pyrrolidin-3-yl]carbamic acid tert-butyl ester

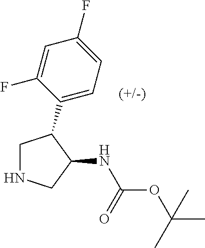

The title compound was prepared from [(trans)-1-benzyl-4-(2,4-difluorophenyl)-pyrrolidin-3-yl]carbamic acid tert-butyl ester (Preparation 58) employing the procedure outlined in Preparation 41, but at a temperature of 50° C.: RT=2.38 min; m/z (ES⁺)=299.1 [M +H]⁺.

### Preparation 61: [(3R,4S)-4-(2,4-Difluorophenyl)pyrrolidin-3-yl]carbamic acid tert-butyl ester

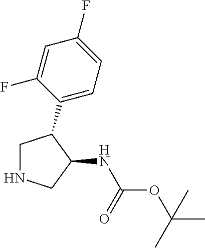

The title compound was prepared from [(3R,4S)-1-benzyl-4-(2,4-difluorophenyl)-pyrrolidin-3-yl]carbamic acid tert-butyl ester (Preparation 59) employing the procedure outlined in Preparation 41, but at a temperature of 50° C.: RT=2.38 min; m/z (ES⁺)=299.1 [M +H]⁺.

### Preparation 62: 2,4,5-Trifluoro-1-((E)-2-nitrovinyl)benzene

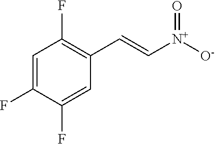

The title compound was prepared from 2,4,5-trifluorobenzaldehyde employing a similar procedure to that outlined in Preparation 56. After reaction with DMAP the crude mixture was diluted with sat. NaHCO₃ solution. The precipitate that formed was stirred for 30 min, filtered, and dried to afford the title compound: ¹H NMR δ_{H} (300 MHz , CDCl₃): 7.97-7.93 (m, 1H), 7.66-7.62 (m, 1H), 7.42-7.26 (m, 1H), 7.16-6.96 (m, 1H)

### Preparation 63: (trans)-1-Benzyl-3-(2,4,5-trifluorophenyl)-4-nitropyrrolidine

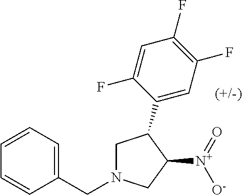

The title compound was prepared from 2,4,5-trifluoro-1-((E)-2-nitrovinyl)benzene (Preparation 62) employing the procedure outlined in Preparation 57, but the reaction was carried out at 0° C. Purification by column chromatography (Hexane:EtOAc, 100:0, 98:2, 95:5, 90:10) afforded the title compound: LCMS Method 2: RT=0.94 min; m/z (ES⁺)=337.2 [M+H]⁺.

### Preparation 64: [(trans)-1-Benzyl-4-(2,4,5-trifluorophenyl)pyrrolidin-3-yl]carbamic acid tert-butyl ester

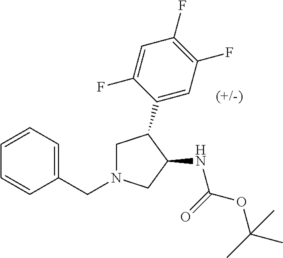

A combination of (trans)-1-benzyl-3-(2,4,5-trifluorophenyl)-4-nitropyrrolidine (Preparation 63, 11.5 g, 0.03 mol) and zinc dust (18.0 g, 0.28 mol) in a mixture of AcOH and IMS (1:1, 210 mL) was heated to 65° C. After complete reaction the mixture was filtered, washing with AcOH, and the filtrate was concentrated in vacuo. The resulting residue was re-dissolved in EtOAc, washed with sat. NaHCO₃ solution, then brine, and dried (Na₂SO₄). Removal of the solvent in vacuo and purification by column chromatography (DCM:MeOH, 100:0, 80:20) afforded the intermediate product (trans)-1-benzyl-4-(2,4,5-trifluorophenyl)-pyrrolidin-3-ylamine. LCMS Method 2: RT=0.82 min; m/z (ES⁺)=307.2 [M+H]⁺. To a solution of the product in THF (110 mL), under argon, was added triethylamine (3.9 mL, 0.04 mol) and the solution cooled to 0° C. Di-tert-butyl dicarbonate (4.7 g, 0.02 mol) was added over 5 min, then the reaction was allowed to reach r.t. and stirred for 16 h. The solvent was removed in vacuo, then the resulting residue was re-dissolved in EtOAc, washed with brine, dried (Na₂SO₄), and the solvent removed in vacuo. The product was triturated several times with heptane to afford the title compound. LCMS Method 3: RT=3.10 min; m/z (ES⁺)=407.3 [M+H]⁺.

### Preparation 65: [(trans)-4-(2,4,5-Tifluorophenyl)pyrrolidin-3-yl]carbamic acid tert-butyl ester

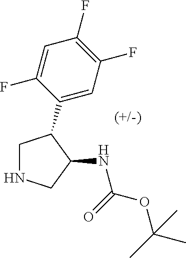

A solution of [(trans)-1-benzyl-4-(2,4,5-trifluorophenyl)pyrrolidin-3-yl]carbamic acid tert-butyl ester (Preparation 64, 40.1 g, 98.8 mmol) in a combination of IMS (325 mL) and EtOAc (50 mL) was placed in an autoclave under an atmosphere of argon. Palladium on carbon (5%, 4.0 g, 1.9 mmol) was added as a slurry in the minimum volume of toluene, then the reaction mixture was placed under an atmosphere of hydrogen (50 atm) and stirred for 72 h at r.t. The crude mixture was filtered through celite, washing with EtOAc, then the filtrate was concentrated in vacuo to afford the title compound. LCMS Method 4: RT=2.42 min; m/z (ES⁺)=317.2 [M+H]⁺.

### Preparation 66: [(3R,4S)-4-(2,4,5-Tifluorophenyl)pyrrolidin-3-yl]carbamic acid tert-butyl ester

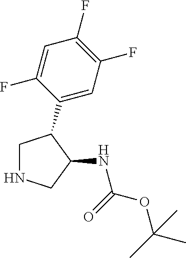

[(trans)-4-(2,4,5-Tifluorophenyl)pyrrolidin-3-yl]carbamic acid tert-butyl ester (Preparation 65, 59.5 g, 188 mmol) was suspended in EtOH (200 mL) and heated to 70° C. To the suspension was added a warm solution of (S)-(+)-naproxen (21.5 g, 93 mmol) and the mixture heated to reflux. The heat was removed and the mixture slowly allowed to cool to r.t., with stirring, for 16 h. The resulting precipitate was filtered, washing with EtOH, and partitioned between DCM (2400 mL) and 1M NaOH (600 mL). The organic phase was separated, washed with 1M NaOH, brine, then dried (MgSO₄), and the solvent removed in vacuo. The whole process was repeated for a second time to afford the title compound: ¹H NMR δ_{H} (400 MHz, CD₃OD): 7.38-7.25 (m, 1H), 7.14-7.01 (m, 1H), 4.20-4.09 (m,1H), 3.30-3.21 (m, 3H), 2.90-2.81 (m, 1H), 2.77-2.68 (m, 1H), 1.34 (br. s., 9H)

### Preparation 67: [(trans)-1-Benzyl-4-(2,5-difluorophenyl)pyrrolidin-3-yl]carbamic acid tert-butyl ester

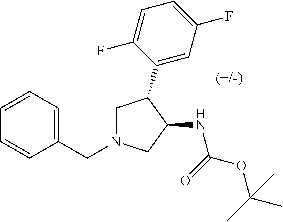

The title compound was prepared in 3 steps from 2,5-difluorobenzaldehyde employing the procedures outlined in the synthesis of [(trans)-1-benzyl-4-(2,4,5-trifluorophenyl)pyrrolidin-3-yl]carbamic acid tert-butyl ester (Preparation 64). LCMS Method 3: RT=3.04 min; m/z (ES⁺)=389.3 [M+H]⁺.

### Preparation 68: [(trans)-4-(2,5-Difluorophenyl)pyrrolidin-3-yl]carbamic acid tert-butyl ester

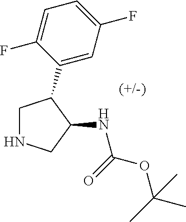

To a solution of [(trans)-1-benzyl-4-(2,5-difluorophenyl)pyrrolidin-3-yl]carbamic acid tert-butyl ester (Preparation 67, 164 g, 0.42 mol) in IMS (5.75 L) was added palladium on carbon (10%, 50% wet, 16.4 g) and the reaction was placed under an atmosphere of hydrogen (atmospheric pressure) at 50° C. for 6 h. The mixture was filtered then the filtrate concentrated in vacuo. The residue was triturated twice with DIPE (400 mL then 100 mL) and the product dried with suction to afford the title compound: ¹H NMR δ_{H} (400 MHz, CDCl₃): 7.05-6.95 (m, 2H), 6.93-6.84 (m, 1H), 4.23-4.11 (m, 1H), 3.51-3.40 (m, 2H), 3.30-3.19 (m, 1H), 3.00-2.83 (m, 2H), 1.40 (s, 9H).

### Preparation 69: [(3R,4S)-1-Benzyl-4-(2,5-difluorophenyl)pyrrolidin-3-yl]carbamic acid tert-butyl ester

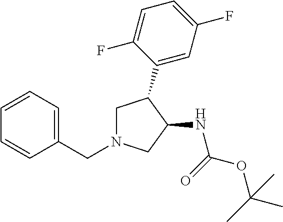

The title compound was afforded via chiral HPLC separation of [(trans)-1-benzyl-4-(2,5-difluorophenyl)pyrrolidin-3-yl]carbamic acid tert-butyl ester (Preparation 67): IH:IPA:DEA 96:4:0.1, 15 ml/min, 270 nm, RT: 10.9 min.

### Preparation 70: [(3R,4S)-4-(2,5-Difluorophenyl)pyrrolidin-3-yl]carbamic acid tert-butyl ester

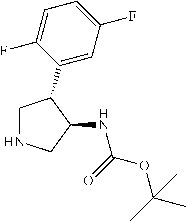

The title compound was prepared from [(3R,4S)-1-benzyl-4-(2,5-difluorophenyl)-pyrrolidin-3-yl]carbamic acid tert-butyl ester (Preparation 69) employing the procedure outlined in Preparation 41, but the reaction was carried out under 30 bar pressure. RT=2.35 min; m/z (ES⁺)=299.2 [M+H]⁺.

### Preparation 71: [(3S,4S)-1-(5-{(R)-3-[1-(3-Isopropyl-[1,2,4]oxadiazol-5-yl)piperidin-4-yl]-butoxy}-4-methylpyrimidin-2-yl)-4-(2-oxopiperidin-1-yl)pyrrolidin-3-yl]carbamic acid tert-butyl ester

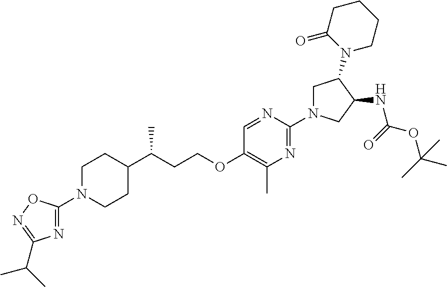

A combination of (R)-2-chloro-5-{3-[1-(3-isopropyl-[1,2,4]oxadiazol-5-yl)piperidin-4-yl]butoxy}-4-methylpyrimidine (Preparation 10, 290 mg, 0.72 mmol), [(3S,4S)-4-(2-oxopiperidin-1-yl)pyrrolidin-3-yl]carbamic acid tert-butyl ester (Preparation 41, 226 mg, 0.80 mmol) and DBU (226 mg, 1.45 mmol) in DMSO (2.5 mL) was heated in a sealed tube to 100° C. for 72 h. A further portion of [(3S,4S)-4-(2-oxopiperidin-1-yl)pyrrolidin-3-yl]carbamic acid tert-butyl ester (Preparation 41, 75 mg, 0.26 mmol) was added and heating continued for a further 48 h. The reaction mixture was diluted with EtOAc, washed with brine, and dried (Na₂SO₄). Removal of the solvent in vacuo followed by purification by column chromatography (DCM:MeOH) afforded the title compound: RT=4.12 min; m/z (ES⁺)=641.4 [M+H]⁺.

### Preparation 72: [(3S,4S)-1-(2-{(R)-3-[1-(3-isopropyl-[1,2,4]oxadiazol-5-yl)piperidin-4-yl]butoxy}pyrimidin-5-yl)-4-(2-oxopiperidin-1-yl)pyrrolidin-3-yl]carbamic acid tert-butyl ester

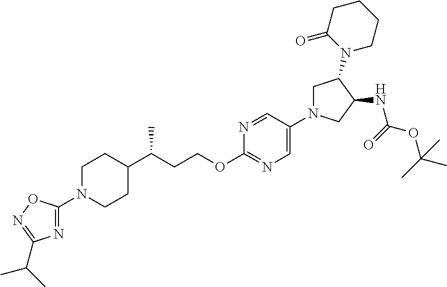

To a solution of (R)-5-bromo-2-{3-[1-(3-isopropyl-[1,2,4]oxadiazol-5-yl)piperidin-4-yl]butoxy}pyrimidine (Preparation 12, 175 mg, 0.41 mmol) in toluene (2.1 mL) was added [(3S,4S)-4-(2-oxopiperidin-1-yl)pyrrolidin-3-yl]carbamic acid tert-butyl ester (Preparation 41, 140 mg, 0.49 mmol), tris-(dibenzylideneacetone)dipalladium (19 mg, 0.02 mmol), sodium tert-butoxide (48 mg, 0.49 mmol) and (2-biphenyl)di tert-butylphosphine (25 mg, 0.08 mmol) and the mixture was heated to 75° C. for 16 h. The reaction was allowed to cool to r.t. and then partitioned between EtOAc and water. The organic phase was separated, washed with brine and dried (MgSO₄). Removal of the solvent in vacuo followed by purification by column chromatography (DCM:MeOH, 94:6) afforded the title compound: RT=3.90 min; m/z (ES⁺)=627.4 [M+H]⁺.

### Preparation 73: [(3S,4S)-1-(5-{(R)-3-[1-(3-Isopropyl-[1,2,4]oxadiazol-5-yl)piperidin-4-yl]butoxy}pyrazin-2-yl)-4-(2-oxopiperidin-1-yl)pyrrolidin-3-yl]carbamic acid tert-butyl ester

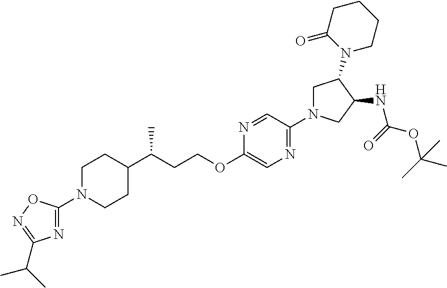

A solution of (R)-2-bromo-5-{3-[1-(3-isopropyl-[1,2,4]oxadiazol-5-yl)piperidin-4-yl]butoxy}pyrazine (Preparation 13, 140 mg, 0.33 mmol) in dioxane (4.5 mL) was added to a mixture of [(3S,4S)-4-(2-oxopiperidin-1-yl)pyrrolidin-3-yl]carbamic acid tert-butyl ester (Preparation 41, 112 mg, 0.4 mmol) and potassium tert-butoxide (111 mg, 1.15 mmol) in a microwave tube under argon. tris-(Dibenzylideneacetone)dipalladium (30 mg, 0.03 mmol) and 2,8,9-triisobutyl-2,5,8,9-tetraaza-1-phosphabicyclo-[3.3.3]undecane (12 μL, 0.03 mmol) were added and the mixture was bubbled with argon for 15 min then heated in a microwave reactor at 120° C. for 30 min. The mixture was diluted with EtOAc, then washed with water, brine, and dried (MgSO₄). Removal of the solvent in vacuo followed by purification by column chromatography (DCM:MeOH, 97:3, 96:4) afforded the title compound: RT=4.32 min; m/z (ES⁺)=627.4 [M+H]⁺.

### Preparation 74: [(3S,4S)-1-(5-{(R)-3-[1-(3-Isopropyl-[1,2,4]oxadiazol-5-yl)piperidin-4-yl]butoxy}pyrimidin-2-yl)-4-(5-methyl-2-oxopiperidin-1-yl)pyrrolidin-3-yl]carbamic acid tert-butyl ester

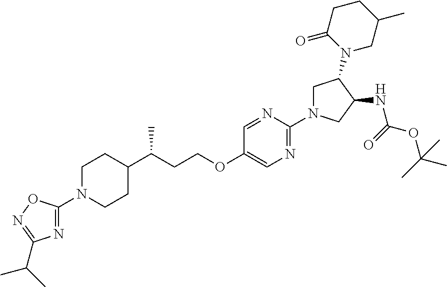

To a solution of (R)-2-chloro-5-{3-[1-(3-isopropyl-[1,2,4]oxadiazol-5-yl)piperidin-4-yl]butoxy}pyrimidine (Preparation 7, 140 mg, 0.37 mmol) and [(3S,4S)-4-(5-methyl-2-oxopiperidin-1-yl)pyrrolidin-3-yl]carbamic acid tert-butyl ester (Preparation 42, 132 mg, 0.44 mmol) in DMSO (5 mL) under argon was added DBU (138 μL, 0.92 mmol) and the reaction was heated to 115° C. for 16 h. The mixture was partitioned between water (100 mL) and EtOAc (100 mL) and the water layer was separated and extracted with a further portion of EtOAc (50 mL). The organic layers were combined, washed with water (100 mL), brine (100 mL) and dried (MgSO₄). Removal of the solvent in vacuo followed by purification by column chromatography (DCM:MeOH:NH₄OH, 98:2:0.2) afforded the title compound: RT=4.26 min; m/z (ES⁺)=641.4 [M+H]⁺.

### Preparation 75: [(3S,4S)-1-(5-{(R)-3-[1-(3-Isopropyl-[1,2,4]oxadiazol-5-yl)piperidin-4-yl]butoxy}pyrimidin-2-yl)-4-((R)-5-methyl-2-oxopiperidin-1-yl)pyrrolidin-3-yl]carbamic acid tert-butyl ester

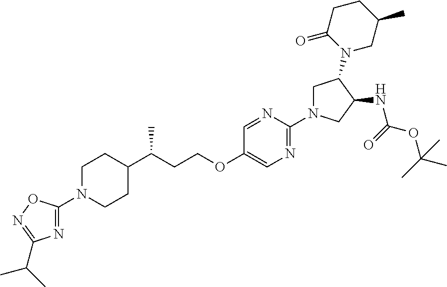

The title compound was afforded via chiral HPLC separation of [(3S,4S)-1-(5-{(R)-3-[1-(3-isopropyl-[1,2,4]oxadiazol-5-yl)piperidin-4-yl]butoxy}pyrimidin-2-yl)-4-(5-methyl-2-oxopiperidin-1-yl)pyrrolidin-3-yl]carbamic acid tert-butyl ester (Preparation 74): Daicel chiral pack IA 250×20 mm, MeOH:THF, 80:20, 15 mL/min, 250 nm.

### Preparation 76: [(3S,4S)-1-(5-{(R)-3-[1-(3-Isopropyl-[1,2,4]oxadiazol-5-yl)piperidin-4-yl]butoxy}pyrimidin-2-yl)-4-((S)-5-methyl-2-oxopiperidin-1-yl)pyrrolidin-3-yl]carbamic acid tert-butyl ester

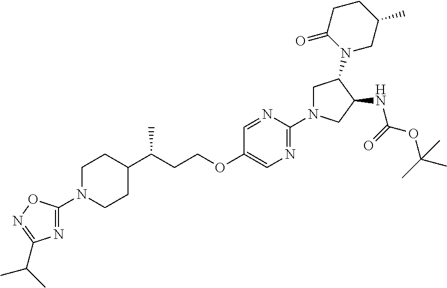

The title compound was afforded via chiral HPLC separation of [(3S,4S)-1-(5-{(R)-3-[1-(3-isopropyl-[1,2,4]oxadiazol-5-yl)piperidin-4-yl]butoxy}pyrimidin-2-yl)-4-(5-methyl-2-oxopiperidin-1-yl)pyrrolidin-3-yl]carbamic acid tert-butyl ester (Preparation 74): Daicel chiral pack IA 250×20 mm, MeOH:THF, 80:20, 15 mL/min, 250 nm.

### Preparation 77: [(trans)-4-(2,5-Difluorophenyl)-1-(5-{(R)-3-[1-(3-isopropyl-[1,2,4]oxadiazol-5-yl)piperidin-4-yl]butoxy}pyrimidin-2-yl)pyrrolidin-3-yl]carbamic acid tert-butyl ester

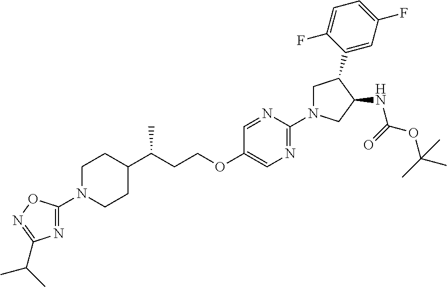

To a solution of (R)-2-chloro-5-{3-[1-(3-isopropyl-[1,2,4]oxadiazol-5-yl)piperidin-4-yl]butoxy}pyrimidine (Preparation 7, 200 mg, 0.53 mmol) and [(trans)-4-(2,5-difluorophenyl)pyrrolidin-3-yl]carbamic acid tert-butyl ester (Preparation 68, 314 mg, 1.05 mmol) in DMSO (2.5 mL), under argon, was added DBU (79 μL, 0.53 mmol) and the mixture was heated to 100° C. in a sealed tube until no further reaction was observed. The mixture was cooled to r.t. and partitioned between EtOAc and brine. The organic phase was separated, dried (Na₂SO₄) and solvent removed in vacuo. Purification by column chromatography (DCM:MeOH) afforded the title compound: RT=4.82 min; m/z (ES⁺)=642.3 [M+H]⁺.

### Preparation 78: 4-((R)-3-{2-[(3S,4S)-3-tert-Butoxycarbonylamino-4-(2-oxopiperidin-1-yl)pyrrolidin-1-yl]pyrimidin-5-yloxy}-1-methylpropyl)piperidine-1-carboxylic acid isopropyl ester

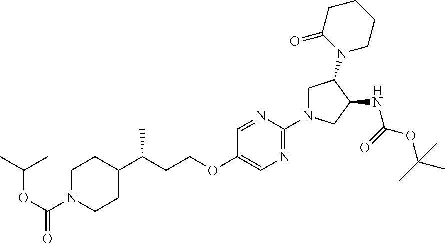

To a solution of 4-[(R)-3-(2-chloropyrimidin-5-yloxy)-1-methylpropyl]piperidine-1-carboxylic acid isopropyl ester (Preparation 17, 150 mg, 0.42 mmol) and [(3S,4S)-4-(2-oxopiperidin-1-yl)pyrrolidin-3-yl]carbamic acid tert-butyl ester (Preparation 41, 131 mg, 0.46 mmol) in DMSO (2.5 mL), under argon, was added DBU (65 μL, 0.42 mmol) and the mixture heated to 100° C. until no further reaction was observed. The mixture was partitioned between EtOAc and brine, then the organic phase was separated, dried (Na₂SO₄) and the solvent removed in vacuo. Purification by column chromatography (IH:EtOAc, 100:0, 80:40, 70:30, 50:50, 0:100 then EtOAc:MeOH, 9:1) afforded the title compound: RT=4.10 min; m/z (ES⁺)=603.3 [M+H]⁺.

### Preparation 79: [(3S,4S)-1-(5-{(R)-3-[1-(3-Isopropyl-[1,2,4]oxadiazol-5-yl)piperidin-4-yl]butoxy}-3-methylpyridin-2-yl)-4-(2-oxopiperidin-1-yl)pyrrolidin-3-yl]carbamic acid tert-butyl ester

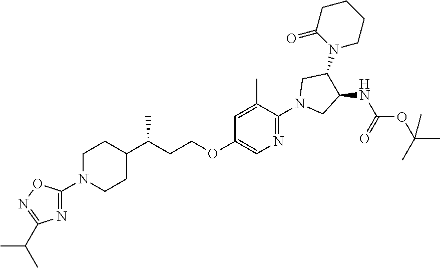

A combination of 2-bromo-5-{(R)-3-[1-(3-isopropyl-[1,2,4]oxadiazol-5-yl)piperidin-4-yl]butoxy}-3-methylpyridine (Preparation 18, 133 mg, 0.30 mmol), [(3S,4S)-4-(2-oxopiperidin-1-yl)pyrrolidin-3-yl]carbamic acid tert-butyl ester (Preparation 41, 103 mg, 0.37 mmol), sodium tert-butoxide (102 mg, 1.06 mmol), 2,8,9-triisobutyl-2,5,8,9-tetraaza-1-phosphabicyclo[3.3.3]undecane (10 mg, 0.03 mmol) and tris(dibenzylideneacetone)dipalladium (27 mg, 0.03 mmol) in dioxane (3.0 mL) was bubbled with argon for 15 min and the reaction was heated in a microwave reactor at 120° C. for 30 min. The mixture was filtered through a plug of celite, washing with DCM then MeOH, and the solvent was removed in vacuo. The resulting residue was taken up into DCM, washed with water and passed through a phase separator. Removal of the solvent in vacuo afforded the title compound: RT=3.42 min; m/z (ES⁺)=640.6 [M+H]⁺.

### Preparation 80: 4-((R)-3-{2-[(3R,4S)-3-tert-Butoxycarbonylamino-4-(2,5-difluorophenyl)pyrrolidin-1-yl]pyrimidin-5-yloxy}-1-methylpropyl)piperidine-1-carboxylic acid isopropyl ester

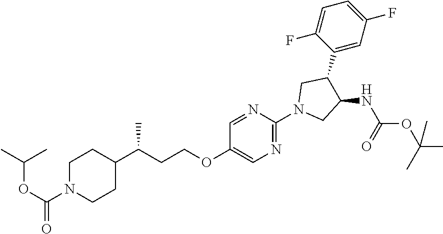

To a solution of 4-[(R)-3-(2-chloropyrimidin-5-yloxy)-1-methylpropyl]piperidine-1-carboxylic acid isopropyl ester (Preparation 17, 100 mg, 0.28 mmol) and [(3R,4S)-4-(2,5-difluorophenyl)pyrrolidin-3-yl]carbamic acid tert-butyl ester (Preparation 70, 167 mg, 0.56 mmol) in DMSO (4.0 mL), under argon, was added DBU (42 μL, 0.28 mmol) and the reaction heated to 80° C. for 64 h followed by stirring at r.t. for 64 h. The mixture was partitioned between EtOAc and brine. The organic phase was separated, dried (Na₂SO₄), and solvent removed in vacuo. Purification by column chromatography (DCM:MeOH, 100:0, 90:10, 80:20) afforded the title compound: RT=4.81 min; m/z (ES⁺)=618.3 [M+H]⁺.

### Preparation 81: 4-((R)-3-{2-[(trans)-3-tert-Butoxycarbonylamino-4-(2,4-difluorophenyl)pyrrolidin-1-yl]pyrimidin-5-yloxy}-1-methylpropyl)piperidine-1-carboxylic acid isopropyl ester

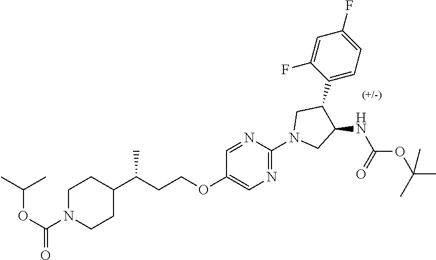

To a solution of 4-[(R)-3-(2-chloropyrimidin-5-yloxy)-1-methylpropyl]piperidine-1-carboxylic acid isopropyl ester (Preparation 17, 200 mg, 0.56 mmol) and [(trans)-4-(2,4-difluorophenyl)pyrrolidin-3-yl]carbamic acid tert-butyl ester (Preparation 60, 335 mg, 1.12 mmol) in DMSO (4.0 mL), under argon, was added DBU (84 μL, 0.56 mmol) and the reaction was heated to 80° C. in a sealed tube for 72 h. A further portion of [trans-4-(2,4-difluorophenyl)pyrrolidin-3-yl]carbamic acid tert-butyl ester (80 mg, 0.27 mmol) was added and the reaction continued until completion. The reaction mixture was cooled to r.t. and partitioned between EtOAc and brine. The organic phase was separated, dried (Na₂SO₄) and the solvent removed in vacuo. Purification by column chromatography (DCM:MeOH, 100:0, 95:5, 90:10) afforded the title compound: RT=4.86 min; m/z (ES⁺)=618.6 [M+H]⁺.

### Preparation 82: [(trans)-1-(5{(R)-3-[1-(5-Chloropyrimidin-2-yl)piperidin-4-yl]butoxy}pyrimidin-2-yl)-4-(2,5-difluorophenyl)pyrrolidin-3-yl]carbamic acid tert-butyl ester

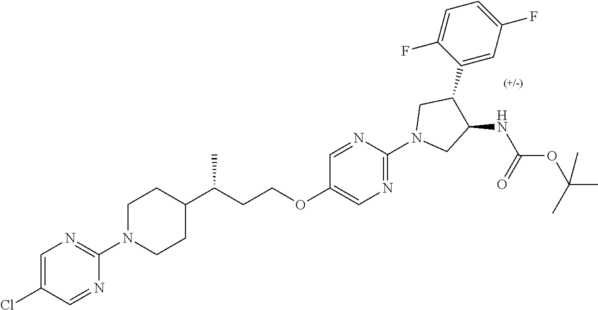

(R)-5-Chloro-2-{4-[1-methyl-3-(2-chloropyrimidin-5-yloxy)propyl]-piperidin-1-yl}pyrimidine (Preparation 4) was reacted with [(trans)-4-(2,5-difluorophenyl)pyrrolidin-3-yl]carbamic acid tert-butyl ester (Preparation 68), employing the procedure outlined in Preparation 77.The reaction mixture was partitioned between EtOAc and brine, then the organic phase was separated, washed with 0.1M citric acid, sat. NaHCO₃ solution, and dried (MgSO₄). Removal of the solvent in vacuo and purification by column chromatography (IH:EtOAc, 9:1) afforded the title compound: RT=5.24 min; m/z (ES⁺)=644.2 [M+H]⁺.

### Preparation 83: [(3R,4S)-4-(2,5-Difluorophenyl)-1-(6-{(R)-3-[1-(3-isopropyl-[1,2,4]oxadiazol-5-yl)piperidin-4-yl]butoxy}pyrimidin-4-yl)pyrrolidin-3-yl]carbamic acid tert-butyl ester

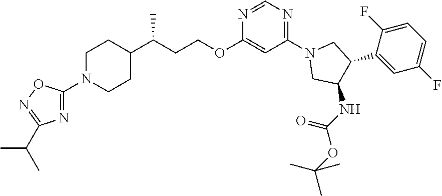

A mixture of (R)-4-chloro-6-{3-[1-(3-isopropyl-[1,2,4]oxadiazol-5-yl)piperidin-4-yl]butoxy}pyrimidine (Preparation 6, 40 mg, 0.1 mmol), [(3R,4S)-4-(2,5-difluorophenyl)-pyrrolidin-3-yl]carbamic acid tert-butyl ester (Preparation 70, 30 mg, 0.1 mmol) and triethylamine (50 μL, 0.15 mmol) in tert-butanol (1 mL) was heated in a microwave reactor at 140° C. for 30 min. The reaction mixture was partitioned between DCM and water and organic phase was separated, dried (MgSO₄), and the solvent removed in vacuo. Purification by preparative HPLC afforded the title compound: RT=4.52 min; m/z (ES⁺)=642.3 [M+H]⁺.

### Preparation 84: [(3S,4S)-1-(6-{(R)-3-[1-(3-Isopropyl-[1,2,4]oxadiazol-5-yl)piperidin-4-yl]butoxy}pyridine-3-yl)-4-(2-oxopiperidin-1-yl)pyrrolidin-3-yl]carbamic acid tert-butyl ester

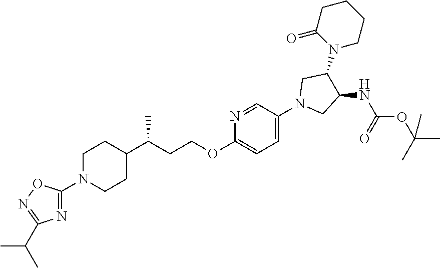

A combination of 5-bromo-2-{(R)-3-[1-(3-isopropyl-[1,2,4]oxadiazol-5-yl)piperidin-4-yl]butoxy}pyridine (Preparation 11, 150 mg, 0.35 mmol), [(3S,4S)-4-(2-oxopiperidin-1-yl)pyrrolidin-3-yl]carbamic acid tert-butyl ester (Preparation 41, 120 mg, 0.43 mmol), (2-biphenyl)di tert-butylphosphine (21 mg, 0.07 mmol), sodium tert-butoxide (41 mg, 0.43 mmol) and tris(dibenzylideneacetone)dipalladium (16 mg, 0.01 mmol) in toluene (2 mL) was bubbled with argon for 20 min before being heated to 70° C. for 16 h. The temperature was raised to 75° C. and stirring continued for a further 24 h. The reaction mixture was diluted with EtOAc (30 mL), washed with water (2×20 mL), sat. NaHCO₃ solution (20 mL), brine (20 mL) and dried (MgSO₄). Removal of the solvent in vacuo and purification by column chromatography (DCM:MeOH, 98:2, 96:4) afforded the title compound: RT=4.18 min; m/z (ES⁺)=626.4 [M+H]⁺.

### Preparation 85: [(3S,4S)-1-(5-{(R)-3-[1-(3-Isopropyl-[1,2,4]oxadiazol-5-yl)piperidin-4-yl]butoxy}pyrimidin-2-yl)-4-(4-methyl-2-oxopiperidin-1-yl)pyrrolidin-3-yl]carbamic acid tert-butyl ester

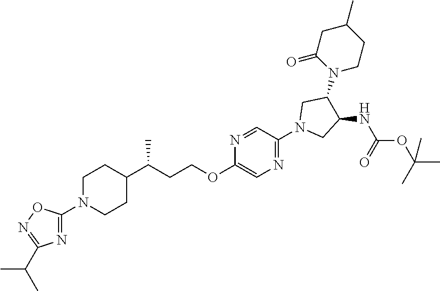

The title compound was prepared by reacting (R)-2-chloro-5-{3-[1-(3-isopropyl-[1,2,4]oxadiazol-5-yl)piperidin-4-yl]butoxy}pyrimidine (Preparation 7) with [(3S,4S)-4-(4-methyl-2-oxopiperidin-1-yl)pyrrolidin-3-yl]carbamic acid tert-butyl ester (Preparation 43) employing the procedure outlined in Preparation 88: RT=4.26 min m/z (ES⁺)=641.4 [M+H]⁺.

### Preparation 86: [(3S,4S)-1-(5-{(R)-3-[1-(3-Isopropyl-[1,2,4]oxadiazol-5-yl)piperidin-4-yl]butoxy}pyrimidin-2-yl)-4-((S)-4-methyl-2-oxopiperidin-1-yl)pyrrolidin-3-yl]carbamic acid tert-butyl ester

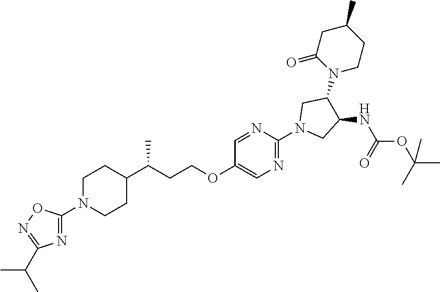

The title compound was afforded via chiral HPLC separation of [(3S,4S)-1-(5-{(R)-3-[1-(3-isopropyl-[1,2,4]oxadiazol-5-yl)piperidin-4-yl]butoxy}pyrimidin-2-yl)-4-(4-methyl-2-oxopiperidin-1-yl)pyrrolidin-3-yl]carbamic acid tert-butyl ester (Preparation 85): MTBE:THF 70:30, 12 ml/min, 250 nm.

### Preparation 87: [(3S,4S)-1-(5-{(R)-3-[1-(3-Isopropyl-[1,2,4]oxadiazol-5-yl)piperidin-4-yl]butoxy}pyrimidin-2-yl)-4-((R)-4-methyl-2-oxopiperidin-1-yl)pyrrolidin-3-yl]carbamic acid tert-butyl ester

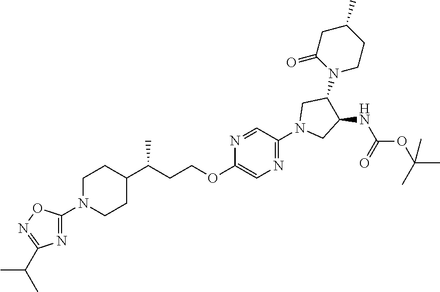

The title compound was afforded via chiral HPLC separation of [(3S,4S)-1-(5-{(R)-3-[1-(3-isopropyl-[1,2,4]oxadiazol-5-yl)piperidin-4-yl]butoxy}pyrimidin-2-yl)-4-(4-methyl-2-oxopiperidin-1-yl)pyrrolidin-3-yl]carbamic acid tert-butyl ester (Preparation 85): MTBE:THF 70:30, 12 ml/min, 250 nm.

### Preparation 88: [(3′S,4′S)-1′-(5-{(R)-3-[1-(3-Isopropyl-[1,2,4]oxadiazol-5-yl)piperidin-4-yl]butoxy}pyrimidin-2-yl)-2-oxo-[1,3]bipyrrolidinyl-4′-yl]carbamic acid tert-butyl ester

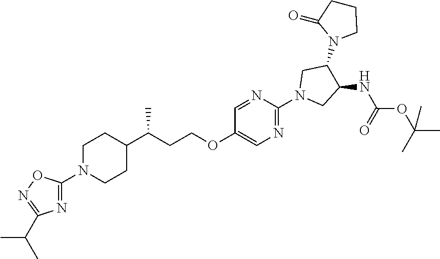

To a solution of (R)-2-chloro-5-{3-[1-(3-isopropyl-[1,2,4]oxadiazol-5-yl)piperidin-4-yl]butoxy}pyrimidine (Preparation 7, 128 mg, 0.34 mmol) and (3′S,4′S)-4′ -amino-1′-benzyl-[1,3′]bipyrrolidinyl-2-one (Preparation 44, 100 mg, 0.37 mmol) in DMSO (700 μL) was added DBU (50 μL, 0.34 mmol) and the reaction heated to 85° C. in a sealed tube until completion. Water (30 mL) was added and the mixture extracted with EtOAc (2×30 mL). Organic fractions were combined, washed with water (30 mL), sat. NaHCO₃ solution (30 mL), then brine (30 mL), and dried (MgSO₄). Removal of the solvent in vacuo and purification by column chromatography (DCM:MeOH, 100:0, 98:2) afforded the title compound: RT=3.96 min; m/z (ES⁺)=613.3 [M+H]⁺.

### Preparation 89: [(3S,4S)-4-(5,5-Difluoro-2-oxopiperidin-1-yl)-1-(5-{(R-3[1-(3-isopropyl[1,2,4]oxadiazol-5-yl)piperidin-4-yl]butoxy}pyrimidin-2-yl)pyrrolidin-3-yl]carbamic acid tert-butyl ester

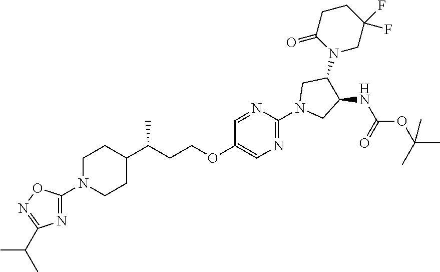

To a solution of (R)-2-chloro-5-{3-[1-(3-isopropyl-[1,2,4]oxadiazol-5-yl)piperidin-4-yl]butoxy}pyrimidine (Preparation 7, 197 mg, 0.62 mmol) and [(3S,4S)-4-(5,5-difluoro-2-oxopiperidin-1-yl)pyrrolidin-3-yl]carbamic acid tert-butyl ester (Preparation 45, 304 mg, 0.80 mmol) in DMSO (2 mL) was added DBU (90 mL, 0.62 mmol) and the reaction was heated to 85° C. for 30 h. Water (20 mL) was added and the crude mixture extracted with EtOAc (3×20 mL). The organic fractions were combined, washed with water (20 mL), sat. Na₂CO₃ solution (20 mL), then brine (30 mL), and dried (MgSO₄). Removal of the solvent in vacuo and purification by column chromatography (DCM:MeOH, 99:1, 97:3) afforded the title compound: RT=4.09 min m/z (ES⁺)=633.4 [M+H]⁺.

### Preparation 90: [(3S,4S)-1-(5-{3-[1-(3-Isopropyl-[1,2,4]oxadiazol-5-yl)piperidin-4-yl]propoxy}pyrimidin-2-yl)-4-(2-oxopiperidin-1-yl)pyrrolidin-3-yl]carbamic acid tert-butyl ester

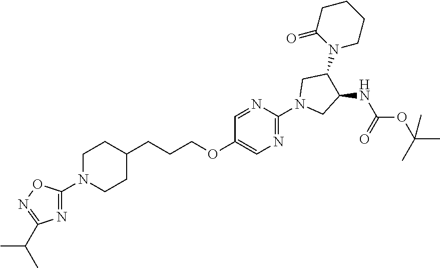

2-Chloro-5-{3-[1-(3-isopropyl-[1,2,4]oxadiazol-5-yl)piperidin-4-yl]propoxy}pyrimidine (Preparation 19, 48 mg, 0.13 mmol) was reacted with [(3S,4S)-4-(2-oxopiperidin-1-yl)pyrrolidin-3-yl]carbamic acid tert-butyl ester (Preparation 41, 34 mg, 0.26 mmol) employing the procedure outlined in Preparation 77. Water (40 mL) was added and the mixture extracted with EtOAc (30 mL). The organic layer was washed with water (25 mL), then brine (30 mL), and dried (MgSO₄). Removal of the solvent in vacuo and purification by column chromatography (DCM:MeOH, 98:2) afforded the title compound: RT=3.90 min; m/z (ES⁺)=613.4 [M+H]⁺.

### Preparation 91: [(3R,4S)-1-(5-{(R)-3-[1-(3-Isopropyl-[1,2,4]oxadiazol-5-yl)piperidin-4-yl]butoxy}pyrimidin-2-yl)-4-(2,4,5-trifluorophenyl)pyrrolidin-3-yl]carbamic acid tert-butyl ester

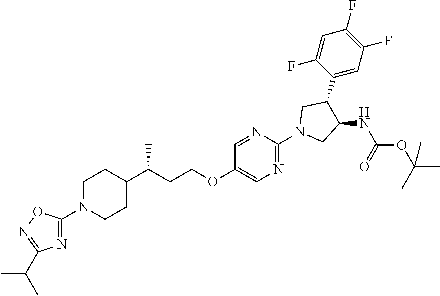

To a solution of (R)-2-chloro-5-{3-[1-(3-isopropyl-[1,2,4]oxadiazol-5-yl)piperidin-4-yl]butoxy}pyrimidine (Preparation 7, 150 mg, 0.44 mmol) and [(3R,4S)-4-(2,4,5-trifluorophenyl)pyrrolidin-3-yl]carbamic acid tert-butyl ester (Preparation 66, 150 mg, 0.47 mmol) in DMSO (0.8 mL) was added DBU (59 μL, 0.62 mmol) and the mixture was heated to 70° C. until no further reaction was observed. Water (25 mL) was added and the crude mixture extracted with EtOAc (5×15 mL). The organic fractions were combined, washed with water (20 mL), brine (40 mL), and dried (MgSO₄). Removal of the solvent in vacuo and purification by column chromatography (DCM:MeOH, 99:1) afforded the title compound: RT=4.80 min m/z (ES⁺)=660.4 [M+H]⁺.

### Preparation 92: [(3R,4S)-1-(5-{(R)-3-[1-(3-Ethyl-[1,2,4]oxadiazol-5-yl)piperidin-4-yl]butoxy}pyrimidin-2-yl)-4-(2,4,5-trifluorophenyl)pyrrolidin-3-yl]carbamic acid tert-butyl ester

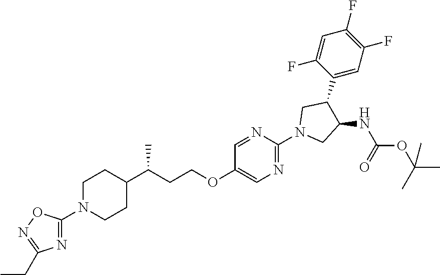

The title compound was prepared by reacting 2-chloro-5-{(R)-3-[1-(3-ethyl-[1,2,4]oxadiazol-5-yl)piperidin-4-yl]butoxy}pyrimidine (Preparation 21) with [(3R,4S)-4-(2,4,5-trifluorophenyl)pyrrolidin-3-yl]carbamic acid tert-butyl ester (Preparation 66) employing the procedure outlined in Preparation 91: RT=4.70 min m/z (ES⁺)=646.4 [M+H]⁺.

### Preparation 93: [(3R,4S)-4-(2,5-Difluorophenyl)-1-(5-{(R)-3-[1-(3-isopropyl-[1,2,4]oxadiazol-5-yl)piperidin-4-yl]butoxy}-3-methylpyridin-2-yl)pyrrolidin-3-yl]carbamic acid tert-butyl ester

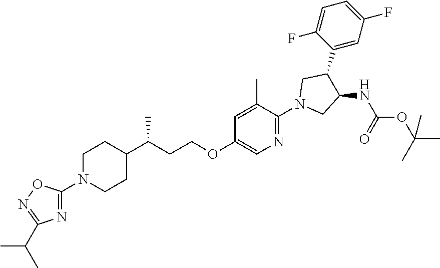

The title compound was prepared by reacting 2-bromo-5-{(R)-3-[1-(3-isopropyl-[1,2,4]oxadiazol-5-yl)piperidin-4-yl]butoxy}-3-methylpyridine (Preparation 18) with [(3R,4S)-4-(2,5-difluorophenyl)pyrrolidin-3-yl]carbamic acid tert-butyl ester (Preparation 70) employing the procedure outlined in Preparation 79: RT=3.57 min; m/z (ES⁺)=555.3 [M+H—(C₅H₈O₂)]⁺.

### Preparation 94: 4-((R)-3-{2-[(3R,4R)-3-tert-Butoxycarbonylamino-4-(2,5-difluorophenyl)piperidin-1-yl]pyrimidin-5-yloxy}-1-methylpropyl)piperidine-1-carboxylic acid isopropyl ester

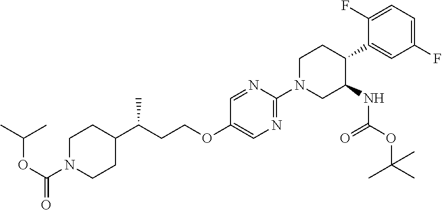

4-[(R)-3-(2-Chloropyrimidin-5-yloxy)-1-methylpropyl]piperidine-1-carboxylic acid isopropyl ester (Preparation 17, 139 mg, 0.36 mmol) was reacted with [(3R,4R)-4-(2,5-difluorophenyl)piperidin-3-yl]carbamic acid tert-butyl ester (Preparation 50, 94 mg, 0.30 mmol) employing the procedure outlined in Preparation 78. The mixture was diluted with EtOAc (50 mL) and the resulting solution was washed with sat. NaHCO₃ solution (50 mL), brine (50 mL), and dried (MgSO₄). Removal of the solvent in vacuo and purification by column chromatography (IH:EtOAc, 1:1) afforded the title compound: RT=5.09 min m/z (ES⁺)=362.4 [M+H]⁺.

### Preparation 95: [(3R,4R)-4-(2,5-Difluorophenyl)-1-(5-{(R)-3-[1-(3-isopropyl-[1,2,4]oxadiazol-5-yl)piperidin-4-yl]butoxy}pyrimidin-2-yl)piperidin-3-yl]carbamic acid tert-butyl ester

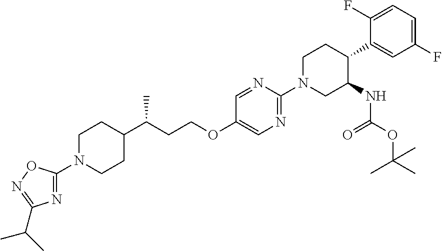

The title compound was prepared by reacting (R)-2-chloro-5-{3-[1-(3-isopropyl-[1,2,4]oxadiazol-5-yl)piperidin-4-yl]butoxy}pyrimidine (Preparation 7) with [(3R,4R)-4-(2,5-difluorophenyl)piperidin-3-yl]carbamic acid tert-butyl ester (Preparation 50) employing the procedure outlined in Preparation 94: RT=5.07 min m/z (ES⁺)=656.3 [M+H]⁺.

### Preparation 96: (trans)-3-(9H-Fluoren-9-ylmethoxycarbonylamino)-4-(2-fluorophenyl)pyrrolidine-1-carboxylic acid tert-butyl ester

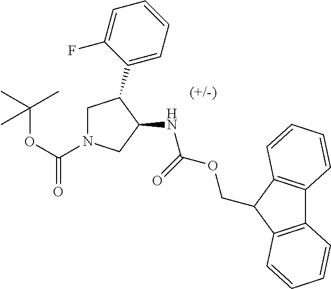

To a solution of (trans)-3-Amino-4-(2-fluorophenyl)pyrrolidine-1-carboxylic acid tert-butyl ester (2.00 g, 7.13 mmol) and triethylamine (1.59 mL, 11.40 mmol) in a combination of dioxane and water (2:1, 75 mL), cooled to 0° C., was added 9-fluorenylmethyl chloroformate (2.31 g, 8.92 mmol), and the reaction was allowed to warm to r.t. before stirring for 16 h. The mixture was diluted with EtOAc, then the solution washed with water, 1M HCl, sat. NaHCO₃ solution, brine and dried (MgSO₄). Removal of the solvent in vacuo and purification by column chromatography (IH:EtOAc, 90:10, 80:20, 70:30) afforded the title compound: RT=4.28 min m/z (ES⁺)=503.3 [M+H]⁺.

### Preparation 97: (3R,4S)-3-(9H-Fluoren-9-ylmethoxycarbonylamino)-4-(2-fluorophenyl)pyrrolidine-1-carboxylic acid tert-butyl ester

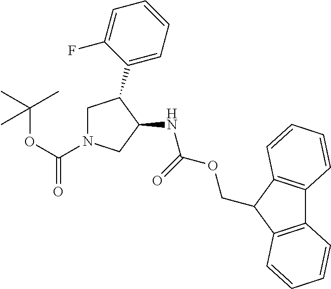

The title compound was afforded via chiral HPLC separation of (trans)-3-(9H-fluoren-9-ylmethoxycarbonylamino)-4-(2-fluorophenyl)pyrrolidine-1-carboxylic acid tert-butyl ester (Preparation 96): IH:CHCl₃:IPA:DEA 85:10:5:0.1, 15 ml/min, 270 nm, RT=9.4 min

### Preparation 98: [(trans)-4-(2-Fluorophenyl)pyrrolidin-3-yl]carbamic acid 9H-fluoren-9-ylmethyl ester hydrochloride

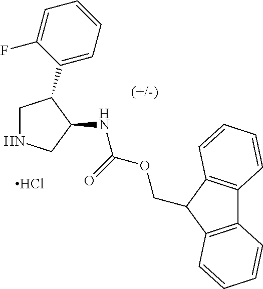

To a solution of (trans)-3-(9H-fluoren-9-ylmethoxycarbonylamino)-4-(2-fluorophenyl)pyrrolidine-1-carboxylic acid tert-butyl ester (Preparation 96, 1.50 g, 2.98 mmol) in dioxane (30 mL) was added a solution of HCl in dioxane (4M, 30 mL) and the reaction was stirred at r.t. for 16 h, after which time a precipitate formed. Et₂O was added to the mixture, until no further precipitation was observed, and the solvent was decanted. The residue was suspended in a further volume of Et₂O and stirred for 5 min before decanting the solvent. This process was repeated twice more and the resulting residue was concentrated in vacuo to afford the title compound: RT=2.82 min m/z (ES⁺)=403.1 [M+H]⁺.

### Preparation 99: [(3R,4S)-4-(2-Fluorophenyl)pyrrolidin-3-yl]carbamic acid 9H-fluoren-9-ylmethyl ester hydrochloride

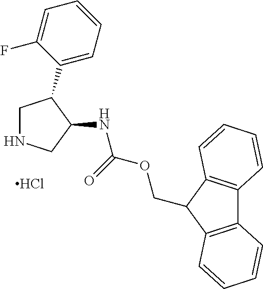

The title compound was prepared from (3R,4S)-3-(9H-fluoren-9-ylmethoxycarbonylamino)-4-(2-fluorophenyl)pyrrolidine-1-carboxylic acid tert-butyl ester (Preparation 97) employing the procedure outlined in Preparation 98: RT=2.82 min m/z (ES⁺)=403.1 [M+H]⁺.

### Preparation 100: [(3S,4S)-1-(6-{(R)-3-[1-(3-Isopropyl-[1,2,4]oxadiazol-5-yl)piperidin-4-yl]butoxy}pyridazin-3-yl)-4-(2-oxopiperidin-1-yl)pyrrolidin-3-yl]carbamic acid tert-butyl ester

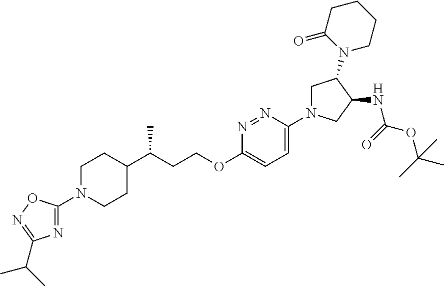

3-Chloro-6-{(R)-3-[1-(3-isopropyl-[1,2,4]oxadiazol-5-yl)piperidin-4-yl]butoxy}-pyridazine (Preparation 14, 130 mg, 0.34 mmol) was reacted with [(3S,4S)-4-(2-oxopiperidin-1-yl)pyrrolidin-3-yl]carbamic acid tert-butyl ester (Preparation 41, 106 mg, 0.38 mmol) employing the procedure outlined in Preparation 78. The mixture was diluted with EtOAc and the resulting solution washed with water. The aqueous phase was separated, extracted with EtOAc, then the organic fractions were combined, washed with water (×5), brine, and dried (MgSO₄). Removal of the solvent in vacuo and purification by column chromatography (DCM:MeOH, 95:5) afforded the title compound: RT=3.62 min m/z (ES⁺)=627.4 [M+H]⁺.

### Preparation 101: 4-((R)-1-Methyl-3-oxopropyl)piperidine-1-carboxylic acid isopropyl ester

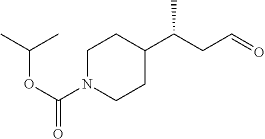

To a solution of 4-(R)-3-hydroxy-1-methylpropyl)piperidine-1-carboxylic acid isopropyl ester (Preparation 15, 2.00 g, 7.41 mmol) in DCM (50 mL) was added dess-martin periodinane (3.77 g, 8.90 mmol) and the mixture was stirred at r.t. for 16 h. The reaction was quenched by the addition of sat. NaHCO₃ solution (50 mL), and solid sodium thiosulfate (2.5 g) was added before allowing the mixture to stir for 16 h. The organic phase was separated, washed with brine, dried (MgSO₄), and the solvent removed in vacuo. Purification by column chromatography (IH:EtOAc, 70:30) afforded the title compound: RT=3.48 min; m/z (ES⁺)=242.2 [M+H]⁺.

### Preparation 102: 4-((R)-1-Methylbut-3-ynyl)piperidine-1-carboxylic acid isopropyl ester

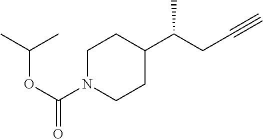

A solution of n-butyllithium (1M in THF, 3.5 mL, 3.5 mmol) in an oven-dried flask, under argon, was cooled to −78° C., and trimethylsilyldiazomethane (2M in Et₂O, 1.72 mL, 3.44 mmol) was added. The mixture was stirred at this temperature for 45 min before the slow addition of 4-((R)-1-methyl-3-oxopropyl)piperidine-1-carboxylic acid isopropyl ester (Preparation 101, 830 mg, 3.44 mmol) in THF (20 mL). The mixture was stirred for 30 min at −78° C., and then allowed to warm to 0° C. for 30 min. The reaction was quenched by addition of sat. NH₄Cl solution, then diluted with Et₂O. The organic phase was separated, washed with 1M citric acid, water, brine, and dried (MgSO₄). Purification by column chromatography (IH:EtOAc, 80:20, 70:30) afforded the title compound: RT=4.03 min; m/z (ES⁺)=238.1 [M+H]⁺.

### Preparation 103: [(3S,4S)-1-(5-Bromopyrimidin-2-yl)-4-(2-oxopiperidin-1-yl)pyrrolidin-3-yl]carbamic acid tert-butyl ester

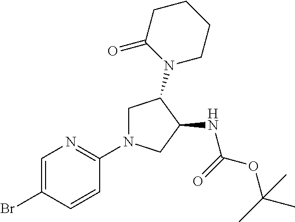

5-Bromo-2-chloropyrimidine was reacted with [(3S,4S)-4-(2-oxopiperidin-1-yl)pyrrolidin-3-yl]carbamic acid tert-butyl ester (Preparation 41) employing the procedure outlined in Preparation 78. The crude reaction mixture was cooled to r.t. and diluted with EtOAc. The organic solution was washed with water (×3), 1M HCl, sat. NaHCO₃ solution, brine, and dried (MgSO₄). Removal of the solvent in vacuo and purification by column chromatography (IH:EtOAc, 40:60, 20:80) afforded the title compound: RT=3.45 min; m/z (ES⁺)=440.1, 442.1 [M+H]⁺.

### Preparation 104: 4-((R)-4-{2-[(3S,4S)-3-tert-Butoxycarbonylamino-4-(2-oxopiperidin-1-yl)pyrrolidin-1-yl]pyrimidin-5-yl}-1-methylbut-3-ynyl)piperidine-1-carboxylic acid isopropyl ester

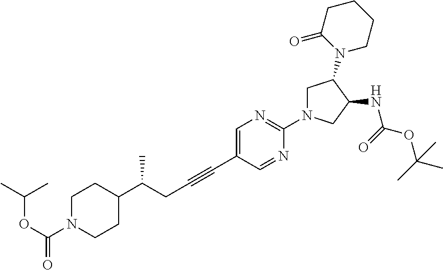

To a solution of [(3S,4S)-1-(5-bromopyrimidin-2-yl)-4-(2-oxopiperidin-1-yl)pyrrolidin-3-yl]carbamic acid tert-butyl ester (Preparation 103, 100 mg, 0.23 mmol) in DMF (0.4 mL) was added a solution of 4-((R)-1-methylbut-3-ynyl)piperidine-1-carboxylic acid isopropyl ester (Preparation 102, 81 mg, 0.34 mmol) and triethylamine (0.6 mL) in DMF (1.5 mL) and the resulting mixture was de-gassed with argon for 20 min. Copper iodide (9 mg, 0.04 mmol) and tetrakis(triphenylphosphine)palladium(0) (26 mg, 0.02 mmol) were added and the mixture was heated to 70° C. for 3 h before being heated to 80° C. for 16 h. A further portion of 4-((R)-1-methylbut-3-ynyl)piperidine-1-carboxylic acid isopropyl ester (Preparation 102, 81 mg, 0.34 mmol) in DMF (1.0 mL) was added and the temperature raised to 110° C. for 14 h before allowing the reaction to stir at r.t. for 60 h. The mixture was diluted with EtOAc, washed with water (×5), 1M citric acid, sat. NaHCO₃ solution, then brine, and dried (MgSO₄). Removal of the solvent in vacuo and purification by column chromatography (DCM:EtOAc, 1:1) afforded the title compound: RT=4.35 min; m/z (ES⁺)=597.3 [M+H]⁺.

### Preparation 105: 4-((R)-4-{2-[(3S,4S)-3-tert-butoxycarbonylamino-4-(2-oxopiperidin-1-yl)pyrrolidin-1-yl]pyrimidin-5-yl}-1-methylbutyl)piperidine-1-carboxylic acid isopropyl ester

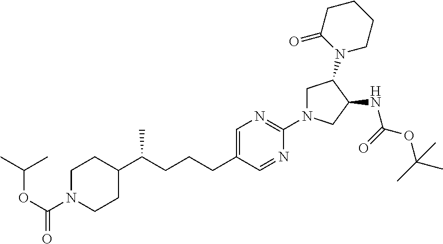

A solution of 4-((R)-4-{2-[(3S,4S)-3-tert-butoxycarbonylamino-4-(2-oxopiperidin-1-yl)pyrrolidin-1-yl]pyrimidin-5-yl}-1-methylbut-3-ynyl)piperidine-1-carboxylic acid isopropyl ester (Preparation 104, 73 mg, 0.12 mmol) in MeOH (2.4 mL) was passed through an H-Cube apparatus (10% pd/C Catcart 30, full hydrogen mode, r.t) at a flow rate of 3 mL per min. Removal of the solvent in vacuo and purification by column chromatography (DCM:MeOH, 95:5) afforded the title compound: RT=4.28 min; m/z (ES⁺)=601.5 [M+H]⁺.

### Preparation 106: [(trans)-4-(2,4-Difluorophenyl)-1-(5-{(R)-3-[1-(3-isopropyl-[1,2,4]oxadiazol-5-yl)piperidin-4-yl]butoxy}pyrimidin-2-yl)pyrrolidin-3-yl]carbamic acid tert-butyl ester

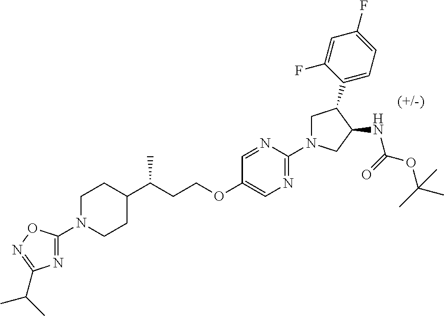

To a solution of (R)-2-Chloro-5-{3-[1-(3-isopropyl-[1,2,4]oxadiazol-5-yl)piperidin-4-yl]butoxy}pyrimidine (Preparation 7, 200 mg, 0.53 mmol) and [(trans)-4-(2,4-difluorophenyl)pyrrolidin-3-yl]carbamic acid tert-butyl ester (Preparation 60, 314 mg, 1.05 mmol) in DMSO (4.0 mL), under argon, was added DBU (81 μL, 0.56 mmol) and the reaction heated to 80° C. in a sealed tube for 72 h. The mixture was cooled to r.t. and partitioned between EtOAc and brine. The organic phase was separated, washed with brine, dried (Na₂SO₄) and the solvent removed in vacuo. Purification by column chromatography (DCM:MeOH, 100:0, 99:1, 98:2) afforded the title compound: RT=4.82 min; m/z (ES⁺)=642.3 [M+H]⁺.

### Preparation 107: 4-(1-Ethoxycarbonylethyl)-3,6-dihydro-2H-pyridine-1-carboxylic acid tert-butyl ester

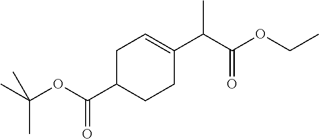

A solution of triethyl-2-phosphonopropionate (9.48 mL, 44.21 mmol) in THF (150 mL), under an atmosphere of argon, was cooled to 0° C. Sodium hydride (60% in mineral oil, 1.92 g, 47.89 mmol) was added, portionwise, over 10 min and the resulting reaction stirred at this temperature for 30 min. A solution of 4-oxopiperidine-1-carboxylic acid tert-butyl ester (7.34 g, 36.84 mmol) in THF (50 mL) was added to the mixture via cannula, over 10 min and the reaction was allowed to warm to r.t. before being heated to 60° C. for 2 h. The mixture was cooled, and the solvent concentrated in vacuo, before partitioning the residue between EtOAc (200 mL) and water (200 mL). The organic phase was separated and washed with water (100 mL), then the aqueous fractions were combined and extracted with EtOAc (100 mL). The resulting organic fractions were combined, washed with brine (100 mL), dried (MgSO₄) and the solvent removed in vacuo to afford the title compound: RT=3.96 min; m/z (ES⁺)=184.1 [M+H—(C₅H₈O₂)]⁺.

### Preparation 108: 4-(1-Ethoxycarbonylethyl)piperidine-1-carboxylic acid tert-butyl ester

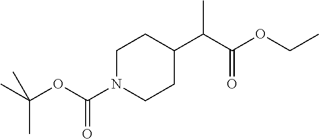

To a solution of 4-(1-ethoxycarbonylethyl)-3,6-dihydro-2H-pyridine-1-carboxylic acid tert-butyl ester (Preparation 107, 10.23 g, 36.10 mmol) in EtOH (150 mL) was added a slurry of palladium on carbon (10 Mol %, 4.61 g, 4.33 mmol) in water (15 mL) and the reaction was stirred under an atmosphere of hydrogen (atmospheric pressure) for 16 h. The mixture was filtered through a plug of celite, washing with EtOH (100 mL) and the filtrate concentrated in vacuo. The residue was re-dissolved in DCM, passed through a phase separater, and the solvent removed in vacuo to afford the title compound: RT=4.05 min; m/z (ES⁺)=186.1 [M+H—(C₅H₈O₂)]⁺.

### Preparation 109: 4-(2-Hydroxy-1-methylethyl)piperidine-1-carboxylic acid tert-butyl ester

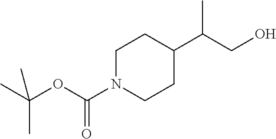

A solution of 4-(1-ethoxycarbonylethyl)piperidine-1-carboxylic acid tert-butyl ester (Preparation 108, 5.65 g, 19.80 mmol) in THF (200 mL), under argon, was cooled to 0° C. LAH (1.50 g, 39.59 mmol) was added, portionwise over 15 min, then the mixture was stirred at this temperature for 45 min. The reaction was quenched by the cautious addition of water (1.5 mL), followed by 15% NaOH solution (1.5 mL), and finally more water (4.5 mL). The resulting suspension was diluted with Et₂O (100 mL), stirred for 1 h then filtered, and the filtrate concentrated in vacuo. Purification by column chromatography (IH:EtOAc, 60:40, 50:50, 40:60) afforded the title compound: ¹H NMR δ_{H} (400 MHz, CDCl₃): 4.21-4.06 (m, 2H), 3.65-3.47 (m, 2H), 2.74-2.59 (m, 2H), 1.65-1.50 (m, 3H), 1.46 (s, 9H), 1.32-1.13 (m, 3H), 0.91 (d, J=6.6 Hz, 3H)

### Preparation 110: 2-Piperidin-4-ylpropan-1-ol hydrochloride

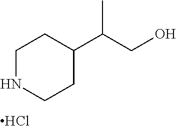

A solution of 4-(2-hydroxy-1-methylethyl)piperidine-1-carboxylic acid tert-butyl ester (Preparation 109, 7.77 g, 31.93 mmol) in dioxane (30 mL), under argon, was cooled to 0° C. HCl in dioxane (4M, 24 mL, 95.79 mmol) was added and the reaction stirred at this temperature for 3 h. Removal of the solvent in vacuo afforded the title compound: ¹H NMR δ_{H} (400 MHz, DMSO-d₆): 8.94 (br. s., 1H), 8.58 (br. s., 1H), 3.36-3.18 (m, 4H), 2.88-2.70 (m, 2H), 1.75-1.64 (m, 2H), 1.62-1.53 (m, 1H), 1.52-1.31 (m, 3H), 0.80 (d, J=7.0 Hz, 3H)

### Preparation 111: 4-(2-Hydroxy-1-methylethyl)piperidine-1-carboxylic acid isopropyl ester

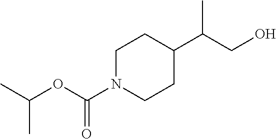

To a suspension of 2-piperidin-4-ylpropan-1-ol hydrochloride (Preparation 110, 1.00 g, 5.57 mmol) in DCM (15 mL), under argon, was added triethylamine (1.63 mL, 11.69 mmol) and the mixture was cooled to 0° C. Isopropyl chloroformate (1M in toluene, 6.12 mL, 6.12 mmol) was added, dropwise, over 5 min then the reaction was allowed to warm to r.t. and stirred for 16 h. The mixture was diluted with DCM (60 mL), washed with 1M HCl solution (2×50 mL), NaHCO₃ solution (50 mL) then brine (50 mL), and dried (MgSO₄). Purification by column chromatography (IH:EtOAc, 40:60) afforded the title compound: RT=3.04 min; m/z (ES⁺)=230.2 [M+H]⁺.

### Preparation 112: 4-[2-(2-Chloropyrimidin-5-yloxy)-1-methylethyl]piperidine-1-carboxylic acid isopropyl ester

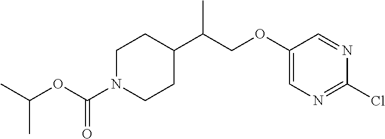

To a solution of 4-(2-hydroxy-1-methylethyl)piperidine-1-carboxylic acid isopropyl ester (Preparation 111, 400 mg, 1.74 mmol) in DCM (8 mL), under argon, was added triethylamine (316 μL, 2.27 mmol) and the mixture was cooled to 0° C. Methanesulfonyl chloride (163 μL, 2.09 mmol) was added before stirring the reaction at this temperature for 1 h. The mixture was diluted with DCM (50 mL), washed with 1M HCl solution (50 mL), sat. NaHCO₃ solution (50 mL), then brine (50 mL), and dried (MgSO₄). Removal of the solvent in vacuo afforded the intermediate product 4-(2-methanesulfonyloxy-1-methylethyl)piperidine-1-carboxylic acid isopropyl ester: RT=3.49 min; m/z (ES⁺)=308.1 [M+H]⁺. To a solution of the product in THF (8 mL), under argon, was added 2-chloropyrimidin-5-ol (249 mg, 1.91 mmol) followed by potassium carbonate (600 mg, 4.34 mmol), and the reaction was warmed to 50° C. for 16 h. DMF (2 mL) was added and the reaction heated to 55° C. for 3 h. A further portion of DMF (2 mL) was added and heating continued at 60° C. for 3 h. Further DMF (2 mL) was added and the reaction stirred at 50° C. for 10 h before being cooled to r.t. and the solvent removed in vacuo. The crude residue was partitioned between EtOAc (50 mL) and water (100 mL) and the organic phase was separated. The aqueous phase was extracted with EtOAc (50 mL) then the organic fractions were combined, washed with sat. NaHCO₃ solution (50 mL), brine (50 mL), and dried (MgSO₄). Removal of the solvent in vacuo afforded the title compound: ¹H NMR δ_{H} (400 MHz, CDCl₃): 8.29 (s, 2H), 4.98-4.88 (m, 1H), 4.29-4.12 (m, 2H), 4.02-3.86 (m, 2H), 2.78-2.64 (m, 2H), 1.96-1.84 (m, 1H), 1.70-1.59 (m, 3H), 1.37-1.319 (m, 8H), 1.04 (d, J=7.0 Hz, 3H).

### Example 1

### 1-[(3S,4S)-4-Amino-1-(5-{(R)-3-[1-(5-chloropyrimidin-2-yl)piperidin-4-yl]butoxy}pyrimidin-2-yl)pyrrolidin-3-yl]piperidin-2-one

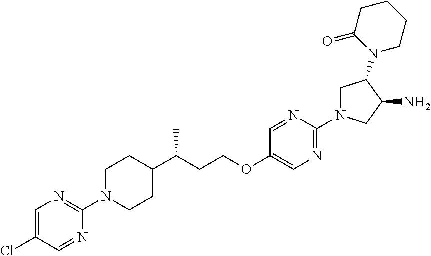

A combination of (R)-5-chloro-2-{4-[1-methyl-3-(2-chloropyrimidin-5-yloxy)-propyl]piperidin-1-yl}pyrimidine (Preparation 4, 160 mg, 0.42 mmol), [(3S,4S)-4-(2-oxopiperidin-1-yl)pyrrolidin-3-yl]carbamic acid tert-butyl ester (Preparation 41, 148 mg, 0.53 mmol) and DBU (160 mg, 1.05 mmol) in DMSO (2 mL) was heated to 100° C. for 16 h. The mixture was diluted with water and organics were subsequently extracted into DCM (×3) and dried (MgSO₄).Removal of the solvent in vacuo followed by purification by column chromatography (IH:IPA, 100:0, 85:15) afforded [(3S,4S)-1-(5-{(R)-3-[1-(5-chloropyrimidin-2-yl)piperidin-4-yl]butoxy}pyrimidin-2-yl)-4-(2-oxopiperidin-1-yl)pyrrolidin-3-yl]carbamic acid tert-butyl ester: RT=4.67 min; m/z (ES⁺)=629.3 [M+H]⁺. The residue was dissolved in DCM (5 mL), then TFA (1 mL) was added before stirring the mixture for 20 min. The reaction was quenched with sat. Na₂CO₃ solution and organics were extracted into DCM. The organic layer was washed with brine then dried (MgSO₄) and solvent was removed in vacuo. Purification by column chromatography (DCM:MeOH, 100:0, 85:15) afforded the title compound: RT=2.93 min; m/z (ES⁺)=529.6 [M+H]⁺.

### Example 2

### 1-[(3S,4S)-4-Amino-1-(5-{(R)-3-[1-(3-isopropyl-[1,2,4]oxadiazol-5-yl)piperidin-4-yl]butoxy}pyridin-2-yl)pyrrolidin-3-yl]piperidin-2-one p-toluenesulfonic acid salt

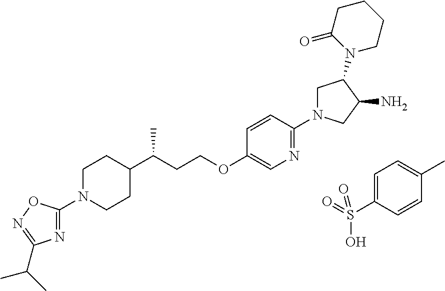

To a solution of (R)-2-bromo-5-{3-[1-(3-isopropyl-[1,2,4]oxadiazol-5-yl)piperidin-4-yl]butoxy}pyridine (Preparation 5, 200 mg, 0.47 mmol) in dioxane (5 mL) was added [(3S,4S)-4-(2-oxopiperidin-1-yl)pyrrolidin-3-yl]carbamic acid tert-butyl ester (Preparation 41, 160 mg, 0.56 mmol), 2,8,9-triisobutyl-2,5,8,9-tetraaza-1-phosphabicyclo-[3.3.3]undecane (16.2 mg, 0.05 mmol), potassium tert-butoxide (159 mg, 1.65 mmol) and tris-(dibenzylideneacetone)-dipalladium (43 mg, 0.05 mmol). The mixture was bubbled with argon for 30 min then heated in a microwave reactor at 120° C. for 60 min. The mixture was diluted with DCM, then washed with sat. NaHCO₃ solution, brine, and dried (MgSO₄). The solvent was removed in vacuo and the residue was purified by column chromatography (DCM:MeOH). Further purification by chiral HPLC afforded the title compound as the free amine. The product was dissolved in DCM and a solution of TsOH (1eq.) in MeOH was added. Removal of the solvent in vacuo afforded the title compound: RT=2.59 min; m/z (ES⁺)=526.5 [M+H]⁺.

### Example 3

### 1-[(3S,4S)-4-Amino-1-(6-{(R)-3-[1-(3-isopropyl-[1,2,4]oxadiazol-5-yl)piperidin-4-yl]butoxy}pyrimidin-4-yl)pyrrolidin-3-yl]piperidin-2-one

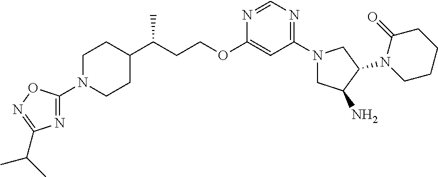

A combination of (R)-4-chloro-6-{3-[1-(3-isopropyl-[1,2,4]oxadiazol-5-yl)piperidin-4-yl]butoxy}pyrimidine (Preparation 6, 142 mg, 0.37 mmol), [(3S,4S)-4-(2-oxopiperidin-1-yl)pyrrolidin-3-yl]carbamic acid tert-butyl ester (Preparation 41, 106 mg, 0.37 mmol) and triethylamine (104 μL, 0.75 mmol) in tert-butanol (4 mL) was heated in a microwave reactor at 145° C. for 1 h. The reaction solvent was removed in vacuo and the resulting residue was re-dissolved in DCM. The organic solution was washed with water, then brine, and dried (MgSO₄). Removal of the solvent in vacuo afforded [(3S,4S)-1-(6-{(R)-3-[1-(3-isopropyl-[1,2,4]oxadiazol-5-yl)piperidin-4-yl]butoxy}pyrimidin-4-yl)-4-(2-oxopiperidin-1-yl)pyrrolidin-3-yl]carbamic acid tert-butyl ester: RT=3.67 min; m/z (ES⁺)=627.6 [M+H]⁺. The residue was dissolved in DCM (5 mL) and cooled to 0° C. before adding TFA (1 mL) and stirring for 2 h. The reaction was quenched with sat. Na₂CO₃ solution and organics extracted into DCM. The organic layer was washed with brine then dried (MgSO₄) and solvent was removed in vacuo. Purification by column chromatography (DCM:MeOH, 100:0, 95:5) afforded the title compound: RT=2.79 min; m/z (ES⁺)=527.5 [M+H]⁺.

### Example 4

### 1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]piperidin-2-one p-toluenesulfonic acid salt

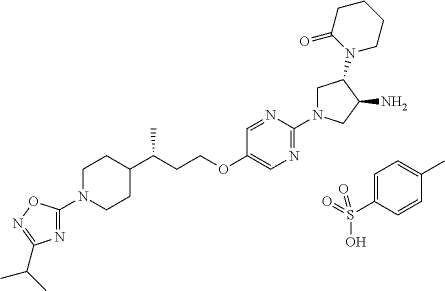

A combination of (R)-2-chloro-5-{3-[1-(3-isopropyl-[1,2,4]oxadiazol-5-yl)piperidin-4-yl]butoxy}pyrimidine (Preparation 7, 290 mg, 0.76 mmol), [(3S,4S)-4-(2-oxopiperidin-1-yl)pyrrolidin-3-yl]carbamic acid tert-butyl ester (Preparation 41, 270 mg, 0.95 mmol) and DBU (290 mg, 1.90 mmol) in DMSO (2 mL) was heated to 100° C. for 16 h. The mixture was diluted with water (50 mL) and extracted with DCM (×3). The combined organic fractions were washed with brine and dried (MgSO₄). Removal of the solvent in vacuo followed by purification by column chromatography (DCM:MeOH, 97:3) afforded [(3S,4S)-1-(5-{(R)-3-[1-(3-isopropyl-[1,2,4]oxadiazol-5-yl)piperidin-4-yl]butoxy}pyrimidin-2-yl)-4-(2-oxopiperidin-1-yl)pyrrolidin-3-yl]carbamic acid tert-butyl ester: RT=4.05 min; m/z (ES⁺)=627.4 [M+H]⁺. The residue was dissolved in DCM (5 mL), then TFA (1 mL) was added before stirring the mixture for 90 min. The reaction was quenched with sat. NaHCO₃ solution and organics were extracted into DCM. The organic layer was washed with brine then dried (MgSO₄) and solvent was removed in vacuo. Purification by column chromatography (DCM:MeOH, 100:0, 93:7) afforded the title compound as the free amine. The product was dissolved in DCM and a solution of TsOH (1eq.) in MeOH was added. Removal of the solvent in vacuo afforded the title compound: RT=2.73 min; m/z (ES⁺)=527.3 [M+H]⁺.

### Example 5

### 1-[3S,4S)-4-Amino-1-(5-{(R)-3-[1-(3-isopropyl-[1,2,4]oxadiazol-5-yl)piperidin-4-yl]butoxy}-4-methylpyrimidin-2-yl)pyrrolidin-3-yl]piperidin-2-one p-toluenesulfonic acid salt

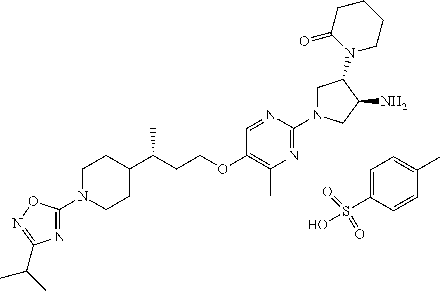

To a solution of [(3S,4S)-1-(5-{(R)-3-[1-(3-isopropyl-[1,2,4]oxadiazol-5-yl)piperidin-4-yl]butoxy}-4-methylpyrimidin-2-yl)-4-(2-oxopiperidin-1-yl)pyrrolidin-3-yl]carbamic acid tert-butyl ester (Preparation 71, 225 mg, 0.35 mmol) in DCM (6 mL) under argon, cooled to 0° C., was added TFA (1.5 mL) over 10 min and the reaction was allowed to stir at r.t. for 16 h. The reaction was diluted with DCM and washed with sat. NaHCO₃ solution, then dried (MgSO₄). Removal of the solvent in vacuo and purification by column chromatography (DCM:MeOH:NH₄OH, 100:0:0, 98:2:0, 97:3:0, 90:10:0, 90:10:1, 80:20:1) afforded the title compound as the free amine. The product was dissolved in DCM and a solution of TsOH (1eq) in MeOH was added. Removal of the solvent in vacuo afforded the title compound: RT=2.84 min; m/z (ES⁺)=541.3 [M+H]⁺.

### Example 6

### 1-[(3S,4S)-4-Amino-1-(2-{(R)-3-[1-(3-isopropyl-[1,2,4]oxadiazol-5-yl)piperidin-4-yl]butoxy}pyrimidin-5-yl)pyrrolidin-3-yl]piperidin-2-one hydrochloride

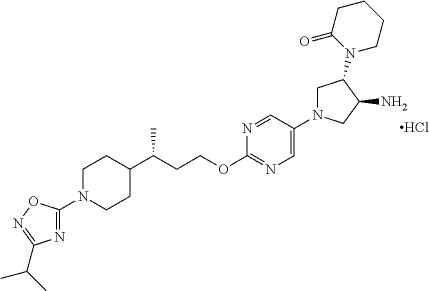

To a solution of [(3S,4S)-1-(2-{(R)-3-[1-(3-isopropyl-[1,2,4]oxadiazol-5-yl)piperidin-4-yl]butoxy}pyrimidin-5-yl)-4-(2-oxopiperidin-1-yl)pyrrolidin-3-yl]carbamic acid tert-butyl ester (Preparation 72, 14 mg, 0.02 mmol) in dioxane (0.2 mL) was added a solution of HCl in dioxane (4M, 0.2 mL) and the mixture was stirred at r.t. for 16 h. The reaction solvent was concentrated in vacuo and Et₂O was added. The mixture was stirred for 2 min and the Et₂O was decanted. This process was repeated four times to afford the title compound: RT=2.67 min; m/z (ES⁺)=527.3 [M+H]⁺.

The following compounds were prepared by treating the appropriate tert-butyl carbamate protected amine with 4M HCl in dioxane employing the procedure outlined in Example 6:

| Ex. | Structure | Name | LCMS |
|---|---|---|---|
| 7 | 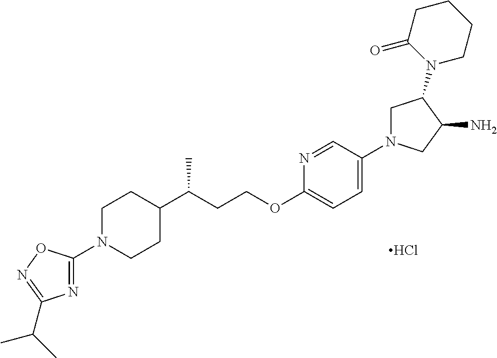 | 1-[(3S,4S)-4-Amino- 1-(6-{(R)-3-[1-(3- isopropyl- [1,2,4]oxadiazol-5- yl)piperidin-4- yl]butoxy}pyridin-3- yl)pyrrolidin-3- yl]piperidin-2-one hydrochloride | RT = 2.75 min; m/z (ES⁺) = 526.4 [M + H]⁺ |
| 8 | 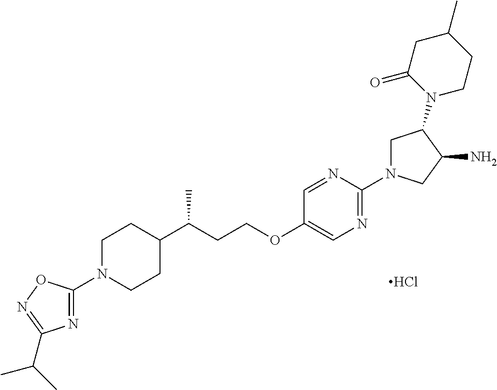 | 1-[(3S,4S)-4-Amino- 1-(5{(R)-3-[1-(3- isopropyl- [1,2,4]oxadiazol-5- yl)piperidin-4- yl]butoxy}pyrimidin- 2-yl)pyrrolidin-3-yl]- 4-methylpiperidin-2- one hydrochloride | RT = 2.84 min; m/z (ES⁺) = 541.3 [M + H]⁺ |
| 9 | 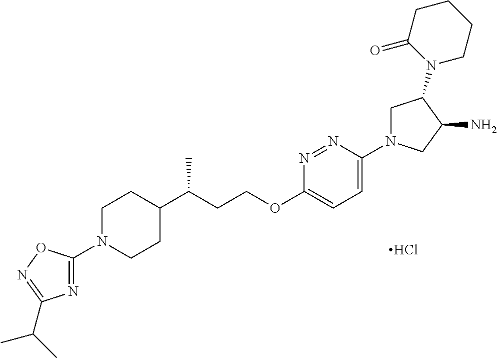 | 1-[(3S,4S)-4-Amino- 1-(6-{(R)-3-[1-(3- isopropyl- [1,2,4]oxadiazol-5- yl)piperidin-4- yl]butoxy}pyridazin- 3-yl)pyrrolidin-3- yl]piperidin-2-one hydrochloride | RT = 2.50 min; m/z (ES⁺) = 527.3 [M + H]⁺ |

### Example 10

### 1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]-5-methylpiperidin-2-one

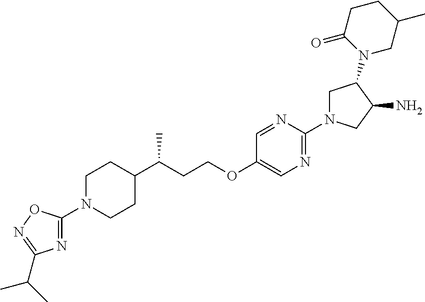

To a solution of [(3S,4S)-1-(5-{(R)-3-[1-(3-isopropyl-[1,2,4]oxadiazol-5-yl)piperidin-4-yl]butoxy}pyrimidin-2-yl)-4-(5-methyl-2-oxopiperidin-1-yl)pyrrolidin-3-yl]carbamic acid tert-butyl ester (Preparation 74, 14 mg, 0.02 mmol) in DCM (2.5 mL) under argon, was added TFA (0.5 mL) and the reaction was allowed to stir at r.t. for 16 h. The reaction was quenched with sat. Na₂CO₃ solution (30 mL) and extracted with EtOAc (30 mL). The organic phase was washed with brine and dried (MgSO₄). Removal of the solvent in vacuo followed by purification by column chromatography (DCM:MeOH:NH₄OH, 96:4:0.4) afforded the title compound: RT=2.81 min; m/z (ES⁺)=541.4 [M+H]⁺.

The following examples were prepared by treating the appropriate tert-butyl carbamate protected amine with TFA employing a procedure similar to that outlined in Example 10:

| Ex. | Structure | Name | LCMS |
|---|---|---|---|
| 11 | 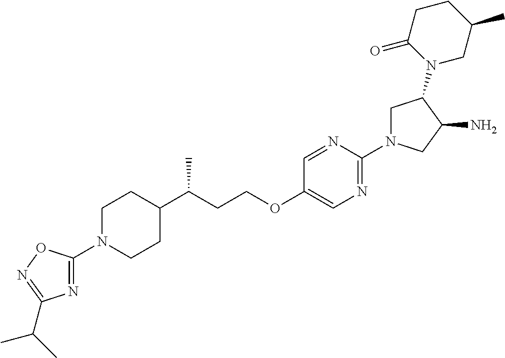 | (R)-1-[(3S,4S)-4-Amino- 1-(5-{(R)-3-[1-(3- isopropyl- [1,2,4]oxadiazol-5- yl)piperidin-4- yl]butoxy}pyrimidin-2- yl)pyrrolidin-3-yl]-5- methylpiperidin-2-one | RT = 2.74 min; m/z (ES⁺) = 541.4 [M + H]⁺ |
| 12 | 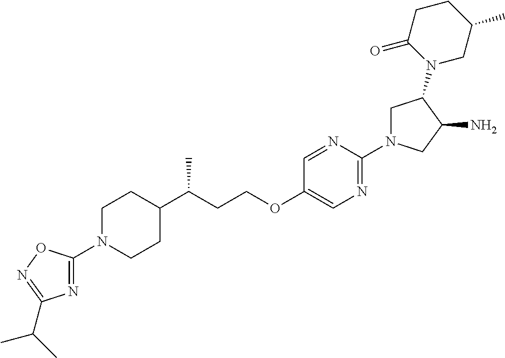 | (S)-1-[(3S,4S)-4-Amino- 1-(5-{(R)-3-[1-(3- isopropyl- [1,2,4]oxadiazol-5- yl)piperidin-4- yl]butoxy}pyrimidin-2- yl)pyrrolidin-3-yl]-5- methylpiperidin-2-one | RT = 2.76 min; m/z (ES⁺) = 541.4 [M + H]⁺ |
| 13 | 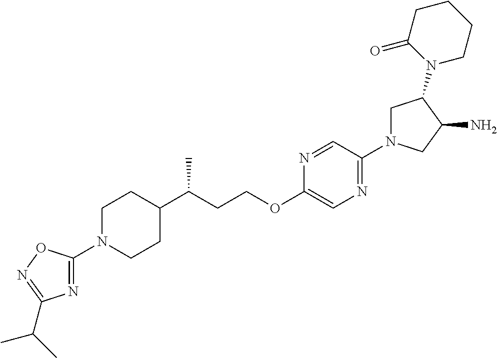 | 1-[3S,4S)-4-Amino-1-(5- {(R)-3-[1-(3-isopropyl- [1,2,4]oxadiazol-5- yl)piperidin-4- yl]butoxy}pyrazin-2- yl)pyrrolidin-3- yl]piperidin-2-one | RT = 2.88 min; m/z (ES⁺) = 527.4 [M + H]⁺ |

### Example 14

### (3R,4S)-4-(2,5-Difluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine p-toluenesulfonic acid salt

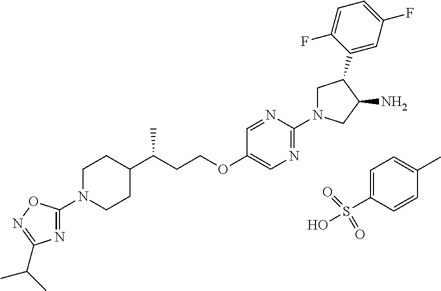

To a solution of [(trans)-4-(2,5-difluorophenyl)-1-(5-{(R)-3-[1-(3-isopropyl-[1,2,4]oxadiazol-5-yl)piperidin-4-yl]butoxy}pyrimidin-2-yl)pyrrolidin-3-yl]carbamic acid tert-butyl ester (Preparation 77, 400 mg, 0.53 mmol) in DCM (5 mL) under argon, cooled to 0° C., was added TFA (1.0 mL) and the reaction was stirred for 1 h before being allowed to reach r.t. A further portion of TFA was added (0.5 mL) and the reaction continued to stir for 2 h before being partitioned between DCM and sat. NaHCO₃ solution. The organic phase was separated, dried (Na₂SO₄) and the solvent removed in vacuo. Purification by chiral HPLC (MTBE:EtOH:BA 80:20:0.1, 11 ml/min, 250 nm, RT=38.0 min) afforded the title compound as its free amine. The product was dissolved in MeOH and TsOH (1eq) was added. Removal of the solvent in vacuo afforded the title compound: RT=3.00 min; m/z (ES⁺)=542.3 [M+H]⁺.

### Example 15

### 4-((R)-3-{2-[(3S,4S)-3-Amino-4-(2-oxopiperidin-1-yl)pyrrolidin-1-yl]pyrimidin-5-yloxy}-1-methylpropyl)piperidine-1-carboxylic acid isopropyl ester p-toluenesulfonic acid salt

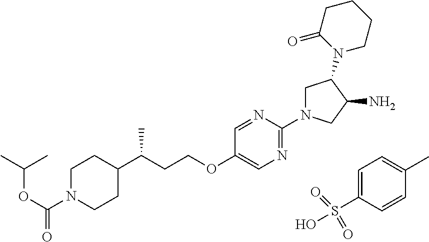

To a solution of 4-((R)-3-{2-[(3S,4S)-3-tert-butoxycarbonylamino-4-(2-oxopiperidin-1-yl)pyrrolidin-1-yl]pyrimidin-5-yloxy}-1-methylpropyl)piperidine-1-carboxylic acid isopropyl ester (Preparation 78, 90 mg, 0.15 mmol) in DCM (5 mL) under argon, cooled to 0° C., was added TFA (1.0 mL). The ice bath was removed and the mixture allowed to stir for 2 h. The reaction was quenched with sat. NaHCO₃ solution, then the organic phase was separated, dried (Na₂SO₄), and the solvent removed in vacuo. Purification by column chromatography (DCM:MeOH, 100:0, 90:10) afforded the title compound as the free amine. To a solution of the product in MeOH was added TsOH (1eq.). Removal of the solvent in vacuo afforded the title compound: RT=2.80 min; m/z (ES⁺)=503.3 [M+H]⁺.

### Example 16

### 1-[(3S,4S)-4-Amino-1-(5-{(R)-3-[1-(3-isopropyl-[1,2,4]oxadiazol-5-yl)piperidin-4-yl]butoxy}-3-methylpyridin-2-yl)pyrrolidin-3-yl]piperidin-2-one p-toluenesulfonic acid salt

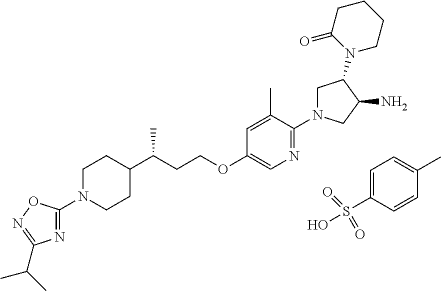

To a solution of [(3S,4S)-1-(5-{(R)-3-[1-(3-isopropyl-[1,2,4]oxadiazol-5-yl)piperidin-4-yl]butoxy}-3-methylpyridin-2-yl)-4-(2-oxopiperidin-1-yl)pyrrolidin-3-yl]carbamic acid tert-butyl ester (Preparation 79, 80 mg, 0.13 mmol) in DCM (5 mL) under argon, cooled to 0° C., was added TFA (2.0 mL) and the resulting solution was stirred for 1 h. The reaction was quenched by the addition of sat. NaHCO₃ solution, then the resulting mixture extracted with DCM. The organic phase was dried (Na₂SO₄) and solvent removed in vacuo. Purification by column chromatography (DCM:MeOH, 100:0, 99:1, 98:2, 97:3, 96:4, 95:5, 90:10) afforded the title compound as the free amine. To a solution of the product in MeOH (2 mL) was added TsOH (1eq.) and the solvent was removed in vacuo. Further purification by preparative HPLC afforded the title compound: RT=2.82 min; m/z (ES⁺)=540.5 [M+H]⁺.

### Example 17

### 4-((R)-3-{2-[(3R,4S)-3-Amino-4-(2,5-difluorophenyl)pyrrolidin-1-yl]pyrimidin-5-yloxy}-1-methylpropyl)piperidine-1-carboxylic acid isopropyl ester p-toluenesulfonic acid salt

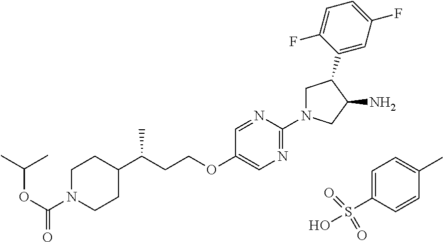

The title compound was prepared from 4-((R)-3-{2-[(3R,4S)-3-tert-butoxycarbonylamino-4-(2,5-difluorophenyl)pyrrolidin-1-yl]pyrimidin-5-yloxy}-1-methylpropyl)piperidine-1-carboxylic acid isopropyl ester (Preparation 80) employing the procedure outlined in Example 16: RT=3.08 min; m/z (ES⁺)=518.5 [M+H]⁺.

### Example 18

### 4-((R)-3-{2-[(3R,4S)-3-Amino-4-(2,4-difluorophenyl)pyrrolidin-1-yl]pyrimidin-5-yloxy}-1-methylpropyl)piperidine-1-carboxylic acid isopropyl ester p-toluenesulfonic acid salt

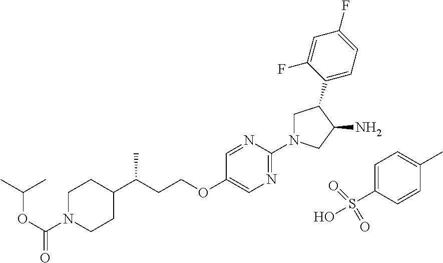

To a solution of 4-((R)-3-{2-[(trans)-3-tert-butoxycarbonylamino-4-(2,4-difluorophenyl)pyrrolidin-1-yl]pyrimidin-5-yloxy}-1-methylpropyl)piperidine-1-carboxylic acid isopropyl ester (Preparation 81, 330 mg, 0.53 mmol) in DCM (5 mL) under argon, cooled to 0° C., was added TFA (2.0 mL) and the resulting solution was stirred for 2 h. The reaction was quenched by the addition of sat. NaHCO₃ solution, then the resulting mixture was extracted with DCM. The organic phase was passed through a phase separator and the solvent removed in vacuo. Purification by column chromatography (DCM:MeOH, 100:0, 98:2, 97:3, 95:5, 90:10) followed by further purification by chiral HPLC (MTBE:EtOH:THF:BA 77:20:3:0.1, 11 mL/min, 250 nm, RT=37.9 min) afforded the title compound as the free amine. To a solution of the product in MeOH was added a solution of TsOH (1eq.) in MeOH. Removal of the solvent in vacuo afforded the title compound: RT=3.10 min; m/z (ES⁺)=518.3 [M+H]⁺.

### Example 19

### 4-((R)-3-{2-[(3S,4R)-3-Amino-4-(2,4-difluorophenyl)pyrrolidin-1-yl]pyrimidin-5-yloxy}-1-methylpropyl)piperidine-1-carboxylic acid isopropyl ester p-toluenesulfonic acid salt

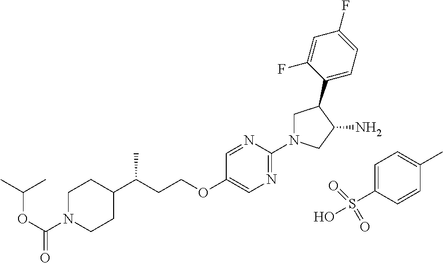

The title compound was prepared from 4-((R)-3-{2-[(trans)-3-tert-butoxycarbonylamino-4-(2,4-difluorophenyl)pyrrolidin-1-yl]pyrimidin-5-yloxy}-1-methylpropyl)piperidine-1-carboxylic acid isopropyl ester (Preparation 81) employing the procedure outlined in Example 18. Chiral HPLC: MTBE:EtOH:THF:BA 77:20:3:0.1, 11 mL/min, 250 nm, RT: 34.0 min. LCMS: RT=3.10 min; m/z (ES⁺)=518.3 [M+H]⁺.

### Example 20

### [(3R,4S)-1-(5-{(R)-3-[1-(5-Chloropyrimidin-2-yl)piperidin-4-yl]butoxy}pyrimidin-2-yl)-4-(2,5-difluorophenyl)pyrrolidin-3-ylamine p-toluenesulfonic acid salt

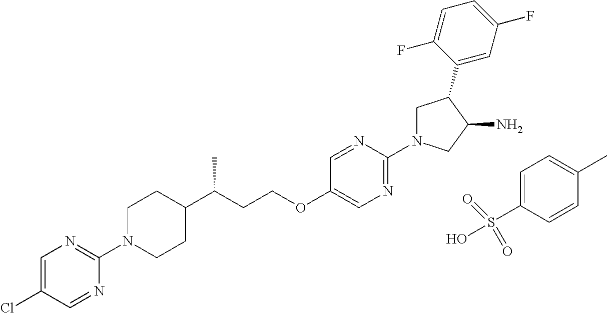

To a solution of [(trans)-1-(5{(R)-3-[1-(5-chloropyrimidin-2-yl)piperidin-4-yl]butoxy}pyrimidin-2-yl)-4-(2,5-difluorophenyl)pyrrolidin-3-yl]carbamic acid tert-butyl ester (Preparation 82, 180 mg, 0.28 mmol) in DCM (4.0 mL) under argon, cooled to 0° C., was added TFA (1.0 mL) and the reaction stirred for 2 h. A further portion of TFA (0.6 mL) was added and stirring continued for 30 min. A further portion of TFA (0.6 mL) was added and stirring continued for 30 min, before allowing the reaction to reach r.t. The reaction was quenched with sat. NaHCO₃ solution, dried (Na₂SO₄) and the solvent removed in vacuo. Purification by chiral HPLC (MTBE:EtOH:THF:BA 60:20:20:0.1, 9 ml/min, 250 nm, RT=14.2 min) afforded the title compound as the free amine. To a solution of the product in MeOH was added a solution of TsOH (1eq) in MeOH. The solvent was removed in vacuo to afford the title compound: RT=3.23 min; m/z (ES⁺)=544.2 [M+H]⁺.

### Example 21

### (3R,4S)-4-(2,5-Difluorophenyl)-1-(6-{(R)-3-[1-(3-isopropyl-[1,2,4]oxadiazol-5-yl)piperidin-4-yl]butoxy}pyrimidin-4-yl)pyrrolidin-3-ylamine

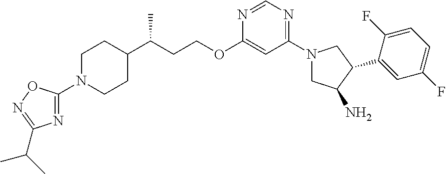

To a solution of [(3R,4S)-4-(2,5-difluorophenyl)-1-(6-{(R)-3-[1-(3-isopropyl-[1,2,4]oxadiazol-5-yl)piperidin-4-yl]butoxy}pyrimidin-4-yl)pyrrolidin-3-yl]carbamic acid tert-butyl ester (Preparation 83, 92 mg, 0.14 mmol) in DCM (2 mL), under argon, was added TFA (0.5 mL) and the reaction was stirred until complete. The mixture was purified by SCX cartridge, eluting with MeOH followed by NH₄OH in MeOH. The basic fraction was concentrated in vacuo to afford the title compound: RT=2.88 min; m/z (ES⁺)=542.3 [M+H]⁺.

### Example 22

### 1-[(3S,4S)-4-Amino-1-(5{(R)-3-[1-(3-isopropyl-[1,2,4]oxadiazol-5-yl)piperidin-4-yl]butoxy}pyrimidin-2-yl)pyrrolidin-3-yl]-(S)-4-methylpiperidin-2-one

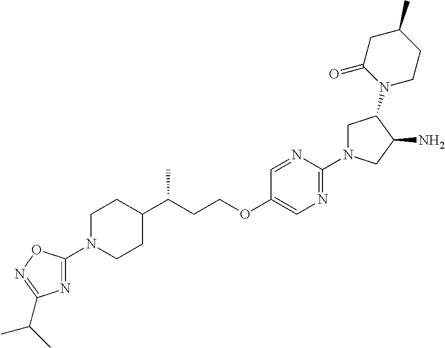

To a solution of [(3S,4S)-1-[5-{(R)-3-[1-(3-isopropyl-[1,2,4]oxadiazol-5-yl)piperidin-4-yl]butoxy}pyrimidin-2-yl)-4-((S)-4-methyl-2-oxopiperidin-1-yl)pyrrolidin-3-yl]carbamic acid tert-butyl ester (Preparation 86, 47 mg, 0.07 mmol) in DCM (5.0 mL), under argon, cooled to 0° C., was added TFA (1.3 mL) and the mixture was stirred at this temperature until complete. The reaction was quenched with sat. Na₂CO₃ solution (30 mL) and the mixture extracted with EtOAc (2×30 mL). The organic phase was washed with brine (30 mL), then dried (MgSO₄). Removal of the solvent in vacuo and purification by column chromatography (DCM:MeOH:NH₄OH, 97:3:0.3) afforded the title compound: RT=2.74 min; m/z (ES⁺)=541.3 [M+H]⁺.

### Example 23

### 1-[(3S,4S)-4-Amino-1-(5{(R)-3-[1-(3-isopropyl-[1,2,4]oxadiazol-5-yl)piperidin-4-yl]butoxy}pyrimidin-2-yl)pyrrolidin-3-yl]-(R)-4-methylpiperidin-2-one p-toluenesulfonic acid salt

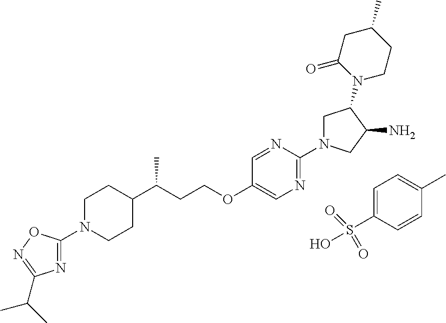

To a solution of [(3S,4S)-1-[5-{(R)-3-[1-(3-isopropyl-[1,2,4]oxadiazol-5-yl)piperidin-4-yl]butoxy}pyrimidin-2-yl)-4-((R)-4-methyl-2-oxopiperidin-1-yl)pyrrolidin-3-yl]carbamic acid tert-butyl ester (Preparation 87, 52 mg, 0.08 mmol) in DCM (5.0 mL), under argon, cooled to 0° C., was added TFA (1.3 mL) and the reaction was stirred at this temperature for 4 h. The reaction was quenched with sat. Na₂CO₃ solution (30 mL) and the mixture extracted with EtOAc (2×30 mL). The organic phase was washed with brine (30 mL), then dried (MgSO₄). Removal of the solvent in vacuo and purification by column chromatography (DCM:MeOH:NH₄OH, 97:3:0.3) afforded the title compound as the free amine. To a solution of the product in DCM (2 mL) was added a solution of TsOH (1eq.) in MeOH (2 mL). The mixture was stirred then removal of the solvent in vacuo afforded the title compound: RT=2.86 min; m/z (ES⁺)=541.3 [M+H]⁺.

### Example 24

### (3′S,4′S)-4′-Amino-1′-(5-{(R)-3-[1-(3-isopropyl-[1,2,4]oxadiazol-5-yl)piperidin-4-yl]butoxy}pyrimidin-2-yl)-[1,3]bipyrrolidinyl-2-one p-toluenesulfonic acid salt

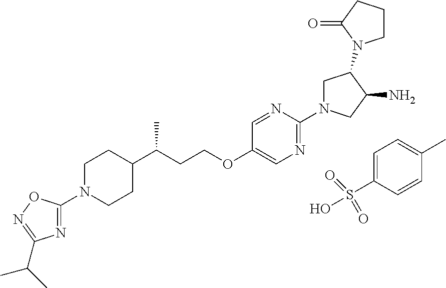

To a solution of [(3′S,4′S)-1′-(5-{(R)-3-[1-(3-isopropyl-[1,2,4]oxadiazol-5-yl)piperidin-4-yl]butoxy}pyrimidin-2-yl)-2-oxo-[1,3′]bipyrrolidinyl-4′-yl]carbamic acid tert-butyl ester (Preparation 88, 105 mg, 0.17 mmol) in dioxane (3 mL), under argon, was added a solution of HCl in dioxane (4M, 1.5 mL) and the mixture was stirred at r.t. for 16 h. The reaction solvent was concentrated in vacuo and Et₂O was added. The mixture was stirred for 2 min then the Et₂O decanted. The crude material was partitioned between EtOAc (20 mL) and sat. Na₂CO₃ solution (20 mL). The organic phase was separated, washed with brine (30 mL) then dried (MgSO₄). Removal of the solvent in vacuo and purification by column chromatography (DCM:MeOH:NH₄OH, 97:3:0.3) afforded the title compound as the free amine. To a solution of the product in DCM (2 mL) was added a solution of TsOH (1eq.) in MeOH (2 mL). The solution was stirred, then the solvent was removed in vacuo to afford the title compound: RT=2.69 min; m/z (ES⁺)=513.3 [M+H]⁺.

### Example 25

### 1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]-5,5-difluoropiperidin-2-one p-toluenesulfonic acid salt

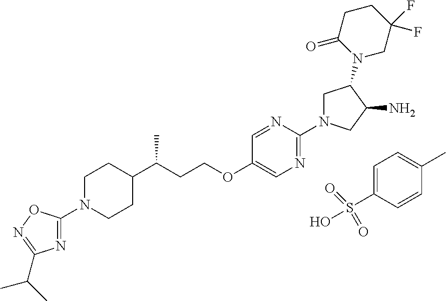

To a solution of [(3S,4S)-4-(5,5-difluoro-2-oxopiperidin-1-yl)-1-(5-{(R-3[1-(3-isopropyl[1,2,4]oxadiazol-5-yl)piperidin-4-yl]butoxy}pyrimidin-2-yl)pyrrolidin-3-yl]carbamic acid tert-butyl ester (Preparation 89, 143 mg, 0.22 mmol) in DCM (5 mL), under argon, was added TFA (1.0 mL) and the reaction was stirred until complete. The mixture was purified by SCX cartridge, eluting with MeOH followed by NH₄OH in MeOH. The basic fraction was concentrated in vacuo then redissolved in a small volume of MeOH. TsOH (1eq.) was added and the mixture stirred. Removal of the solvent in vacuo afforded the title compound: RT=2.80 min; m/z (ES⁺)=563.3 [M+H]⁺.

The following examples were prepared by reacting the appropriate tert-butyl carbamate protected intermediate with TFA, employing the procedure outlined in Example 25:

| Ex. | Structure | Name | LCMS |
|---|---|---|---|
| 26 | 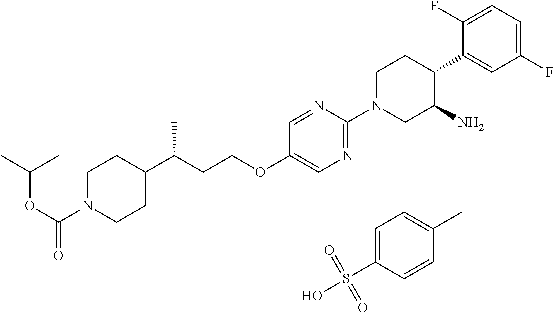 | 4-((R)-3-{2-[(3R,4R)-3- Amino-4-(2,5- difluorophenyl)piperidin- 1-yl]pyrimidin-5-yloxy}- 1-methylpropyl)- piperidine-1-carbocylic acid isopropyl ester p- toluenesulfonic acid salt | RT = 3.10 min m/z (ES⁺) = 532.3 [M + H]⁺ |
| 27 | 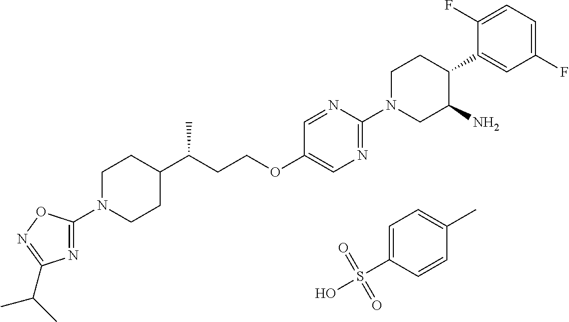 | (3R,4R)-4-(2,5- Difluorophenyl)-1-(5- {(R)-3-[1-(3-isopropyl- [1,2,4]oxadiazol-5- yl)piperidin-4- yl]butoxy}pyrimidin-2- yl)piperidin-3-ylamine p- toluenesulfonic acid salt | RT = 3.07 min m/z (ES⁺) = 556.4 [M + H]⁺ |

### Example 28

### 1-[(3S,4S)-4-Amino-1-(5-{3-[1-(3-isopropyl-[1,2,4]oxadiazol-5-yl)piperidin-4-yl]propoxy}pyrimidin-2-yl)pyrrolidin-3-yl]piperidin-2-one

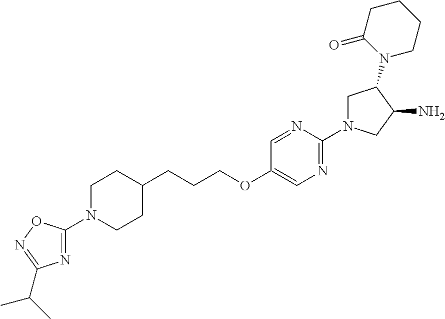

To a solution of [(3S,4S)-1-[5-{3-[1-(3-isopropyl-[1,2,4]oxadiazol-5-yl)piperidin-4-yl]propoxy}pyrimidin-2-yl)-4-(2-oxopiperidin-1-yl)pyrrolidin-3-yl]carbamic acid tert-butyl ester (Preparation 90, 47 mg, 0.07 mmol) in DCM (5 mL) under argon, cooled to 0° C. was added TFA (1 mL) and the reaction was allowed to stir at this temperature for 3 h. A further portion of TFA (0.3 mL) was added and stirring continued until completion. The reaction was quenched with sat. Na₂CO₃ solution (30 mL) then the mixture was extracted with EtOAc (2×30 mL). The organic fractions were combined, washed with brine (30 mL), and dried (MgSO₄). Removal of the solvent in vacuo and purification by column chromatography (DCM:MeOH:NH₄OH, 97:3:0.3) afforded the title compound: RT=2.64 min; m/z (ES⁺)=513.3 [M+H]⁺.

### Example 29

### (3R,4S)-4-(2-Fluorophenyl)-1-(5-{3-[1-(3-isopropyl-[1,2,4]oxadiazol-5-yl)piperidin-4-yl]propoxy}pyrimidin-2-yl)pyrrolidin-3-ylamine p-toluenesulfonic acid salt

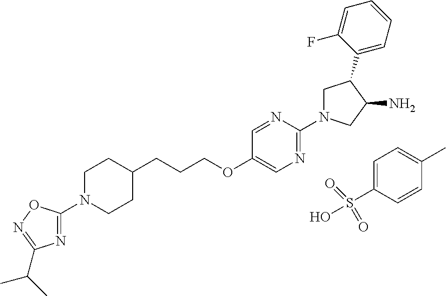

To a solution of 2-chloro-5-{3-[1-(3-isopropyl-[1,2,4]oxadiazol-5-yl)piperidin-4-yl]propoxy}pyrimidine (Preparation 19, 156 mg, 0.43 mmol) and [(trans)-4-(2-fluorophenyl)pyrrolidin-3-yl]carbamic acid-9H-fluoren-9-ylmethyl ester hydrochloride (Preparation 98, 227 mg, 0.52 mmol) in DMSO (0.9 mL), under argon, was added DBU (130 μL, 0.86 mmol) and the mixture was heated to 85° C. until no further reaction was observed. Water (10 mL) was added and the reaction mixture extracted with EtOAc (2×20 mL). The organic fractions were combined, washed with water (20 mL), sat. Na₂CO₃ solution, brine (40 mL), then dried (MgSO₄), and the solvent was removed in vacuo. Purification by column chromatography (DCM:MeOH:NH₄OH, 98:2:0.1) followed by chiral HPLC (MTBE:MeOH:BA 80:20:0.1, 12 ml/min, 250 nm, RT=35.2 min) afforded the title compound as the free amine. To a solution of the product in DCM was added TsOH (1eq.) in MeOH. The mixture was stirred then removal of the solvent in vacuo afforded the title compound: RT=2.93 min; m/z (ES⁺)=510.3 [M+H]⁺.

### Example 30

### (3R,4S)-4-(2-Fluorophenyl)-1-(5-{(R)-3-[1-[3-isopropyl-[1,2,4]oxadiazol-5-yl)piperidin-4-yl]butoxy}-4-methylpyrimidin-2-yl)pyrrolidin-3-ylamine p-toluenesulfonic acid salt

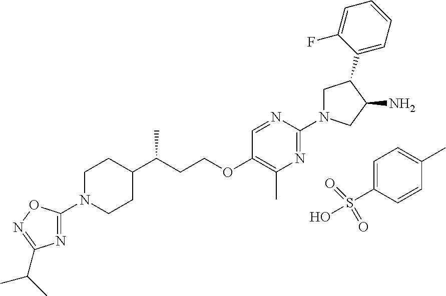

To a solution of (R)-2-chloro-5-{3-[1-(3-isopropyl-[1,2,4]oxadiazol-5-yl)piperidin-4-yl]butoxy}-4-methylpyrimidine (Preparation 10, 150 mg, 0.38 mmol) and [trans-4-(2-fluorophenyl)pyrrolidin-3-yl]carbamic acid-9H-fluoren-9-ylmethyl ester hydrochloride (Preparation 98, 184 mg, 0.42 mmol) in DMSO (0.75 mL), under argon, was added DBU (114 μL, 0.76 mmol) and the reaction was heated to 85° C. in a sealed tube for 24 h. The mixture was partitioned between EtOAc (30 mL) and water (30 mL), and the organic phase was separated. The aqueous phase was extracted with EtOAc (3×30 mL), then the organic fractions were combined, washed with water (2×20 mL), sat. Na₂CO₃ solution (50 mL), brine (2×50 mL), and dried (MgSO₄). Removal of the solvent in vacuo and purification by column chromatography (DCM:MeOH:NH₄OH, 97:3:0.3) followed by chiral HPLC (MTBE:EtOH:BA 80:20:0.1, 11 ml/min, 250 nm, RT=17.0 min) afforded the title compound as the free amine. To a solution of the product in DCM was added TsOH (1eq.) in MeOH. The mixture was stirred then removal of the solvent in vacuo afforded the title compound: RT=3.04 min; m/z (ES⁺)=538.3 [M+H]⁺.

### Example 31

### 4-((R)-3-{2-[(3R,4S)-3-Amino-4-(2-fluorophenyl)pyrrolidin-1-yl]pyrimidin-5-yloxy}-1-methylpropyl)piperidine-1-carboxylic acid isopropyl ester p-toluenesulfonic acid salt

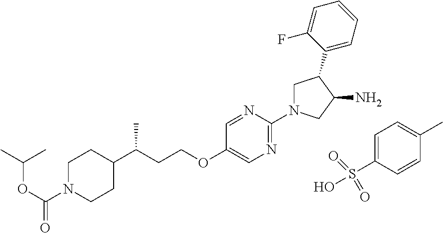

The title compound was prepared by reacting 4-[(R)-3-(2-chloropyrimidin-5-yloxy)-1-methylpropyl]piperidine-1-carboxylic acid isopropyl ester (Preparation 17) with [trans-4-(2-fluorophenyl)pyrrolidin-3-yl]carbamic acid-9H-fluoren-9-ylmethyl ester hydrochloride (Preparation 98) employing the procedure outlined in Example 29. Chiral HPLC: MTBE:MeOH:THF:BA 70:20:10:0.1, 11 ml/min, 250 nm, RT=15.4 min. LCMS: RT=2.87 min; m/z (ES⁺)=500.3 [M+H[⁺.

### Example 32

### (3R,4S)-1-(5-{(R)-3-[1-(3-Ethyl-[1,2,4]oxadiazol-5-yl)piperidin-4-yl]butoxy}pyrimidin-2-yl)-4-(2-fluorophenyl)pyrrolidin-3-ylamine p-toluenesulfonic acid salt

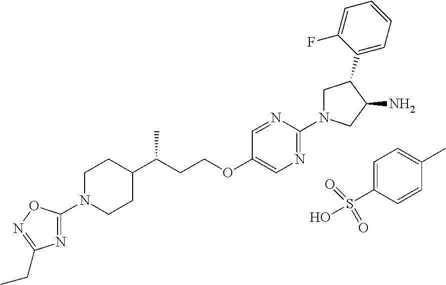

To a solution of 2-chloro-5-{(R)-3-[1-(3-ethyl-[1,2,4]oxadiazol-5-yl)piperidin-4-yl]butoxy}pyrimidine (Preparation 21, 150 mg, 0.41 mmol) and [trans-4-(2-fluorophenyl)pyrrolidin-3-yl]carbamic acid-9H-fluoren-9-ylmethyl ester hydrochloride (Preparation 98, 216 mg, 0.49 mmol) in DMSO (0.8 mL), under argon, was added DBU (120 μL, 0.82 mmol) and the reaction was heated to 80° C. for 16 h. Water (15 mL) was added and the mixture was extracted with EtOAc (4×20 mL). The organic fractions were combined, washed with water (2×20 mL), brine (70 mL) and dried (MgSO₄) before removal of the solvent in vacuo. The crude residue was passed down an SCX cartridge, eluting with MeOH then NH₄OH in MeOH, and the basic fraction collected. Purification by column chromatography (DCM:MeOH, 99:1, 98:2, 97:3) followed by chiral HPLC (MTBE:MeOH:BA 80:20:0.1, 12 ml/min, 250 nm, RT=40.6 min) afforded the title compound as the free amine. To a solution of the product in MeOH was added TsOH (1eq.). The solvent was removed in vacuo to afford the title compound: RT=2.79 min; m/z (ES⁺)=510.3 [M+H]⁺.

### Example 33

### (3R,4S)-1-(5-{(R)-3-[1-(3-Isopropyl-[1,2,4]oxadiazol-5-yl)piperidin-4-yl]butoxy}pyrimidin-2-yl)-4-(2,4,5-trifluorophenyl)pyrrolidin-3-ylamine p-toluenesulfonic acid salt

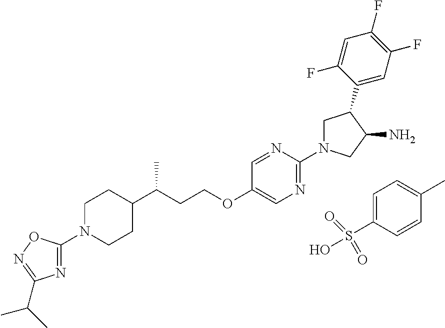

[(3R,4S)-1-(5-{(R)-3-[1-(3-isopropyl-[1,2,4]oxadiazol-5-yl)piperidin-4-yl]butoxy}pyrimidin-2-yl)-4-(2,4,5-trifluorophenyl)pyrrolidin-3-yl]carbamic acid tert-butyl ester (Preparation 91) was reacted employing the procedure outlined in Example 21. Further purification by column chromatography (DCM:MeOH:NH₄OH, 98:2:0.1) afforded the title compound as the free amine. To a solution of the product in MeOH was added TsOH (1eq.). The solvent was removed in vacuo to afford the title compound: RT=3.01 min; m/z (ES⁺)=560.5 [M+H]⁺.

### Example 34

### (3R,4S)-1-(5-{(R)-3-[1-(3-Ethyl-[1,2,4]oxadiazol-5-yl)piperidin-4-yl]butoxy}pyrimidin-2-yl)-4-(2,4,5-trifluorophenyl)pyrrolidin-3-ylamine p-toluenesulfonic acid salt

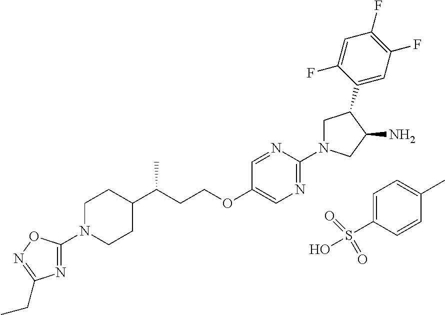

The title compound was prepared from [(3R,4S)-1-(5-{(R)-3-[1-(3-ethyl-[1,2,4]oxadiazol-5-yl)piperidin-4-yl]butoxy}pyrimidin-2-yl)-4-(2,4,5-trifluorophenyl)pyrrolidin-3-yl]carbamic acid tert-butyl ester (Preparation 92) employing the procedure outlined in Example 33: RT=2.86 min; m/z (ES⁺)=546.5 [M+H]⁺.

### Example 35

### (3R,4S)-4-(2,5-Difluorophenyl)-1-(5-{(R)-3-[1-(3-isopropyl-[1,2,4]oxadiazol-5-yl)piperidin-4-yl]butoxy}-3-methylpyridin-2-yl)pyrrolidin-3-ylamine p-toluenesulfonic acid salt

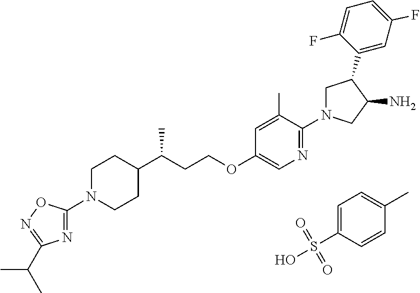

The title compound was prepared from [(3R,4S)-4-(2,5-difluorophenyl)-1-(5-{(R)-3-[1-(3-isopropyl[1,2,4]oxadiazol-5-yl)piperidin-4-yl]butoxy}-3-methylpyridin-2-yl)pyrrolidin-3-yl]carbamic acid tert-butyl ester (Preparation 93) employing the procedure outlined in Example 18. After work-up, purification of the crude material by preparative HPLC afforded the title compound as the free amine. To a solution of the product in MeOH was added TsOH (1eq.). The solution was stirred then the solvent was removed in vacuo to afford the title compound: RT=3.05 min; m/z (ES⁺)=555.3 [M+H]⁺.

### Example 36

### (3R,4S)-1-(5-{(R)-3-[1-(5-Chloropyrimidin-2-yl)piperidin-4-yl]butoxy}pyrimidin-2-yl)-4-(2-fluorophenyl)pyrrolidin-3-ylamine p-toluenesulfonic acid salt

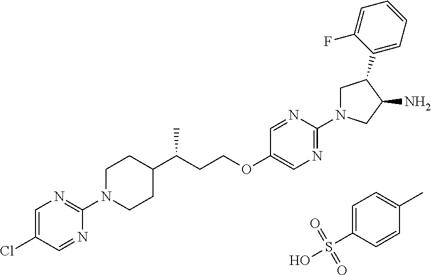

To a solution of (R)-5-chloro-2-{4-[1-methyl-3-(2-chloropyrimidin-5-yloxy)propyl]-piperidin-1-yl}pyrimidine (Preparation 4, 175 mg, 0.46 mmol) and [trans-4-(2-fluorophenyl)pyrrolidin-3-yl]carbamic acid-9H-fluoren-9-ylmethyl ester hydrochloride (Preparation 98, 201 mg, 0.46 mmol) in DMSO (2.5 mL), under argon, was added DBU (240 μL, 1.60 mmol). The mixture was heated to 100° C. in a sealed tube until no further reaction was observed. The reaction mixture was diluted with EtOAc, washed with brine, sat. NaHCO₃ solution, and dried (Na₂SO₄) before removal of the solvent in vacuo. Purification by column chromatography followed by chiral HPLC (MTBE:EtOH:THF:BA 70:20:10:0.1, 11 ml/min, 250 nm, RT=19.2 min) afforded the title compound as the free amine. To a solution of the product in MeOH was added TsOH (1eq.). The solvent was removed in vacuo to afford the title compound: RT=3.34 min; m/z (ES⁺)=526.2 [M+H]⁺.

### Example 37

### (3R,4R)-4-(2,5-Difluorophenyl)-5′-{(R)-3-[1-(3-isopropyl-[1,2,4]oxadiazol-5-yl)piperidin-4-yl]butoxy}-3,4,5,6-tetrahydro-2H-[1,2′]bipyridinyl-3-ylamine dihydrochloride

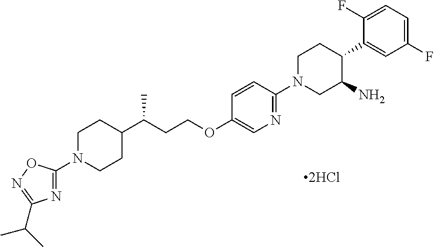

To a solution of (R)-2-bromo-5-{3-[1-(3-isopropyl-[1,2,4]oxadiazol-5-yl)piperidin-4-yl]butoxy}pyridine (Preparation 5, 127 mg, 0.30 mmol) in dioxane (4 mL) was added [(3R,4R)-4-(2,5-difluorophenyl)piperidin-3-yl]carbamic acid tert-butyl ester (Preparation 50, 112 mg, 0.36 mmol), 2,8,9-triisobutyl-2,5,8,9-tetraaza-1-phosphabicyclo-[3.3.3]undecane (10 mg, 0.03 mmol) and sodium tert-butoxide (101 mg, 1.05 mmol), and the mixture was bubbled with argon for 20 min. tris-(Dibenzylideneacetone)-dipalladium (27 mg, 0.03 mmol) was added and the mixture bubbled with argon for a further 10 min before being heated in a microwave reactor at 120° C. until complete. The crude mixture was passed down an SCX cartridge, eluting with MeOH followed by NH₄OH in MeOH. The basic fraction was concentrated in vacuo. Further purification by preparative HPLC afforded the title compound as the free amine. The product was re-dissolved in a solution of HCl in dioxane (4M) and the solvent was removed in vacuo to afford the title compound: RT=3.04 min; m/z (ES⁺)=555.5 [M+H]⁺.

### Example 38

### (3R,4R)-4-(2,5-Difluorophenyl)-1-(5-{(R)-3-[1-(3-isopropyl-[1,2,4]oxadiazol-5-yl)piperidin-4-yl]butoxy}pyrazin-2-yl)piperidin-3-ylamine hydrochloride

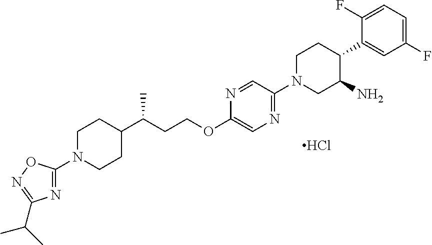

The title compound was prepared by reacting (R)-2-Bromo-5-{3-[1-(3-isopropyl-[1,2,4]oxadiazol-5-yl)piperidin-4-yl]butoxy}pyrazine (Preparation 13) with [(3R,4R)-4-(2,5-difluorophenyl)piperidin-3-yl]carbamic acid tert-butyl ester (Preparation 50) employing the procedure outlined in Example 37: RT=3.26 min; m/z (ES⁺)=556.2 [M+H]⁺.

### Example 39

### (3R,4R)-4-(2,5-Difluorophenyl)-5′-{3-[1-(3-isopropyl-[1,2,4]oxadiazol-5-yl)piperidin-4-yl]propoxy}-3,4,5,6-tetrahydro-2H-[1,2′]bipyridinyl-3-ylamine dihydrochloride

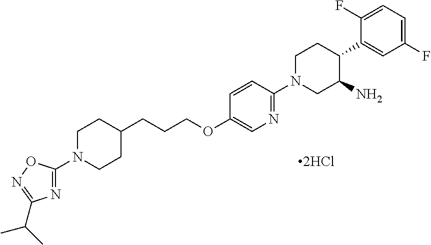

A combination of 2-bromo-5-{3-[1-(3-isopropyl-[1,2,4]oxadiazol-5-yl)piperidin-4-yl]propoxy}pyridine (123 mg, 0.29 mmol), [(3R,4R)-4-(2,5-difluorophenyl)piperidin-3-yl]carbamic acid tert-butyl ester (Preparation 50, 109 mg, 0.35 mmol), 9,9-dimethyl-4,5-bis(diphenylphosphino)xanthene (10 mg, 0.02 mmol) and sodium tert-butoxide (101 mg, 1.05 mmol), in toluene (4 mL), was bubbled with argon for 15 min. tris-(Dibenzylideneacetone)-dipalladium (5 mg, 0.01 mmol) was added and the resulting mixture was heated to 90° C. for 24 h. The crude mixture was filtered and purified by SCX cartridge, eluting with MeOH followed by NH₄OH in MeOH. The basic fraction was concentrated in vacuo. The resulting residue was dissolved in DCM, and TFA was added before stirring the mixture for 1 h. The reaction mixture was purified by SCX cartridge, eluting with MeOH followed by NH₄OH in MeOH. Further purification by preparative HPLC afforded the title compound as the free amine. The product was re-dissolved in a solution of HCl in dioxane (4M) and solvent was removed in vacuo to afford the title compound: RT=2.88 min; m/z (ES⁺)=541.4 [M+H]⁺.

### Example 40

### (3R,4R)-4-(2,5-Difluorophenyl)-1-(5-{3-[1-(3-isopropyl-[1,2,4]oxadiazol-5-yl)piperidin-4-yl]propoxy}pyrimidin-2-yl)piperidin-3-ylamine p-toluenesulfonic acid salt

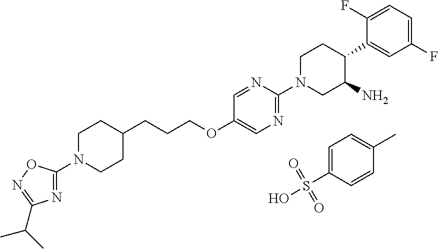

To a solution of 2-chloro-5-{3-[1-(3-isopropyl-[1,2,4]oxadiazol-5-yl)piperidin-4-yl]propoxy}pyrimidine (Preparation 19, 197 mg, 0.50 mmol) and [(3R,4R)-4-(2,5-difluorophenyl)piperidin-3-yl]carbamic acid tert-butyl ester (Preparation 50, 156 mg, 0.50 mmol) in DMSO (1.0 mL) was added DBU (75 μL, 0.5 mmol) and the mixture was heated to 100° C. for 40 h. The crude mixture was diluted with DCM and the solution washed with brine, passed through a phase separater and concentrated in vacuo. The crude product was re-dissolved in MeOH and passed down an SCX cartridge, eluting with MeOH then NH₄OH in MeOH. The basic fraction was collected and concentrated in vacuo, then triturated with MeOH to afford [(3R,4R)-4-(2,5-difluorophenyl)-1-(5-{3-[1-(3-isopropyl-[1,2,4]oxadiazol-5-yl)piperidin-4-yl]propoxy}pyrimidin-2-yl)piperidin-3-yl]carbamic acid tert-butyl ester: RT=4.89 min; m/z (ES⁺)=642.3 [M+H]⁺.

To a solution of the material in DCM (10 mL) was added TFA (4 mL) and the reaction was stirred at r.t. for 30 min. The crude mixture was passed down an SCX cartridge, eluting with MeOH then NH₄OH in MeOH. The basic fraction was collected, concentrated in vacuo, and purified further by preparative HPLC to afford the title compound as the free amine. A solution of TsOH (1eq.) in MeOH was added to the product, then the solvent removed in vacuo to afford the title compound: RT=3.02 min; m/z (ES⁺)=542.3 [M+H]⁺.

### Example 41

### (3R,4R)-1-(5-{3-[1-(5-Chloropyrimidin-2-yl)piperidin-4-yl]propoxy}pyrimidin-2-yl)-4-(2,5-difluorophenyl)piperidin-3-ylamine p-toluenesulfonic acid salt

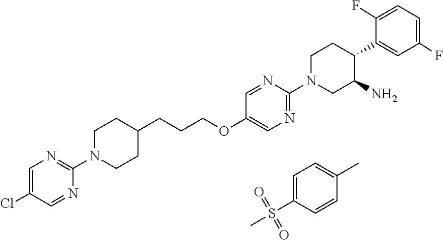

The title compound was prepared by reacting 5-chloro-2-{4-[5-(2-chloropyrimidin-5-yloxy)propyl]piperidin-1-yl}pyrimidine (Preparation 23) with [(3R,4R)-4-(2,5-difluorophenyl)piperidin-3-yl]carbamic acid tert-butyl ester (Preparation 50) employing the procedure outlined in Example 40, although without the need for further purification by preparative HPLC: RT=3.25 min; m/z (ES⁺)=544.3 [M+H]⁺.

### Example 42

### (3R,4R)-1-(5-{3-[1-(5-Chloropyrimidin-2-yl)piperidin-4-yl]propoxy}pyrimidin-2-yl)-4-(2-fluorophenyl)piperidin-3-ylamine

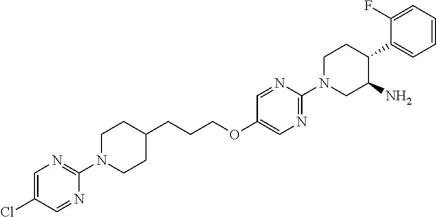

To a mixture of 5-chloro-2-{4-[5-(2-chloropyrimidin-5-yloxy)propyl]piperidin-1-yl}pyrimidine (Preparation 23, 162 mg, 0.55 mmol) and [(3R,4R)-4-(2-fluorophenyl)piperidin-3-yl]carbamic acid tert-butyl ester (Preparation 55, 130 mg, 0.33 mmol) in DMSO (2 mL) was added DBU (80 μL, 0.55 mmol) and the mixture was heated to 80° C. until complete. The crude mixture was partitioned between EtOAc and water, then the organic phase was separated, washed with brine, dried (MgSO₄), and the solvent was removed in vacuo. Purification by column chromatography (IH:EtOAc, 3:1) afforded the intermediate product [(3R,4R)-1-(5-{3-[1-(5-chloropyrimidin-2-yl)piperidin-4-yl]propoxy}pyrimidin-2-yl)-4-(2-fluorophenyl)piperidin-3-yl]carbamic acid tert-butyl ester: RT=5.28 min; m/z (ES⁺)=626.4 [M +H]⁺. To a solution of the product in DCM (10 mL) was added TFA (2.5 mL) and the reaction was stirred at r.t. until complete. The reaction solvent was concentrated in vacuo and the resulting residue partitioned between EtOAc (300 mL) and 1M NaOH (100 mL). The organic phase was separated, washed with brine, and dried (MgSO₄). Removal of the solvent in vacuo and purification by column chromatography (DCM:MeOH, 20:1) afforded the title compound: RT=3.32 min; m/z (ES⁺)=526.2 [M+H]⁺.

The following examples were prepared by reaction of the relevant chloropyrimidine with the appropriate amine building block, and subsequent deprotection, employing the procedure outlined in Example 42:

| Ex. | Structure | Name | LCMS |
|---|---|---|---|
| 43 | 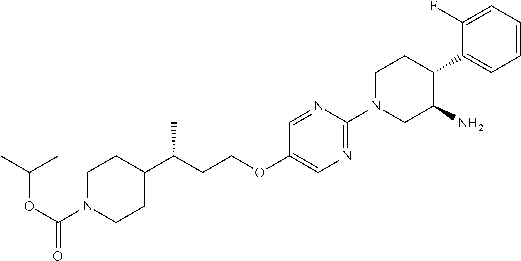 | 4-((R)-3-{2-[(3R,4R)-3- Amino-4-(2- fluorophenyl)piperidin-1- yl]pyrimidin-5-yloxy}-1- methylpropyl)piperidine- 1-carboxylic acid isopropyl ester | RT = 3.15 min m/z (ES⁺) = 514.3 [M + H]⁺ |
| 44 | 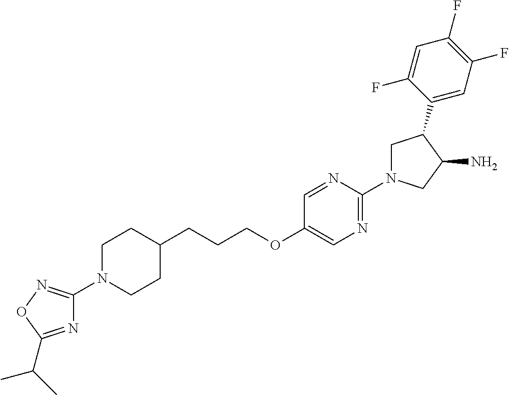 | (3R,4S)-1-(5-{3-[1-(5- isopropyl- [1,2,4]oxadiazol-3- yl)piperidin-4- yl]propoxy}pyrimidin-2- yl)-4-(2,4,5- trifluorophenyl)pyrrolidin- 3-ylamine | RT = 3.21 min m/z (ES⁺) = 546.4 [M + H]⁺ |

### Example 45

### 4-((R)-3-{2-[(3R,4S)-3-Amino-4-(2,4,5-trifluorophenyl)pyrrolidin-1-yl]pyrimidin-5-yloxy}-1-methylpropyl)piperidine-1-carboxylic acid isopropyl ester hydrochloride

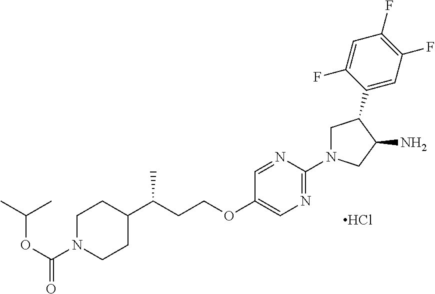

To a mixture of 4-[(R)-3-(2-chloropyrimidin-5-yloxy)-1-methylpropyl[piperidine-1-carboxylic acid isopropyl ester (Preparation 17, 160 mg, 0.51 mmol)) and [(3R,4S)-4-(2,4,5-trifluorophenyl)pyrrolidin-3-yl]carbamic acid tert-butyl ester (Preparation 66, 190 mg, 0.50) in DMSO (2 mL) was added DBU (80 μL, 0.55 mmol) and the reaction was heated to 80° C. until complete. The crude mixture was partitioned between EtOAc and water, then the organic phase was separated, washed with brine, dried (MgSO₄), and the solvent removed in vacuo. Purification by column chromatography (IH:EtOAc, 1.5:1) afforded the intermediate product 4-((R)-3-{2-[(3R,4S)-3-tert-butoxycarbonylamino-4-(2,4,5-trifluorophenyl)pyrrolidin-1-yl]pyrimidin-5-yloxy}-1-methylpropyl)piperidine-1-carboxylic acid isopropyl ester: RT=4.84 min; m/z (ES⁺)=636.4 [M+H]⁺. To a solution of the product in DCM (10 mL) was added TFA (2.5 mL) and the reaction was stirred at r.t. until complete. The reaction solvent was concentrated in vacuo and the resulting residue was partitioned between EtOAc (300 mL) and 1M NaOH (100 mL). The organic phase was separated, washed with brine, then dried (MgSO₄). Removal of the solvent in vacuo and purification by column chromatography (DCM:MeOH, 20:1) afforded the title compound as the free amine. To a solution of the product in MeOH was added 1M HCl solution, and the solvent was removed in vacuo to afford the title compound: RT=3.15 min; m/z (ES⁺)=536.3 [M+H]⁺.

The following Examples were prepared by reaction of the relevant chloropyrimidine with the appropriate amine building block, and subsequent deprotection, employing the procedure outlined in Example 45:

| Ex. | Structure | Name | LCMS |
|---|---|---|---|
| 46 | 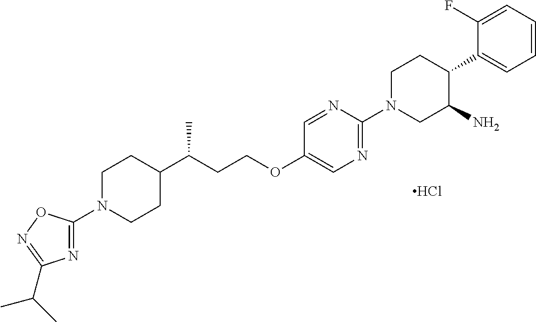 | (3R,4R)-4-(2- Fluorophenyl)-1-(5-{(R)- 3-[1-(3-isopropyl- [1,2,4]oxadiazol-5- yl)piperidin-4- yl]butoxy}pyrimidin-2- yl)piperidin-3-ylamine hydrochloride | RT = 3.08 min m/z (ES⁺) = 538.3 [M + H]⁺ |
| 47 | 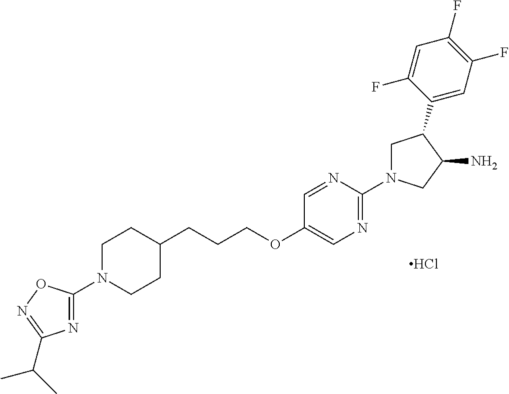 | (3R,4S)-1-(5-{3-[1-(3- Isopropyl- [1,2,4]oxadiazol-5- yl)piperidin-4- yl]propoxy}pyrimidin-2- yl)-4-(2,4,5- trifluorophenyl)pyrrolidin- 3-ylamine | RT = 3.10 min m/z (ES⁺) = 546.4 [M + H]⁺ |

### Example 48

### (3R,4S)-4-(2-Fluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine

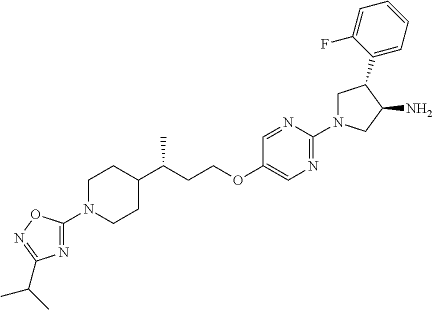

To a solution of (R)-2-chloro-5-{3-[1-(3-isopropyl-[1,2,4]oxadiazol-5-yl)piperidin-4-yl]butoxy}pyrimidine (Preparation 7, 174 mg, 0.46 mmol) and [(trans)-4-(2-fluorophenyl)pyrrolidin-3-yl]carbamic acid 9H-fluoren-9-ylmethyl ester hydrochloride (Preparation 98, 202 mg, 0.46 mmol) in DMSO (0.92 mL) was added DBU (240 μL, 1.60 mmol) and the reaction was heated to 100° C. for 20 h. The mixture was partitioned between EtOAc and water, then the organic phase was separated, washed with water (×5), brine, dried (MgSO₄) and the solvent removed in vacuo. Purification by column chromatography (DCM:MeOH, 95:5, 90:10) and further purification by chiral HPLC (MTBE:MeOH:BA 80:20:0.1, 12 mL/min, 250 nm, RT=28.7 min) afforded the title compound: RT=2.11 min; m/z (ES⁺)=524.5 [M+H]⁺.

### Example 49

### (3S,4R)-4-(2-Fluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine

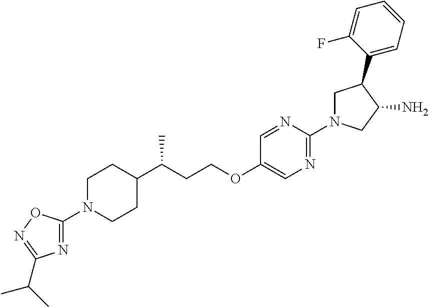

The title compound was prepared from (R)-2-chloro-5-{3-[1-(3-isopropyl-[1,2,4]oxadiazol-5-yl)piperidin-4-yl]butoxy}pyrimidine (Preparation 7) and [(trans)-4-(2-fluorophenyl)pyrrolidin-3-yl]carbamic acid 9H-fluoren-9-ylmethyl ester hydrochloride (Preparation 98) employing the procedure outlined in Example 48. Chiral HPLC: MTBE:MeOH:BA 80:20:0.1, 12 mL/min, 250 nm, RT=34.9 min. LCMS: RT=2.11 min; m/z (ES⁺)=524.5 [M+H]⁺.

### Example 50

### 4-((R)-4-{2-[(3S,4S)-3-Amino-4-(2-oxopiperidin-1-yl)pyrrolidin-1-yl]pyrimidin-5-yl}-1-methylbutyl)piperidine-1-carboxylic acid isopropyl ester

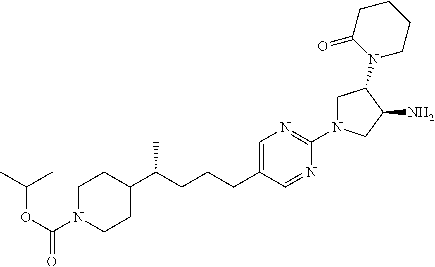

To a solution of 4-((R)-4-{2-[(3S,4S)-3-tert-butoxycarbonylamino-4-(2-oxopiperidin-1-yl)pyrrolidin-1-yl]pyrimidin-5-yl}-1-methylbutyl)piperidine-1-carboxylic acid isopropyl ester (Preparation 105, 17 mg, 0.03 mmol) in DCM (1.0 mL) under argon, cooled to 0° C., was added TFA (0.2 mL) and the reaction was stirred at this temperature until complete. The solvent was removed in vacuo and the resulting residue re-dissolved in MeOH. The solution was purified by SCX cartridge, eluting with MeOH followed by NH₄OH in MeOH. The basic fraction was concentrated in vacuo, and further purification by column chromatography (DCM:MeOH, 95:5) afforded the title compound: RT=2.85 min; m/z (ES⁺)=501.5 [M+H]⁺.

### Example 51

### (3R,4S)-4-(2,4-Difluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine p-toluenesulfonic acid salt

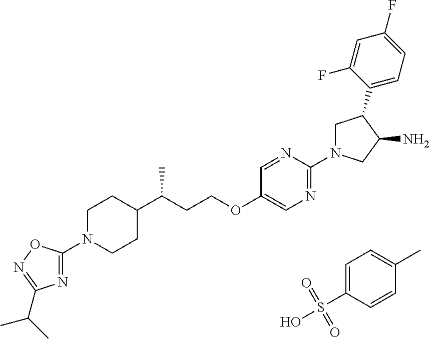

To a solution of [(trans)-4-(2,4-difluorophenyl)-1-(5-{(R)-3-[1-(3-isopropyl-[1,2,4]oxadiazol-5-yl)piperidin-4-yl]butoxy}pyrimidin-2-yl)pyrrolidin-3-yl1carbamic acid tert-butyl ester (Preparation 106, 270 mg, 0.42 mmol) in DCM (10 mL) under argon, cooled to 0° C., was added TFA (2 mL) and the reaction was stirred for 1 h. A further portion of TFA (2 mL) was added and stirring continued at 0° C. for 1 h before quenching the reaction by the addition of a small volume of sat. NaHCO₃ solution. The mixture was dried (Na₂SO₄) and the solvent removed in vacuo. Purification by column chromatography (DCM:MeOH, 100:0, 99:1, 98:2, 97:3, 95:5, 90:10) followed by chiral HPLC (MTBE:EtOH:THF:BA 77:20:3:0.1, 11 mL/min, 250 nm, RT=26.2 min) afforded the title compound as the free base. To a solution of the product in MeOH was added TsOH (1eq.). The mixture was stirred for 5 min before removing the solvent in vacuo to afford the title compound: RT=3.09 min; m/z (ES⁺)=542.4 [M+H]⁺.

### Example 52

### 4-((S)-2-{2-[(3R,4S)-3-Amino-4-(2,5-difluorophenyl)pyrrolidin-1-yl]pyrimidin-5-yloxy}-1-methylethyl)piperidine-1-carboxylic acid isopropyl ester p-toluenesulfonic acid salt

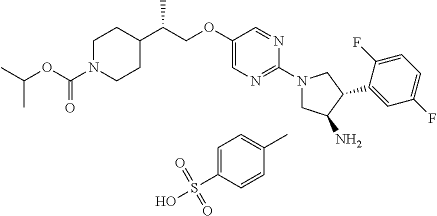

To a solution of 4-[2-(2-chloropyrimidin-5-yloxy)-1-methylethyl]piperidine-1-carboxylic acid isopropyl ester (Preparation 112, 125 mg, 0.35 mmol) and [(3R,4S)-4-(2,5-difluorophenyl)pyrrolidin-3-yl]carbamic acid tert-butyl ester (Preparation 70, 125 mg, 0.42 mmol) in DMSO (0.8 mL), under argon, was added DBU (52 μL, 0.35 mmol) and the reaction was heated to 80° C. for 65 h. The mixture was partitioned between EtOAc (30 mL) and water (30 mL), and the organic phase separated. The aqueous phase was extracted with EtOAc (20 mL) then organic fractions were combined, washed with sat. NaHCO₃ solution (30 mL), brine (30 mL), and dried (MgSO₄), before removal of the solvent in vacuo. Purification by column chromatography (IH:EtOAc, 2:1) followed by chiral HPLC (MTBE:THF 90:10, 13 ml/min, 250 nm) afforded the intermediate product 4-((S)-2-{2-[(3R,4S)-3-tert-butoxycarbonylamino-4-(2,5-difluorophenyl)pyrrolidin-1-yl]pyrimidin-5-yloxy}-1-methylethyl)piperidine-1-carboxylic acid isopropyl ester: RT=4.76 min; m/z (ES⁺)=604.2 [M+H]⁺. To a solution of the product in DCM (5 mL), cooled to 0° C., was added TFA (1 mL) and the reaction stirred at this temperature for 3 h. A further portion of TFA (0.5 mL) was added and stirring continued for 1 h. The crude mixture was passed down an SCX cartridge, eluting with MeOH then NH₄OH in MeOH. The basic fraction was collected and concentrated in vacuo to afford the title compound as the free amine. To a solution of the product in DCM (2 mL) was added a solution of TsOH (1eq.) in MeOH (1 mL), then removal of the solvent in vacuo afforded the title compound: RT=2.94 min; m/z (ES⁺)=504.2 [M+H]⁺

### Example 53

### 4-((R)-2-{2-[(3R,4S)-3-Amino-4-(2,5-difluorophenyl)pyrrolidin-1-yl]pyrimidin-5-yloxy}-1-methylethyl)piperidine-1-carboxylic acid isopropyl ester p-toluenesulfonic acid salt

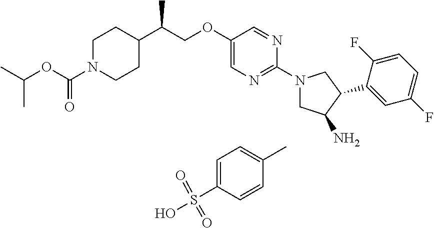

The title compound was prepared from 4-[2-(2-chloropyrimidin-5-yloxy)-1-methylethyl]piperidine-1-carboxylic acid isopropyl ester (Preparation 112) employing the procedure outlined in Example 52. Chiral HPLC: MTBE:THF 90:10, 13 mL/min, 250 nm. LCMS: RT=2.99 min; m/z (ES⁺)=504.2 [M+H]⁺

The biological activity of the compounds of the invention may be tested in the following assay systems:

### GPR119 Yeast Reporter Assay

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Ste3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μL of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di (β-D-galactopyranoside) was used (FDG), a substrate for the enzyme that releases fluorescein, allowing a fluorimetric read-out. 20 μL per well of 500 μM FDG/2.5% Triton X100 was added (the detergent was necessary to render the cells permeable). After incubation of the cells with the substrate for 60 min, 20 μL per well of 1M sodium carbonate was added to terminate the reaction and enhance the fluorescent signal. The plates were then read in a fluorimeter at 485/535 nm.

All of Examples 1 to 52 showed activity in this assay giving an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line was used to investigate the effect of compounds of the invention on intracellular levels of cyclic AMP (cAMP). The cell monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol.

Compounds of the invention produced a concentration-dependent increase in intracellular cAMP level and generally had an EC₅₀ of <10 μM. Compounds showing and EC₅₀ of less than 1 μM in the cAMP assay may be preferred.

### DPP-IV Assay Method

DPP-IV activity was measured by monitoring the cleavage of the fluorogenic peptide substrate, H-Gly-Pro-7-amino-4-methylcoumarin (GP-AMC) whereby the product 7-amino-4-methylcoumarin is quantified by fluorescence at excitation 380 nm and emission 460 nm. Assays were carried out in 96-well plates (Black OptiPlate-96F) in a total volume of 100 μL per well consisting of 50 mM Tris pH 7.6, 100 μM GP-AMC, 10-25 μU recombinant human DPP-IV and a range of inhibitor dilutions in a final concentration of 1% DMSO. Plates were read in a fluorimeter after 30 min incubation at 37° C. Recombinant human DPP-IV residues Asn29-Pro766 was purchased from BioMol.

All of Examples 1 to 53 showed activity in this assay having an IC₅₀ of <20 μM. Compounds of the invention of formula (Ia) generally have an IC₅₀ of <20 μM.

Anti-diabetic effects of compounds of the invention in an in-vitro model of pancreatic beta cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 mL/well and cultured for 24 h and the medium was then discarded. Cells were incubated for 15 min at room temperature with 1000 stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 μL lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μL reactions were set up containing 8 μL sample, 5 μL acceptor bead mix and 12 μL detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells are plated in standard culture medium in 12-well plates at 106 cells/1 ml/well and cultured for 3 days and the medium then discarded. Cells are washed ×2 with supplemented Krebs-Ringer buffer (KRB) containing 119 mM NaCl, 4.74 mM KCl, 2.54 mM CaCl₂, 1.19 mM MgSO₄, 1.19 mM KH₂PO₄, 25 mM NaHCO₃, 10 mM HEPES at pH 7.4 and 0.1% bovine serum albumin. Cells are incubated with lml KRB at 37° C. for 30 min which is then discarded. This is followed by a second incubation with KRB for 30 min, which is collected and used to measure basal insulin secretion levels for each well. Compound dilutions (0, 0.1, 0.3, 1, 3, 10 μM) are then added to duplicate wells in 1 ml KRB, supplemented with 5.6 mM glucose. After 30 min incubation at 37° C. samples are removed for determination of insulin levels. Measurement of insulin was done using the Mercodia Rat insulin ELISA kit, following the manufacturers' instructions, with a standard curve of known insulin concentrations. For each well, insulin levels are corrected by subtraction of the basal secretion level from the pre-incubation in the absence of glucose. Data is analysed using XLfit 3 software.

Compounds of the invention preferably increase insulin secretion at an EC₅₀ of less than 10 μM.

### Oral Glucose Tolerance Tests

The effects of compounds of the invention on oral glucose (Glc) tolerance may be evaluated in male Sprague-Dawley rats. Food is withdrawn 16 h before administration of Glc and remains withdrawn throughout the study. Rats have free access to water during the study. A cut is made to the animals' tails, then blood (1 drop) is removed for measurement of basal Glc levels 60 min before administration of the Glc load. Then, the rats are weighed and dosed orally with test compound or vehicle (20% aqueous hydroxypropyl-β-cyclodextrin) 45 min before the removal of an additional blood sample and treatment with the Glc load (2 g kg⁻¹ p.o.). Blood samples are taken from the cut tip of the tail 5, 15, 30, 60, 120, and 180 min after Glc administration. Blood glucose levels are measured just after collection using a commercially available glucose-meter (OneTouch® Ultra™ from Lifescan). Compounds of the invention preferably statistically reduce the Glc excursion at doses <100 mg kg⁻¹.

The effects of compounds of the invention on oral glucose (Glc) tolerance may also be evaluated in male C57B1/6 or male ob/ob mice. Food is withdrawn 5 h before administration of Glc and remained withdrawn throughout the study. Mice have free access to water during the study. A cut was made to the animals' tails, then blood (20 μL) is removed for measurement of basal Glc levels 45 min before administration of the Glc load. Then, the mice are weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) are then taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood are then added to 180 μL of Trinders glucose reagent (Sigma enzymatic (Trinder) colorimetric method) in a 96-well assay plate. After mixing, the samples are left at room temperature for 30 min before being read against Glc standards (Sigma glucose/urea nitrogen combined standard set). Compounds of the invention preferably statistically reduce the Glc excursion at doses <100 mg kg⁻¹.

1. A compound of formula (I), or a pharmaceutically acceptable salt thereof: 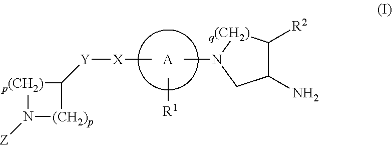
p is 1 or 2;
Z is C(O)OR⁴, C(O)NR⁴R⁵ or heteroaryl which may optionally be substituted by one or two groups selected from the group consisting of C₁₋₄ alkyl, C₃₋₆ cycloalkyl optionally substituted by C₁₋₄ alkyl, C₁₋₄ alkoxy, C₁₋₄ haloalkyl and halogen;
X is selected from the group consisting of CR⁶R⁶⁶, O and NR⁷;
Y is a C₂₋₄ alkylene chain optionally substituted by fluoro or methyl, and when X is CR⁶R⁶⁶ one of the carbons in the alkylene chain may be replaced by O;
A is phenyl or a 6-membered heteroaromatic ring containing one or two nitrogen atoms;
R¹ is hydrogen, halo, cyano, C₁₋₄ alkyl or C₁₋₄ haloalkyl;
q is 1 or 2;
R² is 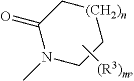
phenyl optionally substituted by one or more halo groups, or pyridyl optionally substituted by one or more halo or methyl groups;
R³ is independently halo or methyl;
n is 0 or 1;
m is 0, 1 or 2;
R⁴ is C₂₋₆ alkyl or C₃₋₆ cycloalkyl optionally substituted by C₁₋₄ alkyl;
R⁵ is hydrogen or C₁₋₄ alkyl;
R⁶ and R⁶⁶ are independently hydrogen, fluoro or C₁₋₄ alkyl; and
R⁷ is hydrogen or C₁₋₄ alkyl.

2. A compound according to claim 1, or a pharmaceutically acceptable salt thereof, having the stereochemistry as defined in formula (Ia):

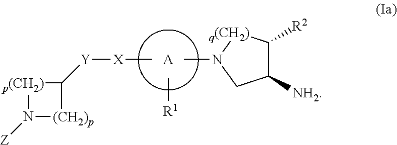

3. A compound according to claim 1, or a pharmaceutically acceptable salt thereof, wherein p is 2.

4. A compound according to claim 1, or a pharmaceutically acceptable salt thereof, wherein z is c(O)OR⁴.

5. A compound according to claim 1, or a pharmaceutically acceptable salt thereof, wherein Z is heteroaryl optionally substituted by one or two groups selected from the group consisting of C₁₋₄ alkyl, C₃₋₆ cycloalkyl optionally substituted by C₁₋₄ alkyl, C₁₋₄ alkoxy, C₁₋₄ haloalkyl and halogen.

6. A compound according to claim 5, or a pharmaceutically acceptable salt thereof, wherein Z is optionally substituted oxadiazole or pyrimidine.

7. A compound according to claim 1, or a pharmaceutically acceptable salt thereof, wherein X is CR⁶R⁶⁶ or O.

8. A compound according to claim 7, or a pharmaceutically acceptable salt thereof, wherein X is O.

9. A compound according to claim 1, or a pharmaceutically acceptable salt thereof, wherein Y is a C₃₋₄ alkylene chain optionally substituted by methyl.

10. A compound according to claim 1, or a pharmaceutically acceptable salt thereof, wherein A is a meta- or para-linked phenyl or a meta- or para-linked 6-membered heteroaromatic ring containing one or two nitrogen atoms.

11. A compound according to claim 10, or a pharmaceutically acceptable salt thereof, wherein A is a para-linked phenyl or a para-linked 6-membered heteroaromatic ring containing one or two nitrogen atoms.

12. A compound according to claim 1, or a pharmaceutically acceptable salt thereof, wherein A is pyridine or pyrimidine.

13. A compound according to claim 1, or a pharmaceutically acceptable salt thereof, wherein R² is phenyl substituted by one or more halo groups.

14. A compound according to claim 13, or a pharmaceutically acceptable salt thereof, wherein R² is phenyl substituted by one or more fluoro groups.

15. (canceled)

16. A pharmaceutical composition comprising a compound according to claim 1, or a pharmaceutically acceptable salt thereof; and a pharmaceutically acceptable carrier.

17. A method for the treatment of a disease or condition in which GPR119 plays a role, said method comprising administering to a subject in need thereof an effective amount of a compound of claim 1, or a pharmaceutically acceptable salt thereof.

18. A method for the treatment of a disease or condition in which GPR119 and DPP-IV play a role, said method comprising administering to a subject in need thereof an effective amount of a compound of claim 1, or a pharmaceutically acceptable salt thereof.

19. A method for the treatment of type II diabetes, said method comprising administering to a subject in need thereof an effective amount of a compound of claim 1, or a pharmaceutically acceptable salt thereof.

20. A method for the treatment of obesity, metabolic syndrome (syndrome X), impaired glucose tolerance, hyperlipidemia, hypertriglyceridemia, hypercholesterolemia, low HDL levels or hypertension, said method comprising administering to a patient in need thereof an effective amount of a compound of claim 1, or a pharmaceutically acceptable salt thereof.

21. A compound according to claim 1, which is:
1-[(3S,4S)-4-Amino-1-(5-{(R)-3-[1-(5-chloropyrimidin-2-yl)piperidin-4-yl]butoxy}pyrimidin-2-yl)pyrrolidin-3-yl]piperidin-2-one or a pharmaceutically acceptable salt thereof,
1-[(3S,4S)-4-Amino-1-(5-{(R)-3-[1-(3-isopropyl-[1,2,4]oxadiazol-5-yl)piperidin-4-yl]butoxy}pyridin-2-yl)pyrrolidin-3-yl]piperidin-2-one p-toluenesulfonic acid salt or a free base thereof,
1-[(3S,4S)-4-Amino-1-(6-{(R)-3-[1-(3-isopropyl-[1,2,4]oxadiazol-5-yl)piperidin-4-yl]butoxy}pyrimidin-4-yl)pyrrolidin-3-yl]piperidin-2-one or a pharmaceutically acceptable salt thereof,
1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]piperidin-2-one p-toluenesulfonic acid salt or a free base thereof,
1-[3S,4S)-4-Amino-1-(5-{(R)-3-[1-(3-isopropyl-[1,2,4]oxadiazol-5-yl)piperidin-4-yl]butoxy}-4-methylpyrimidin-2-yl)pyrrolidin-3-yl]piperidin-2-one p-toluenesulfonic acid salt or a free base thereof,
1-[(3S,4S)-4-Amino-1-(2-{(R)-3-[1-(3-isopropyl-[1,2,4]oxadiazol-5-yl)piperidin-4-yl]butoxy}pyrimidin-5-yl)pyrrolidin-3-yl]piperidin-2-one hydrochloride or a free base thereof,
1-[(3S,4S)-4-Amino-1-(6-{(R)-3-[1-(3-isopropyl-[1,2,4]oxadiazol-5-yl)piperidin-4-yl]butoxy}pyridin-3-yl)pyrrolidin-3-yl]piperidin-2-one hydrochloride or a free base thereof,
1-[(3S,4S)-4-Amino-1-(5{(R)-3-[1-(3-isopropyl-[1,2,4]oxadiazol-5-yl)piperidin-4-yl]butoxy}pyrimidin-2-yl)pyrrolidin-3-yl]-4-methylpiperidin-2-one hydrochloride or a free base thereof,
1-[(3S,4S)-4-Amino-1-(6-{(R)-3-[1-(3-isopropyl-[1,2,4]oxadiazol-5-yl)piperidin-4-yl]butoxy}pyridazin-3-yl)pyrrolidin-3-yl]piperidin-2-one hydrochloride or a free base thereof,
1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]-5-methylpiperidin-2-one or a pharmaceutically acceptable salt thereof,
(R)-1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]-5-methylpiperidin-2-one or a pharmaceutically acceptable salt thereof,
(S)-1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]-5-methylpiperidin-2-one or a pharmaceutically acceptable salt thereof,
1-[3S,4S)-4-Amino-1-(5-{(R)-3-[1-(3-isopropyl-[1,2,4]oxadiazol-5-yl)piperidin-4-yl]butoxy}pyrazin-2-yl)pyrrolidin-3-yl]piperidin-2-one or a pharmaceutically acceptable salt thereof,
(3R,4S)-4-(2,5-Difluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine p-toluenesulfonic acid salt or a free base thereof,
4-((R)-3-{2-[(3S,4S)-3-Amino-4-(2-oxopiperidin-1-yl)pyrrolidin-1-yl]pyrimidin-5-yloxy}-1-methylpropyl)piperidine-1-carboxylic acid isopropyl ester p-toluenesulfonic acid salt or a free base thereof,
1-[(3S,4S)-4-Amino-1-(5-{(R)-3-[1-(3-isopropyl-[1,2,4]oxadiazol-5-yl)piperidin-4-yl]butoxy}-3-methylpyridin-2-yl)pyrrolidin-3-yl]piperidin-2-one p-toluenesulfonic acid salt or a free base thereof,
4-((R)-3-{2-[(3R,4S)-3-Amino-4-(2,5-difluorophenyl)pyrrolidin-1-yl]pyrimidin-5-yloxy}-1-methylpropyl)piperidine-1-carboxylic acid isopropyl ester p-toluenesulfonic acid salt or a free base thereof,
4-((R)-3-{2-[(3R,4S)-3-Amino-4-(2,4-difluorophenyl)pyrrolidin-1-yl]pyrimidin-5-yloxy}-1-methylpropyl)piperidine-1-carboxylic acid isopropyl ester p-toluenesulfonic acid salt or a free base thereof,
4-((R)-3-{2-[(3S,4R)-3-Amino-4-(2,4-difluorophenyl)pyrrolidin-1-yl]pyrimidin-5-yloxy}-1-methylpropyl)piperidine-1-carboxylic acid isopropyl ester p-toluenesulfonic acid salt or a free base thereof,
[(3R,4S)-1-(5-{(R)-3-[1-(5-Chloropyrimidin-2-yl)piperidin-4-yl]butoxy}pyrimidin-2-yl)-4-(2,5-difluorophenyl)pyrrolidin-3-ylamine p-toluenesulfonic acid salt or a free base thereof,
(3R,4S)-4-(2,5-Difluorophenyl)-1-(6-{(R)-3-[1-(3-isopropyl-[1,2,4]oxadiazol-5-yl)piperidin-4-yl]butoxy}pyrimidin-4-yl)pyrrolidin-3-ylamine or a pharmaceutically acceptable salt thereof,
1-[(3S,4S)-4-Amino-1-(5{(R)-3-[1-(3-isopropyl-[1,2,4]oxadiazol-5-yl)piperidin-4-yl]butoxy}pyrimidin-2-yl)pyrrolidin-3-yl]-(S)-4-methylpiperidin-2-one or a pharmaceutically acceptable salt thereof,
1-[(3S,4S)-4-Amino-1-(5{(R)-3-[1-(3-isopropyl-[1,2,4]oxadiazol-5-yl)piperidin-4-yl]butoxy}pyrimidin-2-yl)pyrrolidin-3-yl]-(R)-4-methylpiperidin-2-one p-toluenesulfonic acid salt or a free base thereof,
(3′S,4′S)-4′-Amino-1′-(5-{(R)-3-[1-(3-isopropyl-[1,2,4]oxadiazol-5-yl)piperidin-4-yl]butoxy}pyrimidin-2-yl)-[1,3′]bipyrrolidinyl-2-one p-toluenesulfonic acid salt or a free base thereof,
1-[(3S,4S)-4-Amino-1-(5-{(R)-3-[1-(3-isopropyl-[1,2,4]oxadiazol-5-yl)piperidin-4-yl]butoxy}pyrimidin-2-yl)pyrrolidin-3-yl]-5,5-difluoropiperidin-2-one p-toluenesulfonic acid salt or a free base thereof,
4-((R)-3-{2-[(3R,4R)-3-Amino-4-(2,5-difluorophenyl)piperidin-1-yl]pyrimidin-5-yloxy}-1-methylpropyl)-piperidine-1-carbocylic acid isopropyl ester p-toluenesulfonic acid salt or a free base thereof,
(3R,4R)-4-(2,5-Difluorophenyl)-1-(5-{(R)-3-[1-(3-isopropyl-[1,2,4]oxadiazol-5-yl)piperidin-4-yl]butoxy}pyrimidin-2-yl)piperidin-3-ylamine p-toluenesulfonic acid salt or a free base thereof,
1-[(3S,4S)-4-Amino-1-(5-{3-[1-(3-isopropyl-[1,2,4]oxadiazol-5-yl)piperidin-4-yl]propoxy}pyrimidin-2-yl)pyrrolidin-3-yl]piperidin-2-one or a pharmaceutically acceptable salt thereof,
(3R,4S)-4-(2-Fluorophenyl)-1-(5-{3-[1-(3-isopropyl-[1,2,4]oxadiazol-5-yl)piperidin-4-yl]propoxy}pyrimidin-2-yl)pyrrolidin-3-ylamine p-toluenesulfonic acid salt or a free base thereof,
(3R,4S)-4-(2-Fluorophenyl)-1-(5-{(R)-3-[1-(3-isopropyl-[1,2,4]oxadiazol-5-yl)piperidin-4-yl]butoxy}-4-methylpyrimidin-2-yl)pyrrolidin-3-ylamine p-toluenesulfonic acid salt or a free base thereof,
4-((R)-3-{2-[(3R,4S)-3-Amino-4-(2-fluorophenyl)pyrrolidin-1-yl]pyrimidin-5-yloxy}-1-methylpropyl)piperidine-1-carboxylic acid isopropyl ester p-toluenesulfonic acid salt or a free base thereof,
(3R,4S)-1-(5-{(R)-3-[1-(3-Ethyl-[1,2,4]oxadiazol-5-yl)piperidin-4-yl]butoxy}pyrimidin-2-yl)-4-(2-fluorophenyl)pyrrolidin-3-ylamine p-toluenesulfonic acid salt or a free base thereof,
(3R,4S)-1-(5-{(R)-3-[1-(3-Isopropyl-[1,2,4]oxadiazol-5-yl)piperidin-4-yl]butoxy}pyrimidin-2-yl)-4-(2,4,5-trifluorophenyl)pyrrolidin-3-ylamine p-toluenesulfonic acid salt or a free base thereof,
(3R,4S)-1-(5-{(R)-3-[1-(3-Ethyl-[1,2,4]oxadiazol-5-yl)piperidin-4-yl]butoxy}pyrimidin-2-yl)-4-(2,4,5-trifluorophenyl)pyrrolidin-3-ylamine p-toluenesulfonic acid salt or a free base thereof,
(3R,4S)-4-(2,5-Difluorophenyl)-1-(5-{(R)-3-[1-(3-isopropyl-[1,2,4]oxadiazol-5-yl)piperidin-4-yl]butoxy}-3-methylpyridin-2-yl)pyrrolidin-3-ylamine p-toluenesulfonic acid salt or a free base thereof,
(3R,4S)-1-(5-{(R)-3-[1-(5-Chloropyrimidin-2-yl)piperidin-4-yl]butoxy}pyrimidin-2-yl)-4-(2-fluorophenyl)pyrrolidin-3-ylamine p-toluenesulfonic acid salt or a free base thereof,
(3R,4R)-4-(2,5-Difluorophenyl)-5′-{(R)-3-[1-(3-isopropyl-[1,2,4]oxadiazol-5-yl)piperidin-4-yl]butoxy}-3,4,5,6-tetrahydro-2H-[1,2′]bipyridinyl-3-ylamine dihydrochloride or a free base thereof,
(3R,4R)-4-(2,5-Difluorophenyl)-1-(5-{(R)-3-[1-(3-isopropyl-[1,2,4]oxadiazol-5-yl)piperidin-4-yl]butoxy}pyrazin-2-yl)piperidin-3-ylamine hydrochloride or a free base thereof,
(3R,4R)-4-(2,5-Difluorophenyl)-5′-{3-[1-(3-isopropyl-[1,2,4]oxadiazol-5-yl)piperidin-4-yl]propoxy}-3,4,5,6-tetrahydro-2H-[1,2′]bipyridinyl-3-ylamine dihydrochloride or a free base thereof,
(3R,4R)-4-(2,5-Difluorophenyl)-1-(5-{3-[1-(3-isopropyl-[1,2,4]oxadiazol-5-yl)piperidin-4-yl]propoxy}pyrimidin-2-yl)piperidin-3-ylamine p-toluenesulfonic acid salt or a free base thereof,
(3R,4R)-1-(5-{3-[1-(5-Chloropyrimidin-2-yl)piperidin-4-yl]propoxy}pyrimidin-2-yl)-4-(2,5-difluorophenyl)piperidin-3-ylamine p-toluenesulfonic acid salt or a free base thereof,
(3R,4R)-1-(5-{3-[1-(5-Chloropyrimidin-2-yl)piperidin-4-yl]propoxy}pyrimidin-2-yl)-4-(2-fluorophenyl)piperidin-3-ylamine or a pharmaceutically acceptable salt thereof,
4-((R)-3-{2-[(3R,4R)-3-Amino-4-(2-fluorophenyl)piperidin-1-yl]pyrimidin-5-yloxy}-1-methylpropyl)piperidine-1-carboxylic acid isopropyl ester or a pharmaceutically acceptable salt thereof,
(3R,4S)-1-(5-{3-[1-(5-isopropyl-[1,2,4]oxadiazol-3-yl)piperidin-4-yl]propoxy}pyrimidin-2-yl)-4-(2,4,5-trifluorophenyl)pyrrolidin-3-ylamine or a pharmaceutically acceptable salt thereof,
4-((R)-3-{2-[(3R,4S)-3-Amino-4-(2,4,5-trifluorophenyl)pyrrolidin-1-yl]pyrimidin-5-yloxy}-1-methylpropyl)piperidine-1-carboxylic acid isopropyl ester hydrochloride or a free base thereof,
(3R,4R)-4-(2-Fluorophenyl)-1-(5-{(R)-3-[1-(3-isopropyl-[1,2,4]oxadiazol-5-yl)piperidin-4-yl]butoxy}pyrimidin-2-yl)piperidin-3-ylamine hydrochloride or a free base thereof,
(3R,4S)-1-(5-{3-[1-(3-Isopropyl-[1,2,4]oxadiazol-5-yl)piperidin-4-yl]propoxy}pyrimidin-2-yl)-4-(2,4,5-trifluorophenyl)pyrrolidin-3-ylamine or a pharmaceutically acceptable salt thereof,
(3R,4S)-4-(2-Fluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine or a pharmaceutically acceptable salt thereof,
(3S,4R)-4-(2-Fluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine or a pharmaceutically acceptable salt thereof,
4-((R)-4-{2-[(3S,4S)-3-Amino-4-(2-oxopiperidin-1-yl)pyrrolidin-1-yl]pyrimidin-5-yl}-1-methylbutyl)piperidine-1-carboxylic acid isopropyl ester or a pharmaceutically acceptable salt thereof,
(3R,4S)-4-(2,4-Difluorophenyl)-1-(5-{(R)-3-[1-(3-isopropyl-[1,2,4]oxadiazol-5-yl)piperidin-4-yl]butoxy}pyrimidin-2-yl)pyrrolidin-3-ylamine p-toluenesulfonic acid salt or a free base thereof,
4-((S)-2-{2-[(3R,4S)-3-Amino-4-(2,5-difluorophenyl)pyrrolidin-1-yl]pyrimidin-5-yloxy}-1-methylethyl)piperidine-1-carboxylic acid isopropyl ester p-toluenesulfonic acid salt or a free base thereof, or
4-((R)-2-{2-[(3R,4S)-3-Amino-4-(2,5-difluorophenyl)pyrrolidin-1-yl]pyrimidin-5-yloxy}-1-methylethyl)piperidine-1-carboxylic acid isopropyl ester p-toluenesulfonic acid salt or a free base thereof.
